(19) United States
(12) Patent Application Publication
(10) Pub. No.: US 2014/0357497 A1
(43) Pub. Date: Dec. 4, 2014

(54) DESIGNING PADLOCK PROBES FOR TARGETED GENOMIC SEQUENCING

(71) Applicants: Kun Zhang, San Diego, CA (US); Athurva Gore, San Diego, CA (US)

(72) Inventors: Kun Zhang, San Diego, CA (US); Athurva Gore, San Diego, CA (US)

(21) Appl. No.: 14/113,449

(22) PCT Filed: Apr. 26, 2012
(86) PCT No.: PCT/US12/35227

§ 371 Date: Aug. 21, 2014

Related U.S. Application Data
(60) Provisional application No. 61/479,747, filed on Apr. 27, 2011.

(51) Int. Cl. C12Q 1/68

(52) U.S. Cl. CPC C12Q 1/6811
USPC 506/1; 506/9; 506/37

(57) ABSTRACT

Methods, systems, and computer programs for designing probes or primers for nucleic acid sequencing, generating libraries of nucleic acid sequences, and mapping genomic sequences are provided herein,

### SEQUENCE LISTING

The instant application contains a Sequence Listing which has been submitted in ASCII format via EFS-Web and is hereby incorporated by reference in its entirety. Said ASCII copy, created on Apr. 26, 2012, is named 378665WO-1.txt and is 99,190 bytes in size.

### TECHNICAL FIELD

The subject matter described herein relates generally to the fields of molecular biology and bioinformatics. More specifically, the subject matter described herein relates to systems, methods, and computer programs for designing probes for use in nucleic acid sequencing, particularly padlock probes useful in carrying out targeted genomic and methylation sequencing.

### BACKGROUND

Padlock Probe (PP) technology is a multiplex genomic enrichment method allowing for accurate targeted high-throughput sequencing. PP technology has been used to perform highly multiplexed genotyping, digital allele quantification, targeted bisulfate sequencing, and exome sequencing. See Hardenbol, P. et al., (2005) Genome Res. 15: 269-275; Wang, Y. et al., (2005) Nucleic Acids Res., 2005, 33(21) e183: 1-14; Porreca, G. J. et al., (2007) Nat. Methods 4(11): 931-936; Zhang, K. et al., (2009) Nat. Methods 6: 613-618; Turner, E. H. et al., (2009) Nat. Methods 6: 315-316; Deng, J. et al., (2009) Nat. Biotechnol. 27: 353-360; Shoemaker, R. et al., (2010) Genome Res. 20: 883-889, and Gore, A. et al., (2011) Nature 471(7336): 63-67.

Padlock probe technology may utilize a linear oligonucleotide molecule with two binding sequences at each end joined by a common linker sequence. The probe's binding arms may be hybridized several base pairs apart surrounding a target single-stranded genomic DNA region. A DNA polymerase (with each of the four standard dNTP molecules) may be used to fill in the gap between the two binding arms, and a DNA ligase may be used to circularize the resulting molecule. A mixture of exonucleases may then used to digest all arm; the resulting circular molecules can be amplified using rolling circle amplification or polymerase chain reaction to generate a DNA library compatible with modern high-throughput sequencers.

Previous padlock probe work has relied on relatively arbitrary methods for padlock probe design. Padlock probes are generally designed to have binding arms with complementary sequence around a chosen target region of approximately 100-200 base pairs; each binding arm is generally designed to have a specific DNA melting temperature. However, padlock probe efficacy from molecule to molecule has been found to vary greatly, ranging across several orders of magnitude. Previous work identified a complex nonlinear relationship between padlock probe efficiency and many probe characteristics. (Deng, J. et al., (2009) Nat. Biotechnol. 27: 353-360; Li, J. B. et al., (2009) Genome Res. 19(9): 1606-1615). The bias inherent in padlock probe design has been demonstrated to show a complex nonlinear relationship with many probe characteristics, confounding attempts to generate efficient probes. Because of this, padlock probe results are highly biased towards certain genomic regions. Certain genomic regions are therefore extremely difficult to target using padlock probes due to the lack of knowledge of probe capturing efficiency.

There remains a need in the art for methods and algorithms that are useful for designing probes from a large set of probe characteristics to ensure optimal capture of DNA sequences, particularly genomic DNA.

### SUMMARY

The subject matter described herein relates to methods, systems, and computer program products that can be used to design oligonucleotide primers and probes, particularly padlock probes for high-density multiplex genome sequencing. The subject matter disclosed herein is particularly useful for detecting methylation status and/or single nucleotide variants in a target nucleic acid sequence of interest.

Accordingly, in some aspects, the subject matter described herein provides a method of designing probes or primers for sequencing a target nucleic acid molecule, comprising the steps of selecting one or more inputs associated with efficiency of the probe or primer; selecting a target nucleic acid sequence; generating a first library of probe or primer sequences that comprise a target capturing sequence that is complementary to the target nucleic acid sequence; determining the efficiency of each probe or primer sequence in the first library by using an algorithm comprising the one or more selected inputs defined herein; ranking the probe or primer sequences in the first library by efficiency; extracting the probe or primer sequences having the highest efficiency to generate a second library; and adding a linker sequence to each of the probe or primer sequences in the second library. In some embodiments, the method further comprises synthesizing the probe or primer.

In some embodiments, the probe is a padlock probe. The one or more inputs may comprise target length, target folding energy, target GC content, extension arm A %, extension arm G %, target A %, target T %, target G %, number of “GG” dinucleotides in ligation arm, number of “AT” dinucleotides in extension arm, number of “GG” dinucleotides in extension arm, number of “AA” dinucleotides in target, number of “AT” dinucleotides in target, number of “TA” dinucleotides in target, number of “GT” dinucleotides in target, number of “GA” dinucleotides in target, ligation arm terminal dinucleotide, extension arm terminal dinucleotide, target 5′ terminal dinucleotide, ligation arm melting temperature, extension arm melting temperature, ligation arm length, extension arm length, local single-stranded folding energy of the target, and dinucleotides present at the extension site and ligation site during probe capture.

In some embodiments, the target nucleic acid sequence is derived from a human.

The target-capturing sequence may include a ligation arm and an extension arm. In some embodiments, the target-capturing sequence contains one or more CpG dinucleotides. The target-capturing sequences in the first library may also contain all possible methylation state combinations of the one or more CpG dinucleotides. In some embodiments, the extension arm comprises one or more priming sites for amplification of the target nucleic acid sequence and may be universal priming sites. The target capturing sequence may also include one or more restriction sites.

The methods disclosed herein involve an algorithm that may comprise one or more neural networks. The one or more neural networks may comprise the one or more inputs, e.g., seven or more inputs.

In certain embodiments, the method further comprises, after the extracting step, pooling the non-extracted probe or primer sequences and repeating certain steps defined herein.

In some embodiments, the linker sequence is a sequence that is common to each probe or primer sequence in the second library.

In another aspect, an apparatus is provided, comprising at least one processor and at least one memory including code which when executed by the at least one processor provides operations comprising: selecting one or more inputs associated with efficiency of the probe or primer; selecting a target nucleic acid sequence; generating a first library of probe or primer sequences that comprise a target capturing sequence that is complementary to the target nucleic acid sequence; determining the efficiency of each probe or primer sequence in the first library by using an algorithm comprising the one or more selected inputs; ranking the probe or primer sequences in the first library by efficiency; extracting the probe or primer sequences having the highest efficiency to generate a second library; and adding a linker sequence to each of the probe or primer sequences in the second library.

In another aspect of the subject matter described herein, a computer-readable storage medium including code is provided, which when executed by at least one processor provides operations comprising: selecting one or more inputs associated with efficiency of the probe or primer; selecting a target nucleic acid sequence; generating a first library of probe or primer sequences that comprise a target capturing sequence that is complementary to the target nucleic acid sequence; determining the efficiency of each probe or primer sequence in the first library by using an algorithm comprising the one or more selected inputs; ranking the probe or primer sequences in the first library by efficiency; extracting the probe or primer sequences having the highest efficiency to generate a second library; and adding a linker sequence to each of the probe or primer sequences in the second library.

Other features and advantages of the subject matter described herein will be apparent from the following detailed description and claims.

### BRIEF DESCRIPTION OF THE DRAWINGS

The following Detailed Description, given by way of example, but not intended to limit the subject matter described herein to specific embodiments described, may be understood in conjunction with the accompanying figures, incorporated herein by reference, in which:

FIG. 1 is a block diagram of a single padlock probe capture experiment, demonstrating an example of a workflow for targeted genomic resequencing consistent with some of the exemplary embodiments described herein;

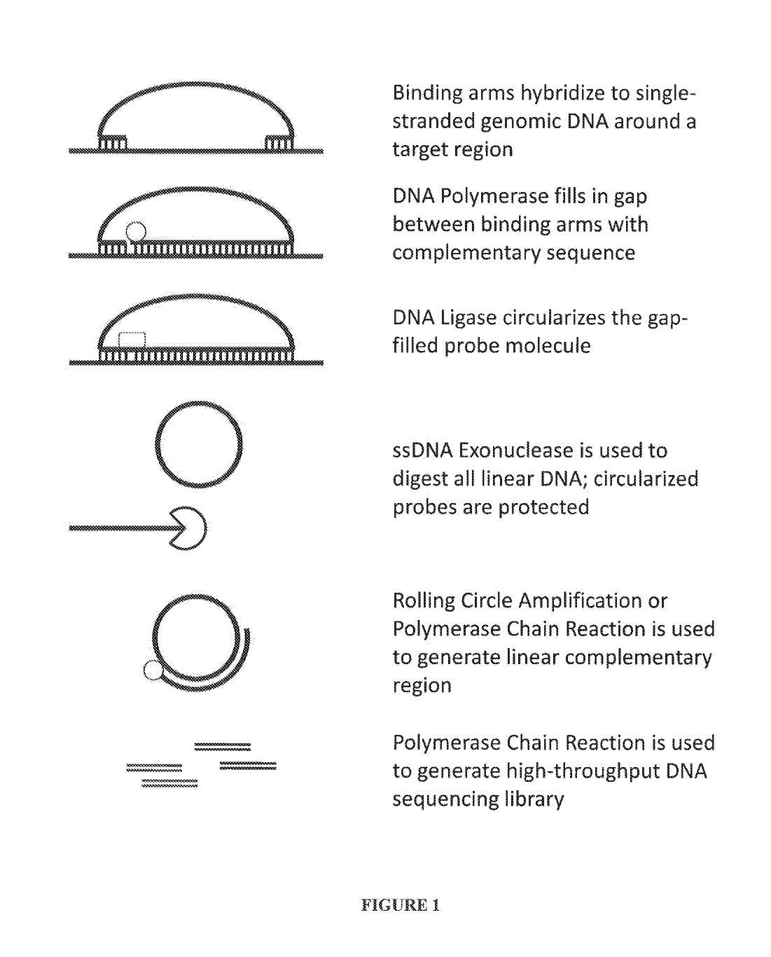

FIG. 2 is a flowchart of a process for designing padlock probes from input files consistent with some of the exemplary embodiments described herein;

FIG. 3 is a block diagram of a back propagation neural network used to derive the probe efficiency scoring equation consistent with some of the exemplary embodiments described herein;

FIG. 4 is a depiction of two examples of customized linker sequences and the function provided thereby, each of which is consistent with some of the exemplary embodiments described herein. FIG. 4 discloses SEQ ID NOS 420-424, respectively, in order of appearance;

FIGS. 5A-5E depicts the design of padlock probes for targeted bisulfite sequencing. (A) is a diagram showing that each padlock probe has a common linker sequence flanked by two target-specific capturing arms (H1 and H2). H1 and H2 are melting temperature normalized, and a spacer sequence is included to normalize probe lengths. The linker sequence contains priming sites (AP1 and AP2) for universal primers, two MmeI sites and a central AluI recognition site. (B) is a diagram depicting a CpG island (or other target region) covered by multiple padlock probes targeting partially overlapped regions on alternating strands. (C) is a diagram showing a library of padlock probes annealed to bisulfite-converted genomic DNA. (D) is a flow chart showing the generation of a shotgun sequencing library. (E) is a picture showing gel electrophoresis analysis of the padlock-captured products from two independent capturing reactions (1 and 2) and a no-template control (NTC).

FIGS. 6A-6G are graphs summarizing an analysis of the effect of probe characteristics on capturing efficiency. The statistical package R and its effects module were used for this analysis. A linear model was used, and each individual factor was assumed to be independent. The dashed lines represent a 95% confidence interval. (A) High GC content in the ligation arm was found to increase probe capturing efficiency. (B) Longer ligation arms were found to capture probes with higher efficiency than short ligation arms. (C) The frequency of 12-mers within the ligation arm in the human genome did not have a statistically significant effect on probe efficiency. (D) GC content of the extension arm did not have a statistically significant effect on probe efficiency. (E) Extension arm length did not have a statistically significant effect on probe efficiency; the trend was that a longer extension arm would be better. (F) The frequency of 12-mers within the extension arm in the human genome did not have a statistically significant effect on probe efficiency. (G) Shorter target sequences were captured with higher efficiency than long target sequences.

FIGS. 7A-7D depict experimental normalization of padlock-capturing efficiency. (A) shows the “subsetting” strategy. (B) depicts the ‘suppressor oligo’ strategy. (C) shows the distribution of normalized abundance for all captured targets with one 30,000-probe set and with four probe sets. The x-axis is the normalized abundance of each captured target, which is calculated by dividing the counts of the target by the average counts of all targets. The y-axis is the fraction of probes with the coverage equal to or greater than the normalized coverage. (D) Comparison of relative abundance for each target before and after normalization. The vertical dash lines indicate the clear separation of four subsets of targets, as well as the fifth set normalized with the suppressor oligos.

FIGS. 8A-8C show the results of a validation experiment demonstrating the digital methylation assay. (A) Comparison of methylation measurements from both strands for the same CpG sites. The methylation levels of the forward strand were plotted against the levels of the reverse strand on 2697 CpG sites that were covered by on both strands by different probes. (B) Methylation levels of 182 randomly selected CpG sites in the BJ fibroblast lines were measured by the conventional bisulfite Sanger sequencing. (C) Comparison of the methylation levels of 25,665 CpG sites (at least 50× sequencing reads per site) between two biological replicates on the IMR90 fibroblasts.

FIG. 9 is a schematic for the probe design software (ppDesigner).

FIG. 10 are graphs comparing probe capture efficiencies between the DMR220K, LC4K probe sets and the CGI30K set. The first three plots were generated from data without subsetting or suppressor oligos to allow for a direct comparison of probe design.

FIG. 11 is a scatter plot of number of characterized CpG sites versus mappable sequencing data for the DMR330K probe set. Variability in sequencing quality of individual sequencing runs is responsible for the different number of CpG sites characterized with similar sequencing effort.

FIG. 12 is a graph showing the number of CpG sites called per sample as a function of sequencing effort. The horizontal dash line represents 4 Gbps of sequences per library.

FIGS. 13A-13B are graphs showing captured CpG sites that were tested for potential regulatory interactions with genes by GREAT. (A) Most CpG sites were interacting with 1-2 genes. (B) Distance of CpG sites to the transcriptional start sites (TSS) of the predicted regulating genes.

FIGS. 14A-14E are graphs showing the accuracy of digital quantification by BSPP. (A, B) show a comparison of the methylation levels obtained at 10× depth from multiple capture reactions of the same sample (PGPliPS) within batches and between batches. (C, D, E) Within sample comparison of methylation levels obtained from different probes capturing the same CpG site on different strands at 10× depth within one capture reaction.

FIG. 15 is a graph showing the comparison between BSPP and whole genome bisulfite sequencing (WGBS). Two H1 ESC datasets were compared, using sites with at least 10× read depth in each.

FIG. 16 is a graph depicting the variation in amount of sequencing data obtained per sample in a multiplexed BSPP capture experiment. Forty-eight whole blood samples were captured and sequenced in one batch using the library-free BSPP method.

FIG. 17 depicts exemplary padlock probes ordered from (A) Agilent's oligonucleotide synthesis service (SEQ ID NOS 425-427, respectively, in order of appearance) and (B) LC Sciences' oligonucleotide synthesis service (SEQ ID NOS 428-430, respectively, in order of appearance).

FIG. 18 is a diagram depicting the addition of a second neural network specifically for bisulfite-converted DNA. This network contains two hidden layers with 10 and 12 nodes, respectively, and accepts 25 pieces of information as input.

### DETAILED DESCRIPTION

The features, structures, or characteristics described throughout this specification may be combined in any suitable manner in one or more embodiments. For example, the usage of the phrases “exemplary embodiments,” “example embodiments,” “some embodiments,” or other similar language, throughout this specification refers to the fact that a particular feature, structure, or characteristic described in connection with an embodiment may be included in at least one embodiment described herein. Thus, appearances of the phrases “exemplary embodiments,” “example embodiments,” “in some embodiments,” “in other embodiments,” or other similar language, throughout this specification do not necessarily all refer to the same group of embodiments, and the described features, structures, or characteristics can be combined in any suitable manner in one or more embodiments:

To facilitate the understanding of this disclosure, a number of terms are defined below. Terms defined herein have meanings as commonly understood by a person of ordinary skill in the areas relevant to the subject matter described herein. Terms such as “a”, “an” and “the” are not intended to refer to only a singular entity, but include the general class of which a specific example may be used for illustration. The terminology herein is used to describe specific embodiments of the subject matter described herein, but their usage does not delimit the subject matter, except as outlined in the claims.

The term “read” refers to a nucleic acid sequence of sufficient length (e.g., at least about 30 bp) that can be used to identify a larger sequence or region, e.g. that can be aligned and specifically assigned to a chromosome or genomic region or gene.

As used herein, the terms “aligned”, “alignment”, or “aligning” refer to one or more sequences that are identified as a match in terms of the order of their nucleic acid molecules to a known sequence from a reference genome. Such alignment can be done manually or by a computer algorithm. Examples include, without limitation, the Efficient Local Alignment of Nucleotide Data (ELAND) computer program distributed as part of the Illumina Genomics Analysis, Bowtie, BWA, and SOAP2Align. The matching of a sequence read in aligning can be a 100% sequence match or less than 100% (non-perfect match).

“Amplification” or “amplifying” methods include but are not limited to the polymerase chain reaction (PCR), the ligase chain reaction (LCR) (e.g., Wu, D. Y. and Wallace, R. B. (1989) Genomics 4: 560-569; Landegren, U. et al., (1998) Science 241(4869): 1077-1080; and Barringer, K. J. et al. (1990) Gene 89(1): 117-122), transcription amplification (Kwoh, D. Y. et al., (1989) Proc. Natl. Acad. Sci. 86(4): 1173-1177 and WO88/10315), self-sustained sequence replication (Guatelli, J. C. et al., (1990) Proc. Nat. Acad. Sci. 87(5): 1874-1878 and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA; U.S. Pat. Nos. 5,409,818, 5,554,517, and 6,063,603 each of which is incorporated herein by reference). Other amplification methods that may be used are described in U.S. Pat. Nos. 5,242,794, 5,494,810, 4,988,617. Additional methods of sample preparation and techniques for reducing the complexity of a nucleic sample are described in Dong, S. et al., (2001) Genome Res. 11(8): 1418-1424, in U.S. Pat. Nos. 6,361,947, 6,391,592 and U.S. patent application Ser. Nos. 09/916,135, 09/920,491, 09/910,292, and Ser. No. 10/013,598.

A “nucleotide” is a monomer that includes a base, such as a pyrimidine, purine, or synthetic analogs thereof, linked to a sugar and one or more phosphate groups. Nucleotides include adenine (A) residues, guanine (G) residues, cytosine (C) residues, thymine (T) residues, and uracil (U) residues. The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U). Nucleotides also include chemical entities containing modified bases, modified sugar moieties and modified phosphate backbones, for example as described in U.S. Pat. No. 5,866,336. Such modifications however, can allow for incorporation of the nucleotide into a growing nucleic acid chain or for binding of the nucleotide to the complementary nucleic acid chain.

Nucleotides can be modified at any position on their structures. Examples include, but are not limited to, the modified nucleotides 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N-6-sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyarninomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, and 2,6-diaminopurine.

Examples of modified sugar moieties which can be used to modify nucleotides at any position on their structures include, but are not limited to: arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

As used herein the terms “nucleic acid”, “nucleic acid molecule”, “polynucleotide” and “oligonucleotide” are used interchangeably and refers to the polymeric form of nucleotides, either ribonucleotides and/or deoxyribonucleotides or a modified form of either type of nucleotide. Nucleic acids include, without limitation, cDNA, mRNA, genomic DNA, and synthetic (such as chemically synthesized) DNA or RNA, plasmids, amplicons, cosmids, and fragments thereof. The nucleic acid can be double stranded (ds) or single stranded (ss). Where single stranded, the nucleic acid molecule can be the sense strand or the antisense strand. Nucleic acids can include natural nucleotides (such as adenine, thymine/uracil, cytosine, and guanine) and can also include analogs of natural nucleotides. A set of bases linked to a peptide backbone, as in a peptide nucleic acid (PNA), can be used as a substitute for a nucleic acid molecule. Nucleic acids can be modified by means of a fluorophore that is directly or indirectly excitable. “Fluorescent DNA dye” as used herein may refer to a composition, for example SYBR Green I or SYBR Gold that becomes fluorescently excitable when it associates with double-stranded DNA. Other examples of fluorescent DNA dyes are known in the art and include, e.g., 5-carboxyfluorescein, 2′7′-dimethoxy-4′5′-dichloro-6-carboxyfluorescein, fluorescein (FL); N,N,N′,N′-tetramethyl-6-carboxyrhodamine; 6-carboxy-X-rhodamine; CY3; CY5; tetrachloro-fluorescein; and hexachloro-fluorescein; NED; 6-FAM; VIC; PET; LIZ, SID, TED, and TAZ.

A “target” nucleic acid molecule is a nucleic acid to be sequenced, identified, or detected, and can be obtained or isolated in purified form, by any method known to those skilled in the art (for example, as described in U.S. Pat. No. 5,674,743), but need not be in purified form. Various other biomolecules can also be present with the target nucleic acid molecule. For example, the target nucleic acid molecule can be present in a cell or a biological sample (which can include other nucleic acid molecules and proteins). The target nucleic acid molecule may be a whole genome or a portion of a genome, such as chromosomal sequences, or may be extrachromosomal sequences such as plasmids. The target nucleic acid molecule can be derived from any species, including but not limited to, vertebrates such as humans, cows, dogs, cats, mice, rats, sheep, horse, goat, invertebrates, bacteria, viruses, fungi, and the like. The target nucleic acid may be derived from any source, including tissues, primary cells, cultured cells, cell lines, tumor specimens, bodily fluids, and the like. The target nucleic acid may also be wholly or partly synthetic. A “complementary” nucleic acid molecule is complementary to the target nucleic acid molecule and is the nucleic acid strand that is elongated when sequencing the target nucleic acid molecule.

An “oligonucleotide” refers to a linear nucleic acid molecule (such as DNA or RNA) sequence of at least 6 nucleotides, for example at least 9, at least 15, at least 18, at least 24, at least 30, at least 50, at least 100, at least 200 or even at least 500 nucleotides long. However shorter or longer oligonucleotides may be used. Oligonucleotides may be designed to have different length. In some embodiments, the sequence of the nucleic acid molecule may be divided up into a plurality of shorter sequences that can be synthesized in parallel and assembled into a single or a plurality of desired nucleic acid molecules using the methods described herein. In certain embodiments, the oligonucleotides are designed to provide the full sense and antisense strands of the nucleic acid molecule. After hybridization of the plus and minus strand oligonucleotides, two double stranded oligonucleotides are subjected to ligation or polymerization in order to form a first subassembly product. Subassembly products are then subjected to ligation or polymerization to form a larger DNA or the full DNA sequence.

A “primer” is a short nucleic acid molecule, for example sequences of at least 9 nucleotides, which can be annealed to a complementary target nucleic acid molecule by nucleic acid hybridization to form a hybrid between the primer and the target nucleic acid strand. A primer can be extended along the target nucleic acid molecule by a polymerase enzyme. Therefore, individual primers can be used for nucleic acid sequencing, wherein the sequence of the primer is specific for the target nucleic acid molecule, for example so that the primer will hybridize to the target nucleic acid molecule under stringent hybridization conditions. In particular examples, a primer is at least 10 nucleotides in length, such as at least 10 contiguous nucleotides complementary to a target nucleic acid molecule to be sequenced. In order to enhance specificity, longer primers can be employed, such as primers having at least 12, at least 15, at least 20, or at least 30 contiguous nucleotides complementary to a target nucleic acid molecule to be sequenced. Methods for preparing and using primers are described in, for example, Sambrook et al. (1989) Molecular Cloning: A Laboratory Manual, Cold Spring Harbor, N.Y.; Ausubel et al. (1987) Current Protocols in Molecular Biology, Greene Publ. Assoc. & Wiley-Intersciences.

As used herein a “probe” refers to an oligonucleotide sequence that may or may not be extended in the amplification reaction by a DNA polymerase. Probes that are very specific for a perfectly complementary target sequence and strongly reject closely related sequences having one or a few mismatched bases are known in the art as “allele discriminating”. Probes that hybridize under at least one applicable detection condition not only to perfectly complementary sequences, but also to partially complementary sequences having one or more mismatched bases, are “mismatch tolerant” probes.

As used herein an “oligonucleotide set”, “primer set” or “probe set” refers to a collection of primers or primers and probes for performing amplification or sequencing reactions. Another analogous term is “oligonucleotide library”, “primer library”, or “probe library”. In some embodiments, methods of assembling libraries containing nucleic acids, primers, and probes having predetermined sequence variations are provided herein. Assembly strategies provided herein can be used to generate very large libraries representative of many different nucleic acid probe sequences of interest. In some embodiments, libraries of nucleic acids are libraries of sequence variants. Sequence variants may be variants of a single naturally-occurring sequence. However, in some embodiments, sequence variants may be variants of a plurality of different sequences. A high-density nucleic acid library may include more than 100 different sequence variants (e.g., about 10² to 10³; about 10³ to 10⁴; about 10⁴ to 10⁵; about 10⁵ to 10⁶; about 10⁶ to 10⁷; about 10⁷ to 10⁸; about 10⁸ to 10⁹; about 10⁹ to 10¹⁰; about 10¹⁰ to 10¹¹; about 10¹¹ to 10¹²; about 10¹² to 10¹³; about 10¹³ to 10¹⁴; about 10¹⁴ to 10¹⁵; or more different sequences) wherein a percentage of the different sequences are specified sequences as opposed to random sequences (e.g., more than about 10, 15, 20, 25, 30, 35, 40, 45, 50, 55, 60, 65, 70, 75, 80, 85, 90, 91, 92, 93, 94, 95, 96, 97, 98, 99% of the sequences are predetermined sequences of interest). In some embodiments, the libraries may contain information obtained from sequencing reactions, such as target nucleic acid sequences, sequence reads, sequence alignments, bisulfite-converted sequences, methylation frequencies, allele frequencies, single nucleotide variants or polymorphisms, among others. Libraries may be stored or kept on high-density arrays, microarrays, microchips, computer-readable media, or by any method or apparatus known in the art.

In some embodiments, the oligonucleotides, primers, and probes may comprise universal (common to all oligonucleotides), semi-universal (common to at least of portion of the oligonucleotides) or individual or unique primer (specific to each oligonucleotide) binding sites (also referred to herein as “priming sites”) on either the 5′ end or the 3′ end or both. As used herein, the term “universal” primer or primer binding site means that a sequence used to amplify the oligonucleotide is common to all oligonucleotides such that all such oligonucleotides can be amplified using a single set of universal primers. In other circumstances, an oligonucleotide contains a unique primer binding site. As used herein, the term “unique primer binding site” refers to a set of primer recognition sequences that selectively amplifies a subset of oligonucleotides. In yet other circumstances, an oligonucleotide contains both universal and unique amplification sequences, which can optionally be used sequentially.

A “linker” or “linker sequence” is a structure, which can be a unique nucleic acid sequence, that joins one molecule to another, such as attachment of a probe as described herein to another molecule or a substrate, wherein one portion of the linker is operably linked to a substrate, and wherein another portion of the linker is operably linked to the probe. As used herein, a linker or linker sequence may refer to a common nucleic acid sequence that is preferably non-complementary to a target nucleic acid sequence. A library of oligonucleotides, probes or primers as described herein may each contain a single common linker sequence.

A “barcode” or “barcode sequence” as used herein refers to a short stretch of nucleotides in a particular order, and different barcodes are different combinations of nucleotides. A barcode may be of any length, but is preferably between 4 to 15, more preferably between 4 to 10, and most preferably between 4 and 8, such as 4, 5, 6, 7 or 8 nucleotides long. Ideally, the barcodes are designed such that they can be unambiguously called post-sequencing or post-amplification. The sequence of the barcode may be identical or different for each nucleic acid molecule in a particular sequencing run, or according to a number of different parameters, such as target nucleic acid origin, sequence reads derived from the 5′ strand or the 3′ strand, sequence length or molecular weight of a sequence read. Barcode sequences may be derived computationally or by hand. In some aspects, barcode sequences can be randomly generated using an iterative script program, such as Perl.

Oligonucleotides, primers, and probes may also contain one or more sites (“restriction sites”) for cleavage by restriction endonucleases (also known as “restriction enzymes”). Type 1 enzymes cut DNA at random far from their recognition sequences. Type II enzymes cut DNA at defined positions close to or within their recognition sequences. Type III enzymes are also large combination restriction-and-modification enzymes. They cleave outside of their recognition sequences and require two such sequences in opposite orientations within the same DNA molecule to accomplish cleavage. Type IV enzymes recognize modified, typically methylated DNA. Restriction endonucleases are commercially available and restriction site sequences are well known in the art (New England Biolabs, Beverly, Mass.). Any restriction site sequence may be included in oligonucleotides, primers, and probes as described herein.

Oligonucleotides, primers, and probes may be isolated from natural sources or purchased from commercial sources. Oligonucleotides, primers, and probes described herein may also be synthesized by any suitable method, e.g., standard phosphoramidite methods such as those described by Beaucage, S. L. and Caruthers, M. H. (1981) Tetrahedron Lett. 22: 1859-1862 or the triester method according to Matteucci, M. D. and Caruthers, M. H. (1981) J Am. Chem. Soc. 103(11): 3185-3191, or by other chemical methods using either a commercial automated oligonucleotide synthesizer or high-throughput, high-density array methods known in the art (see U.S. Pat. Nos. 5,602,244, 5,574,146, 5,554,744, 5,428,148, 5,264,566, 5,141,813, 5,959,463, 4,861,571 and 4,659,774, incorporated herein by reference in its entirety for all purposes). Pre-synthesized oligonucleotides may also be obtained commercially from a variety of vendors. Oligonucleotides, primers, and probes may be prepared using a variety of microarray technologies known in the art. Pre-synthesized oligonucleotide and/or polynucleotide sequences may be attached to a support or synthesized in situ using light-directed methods, flow channel and spotting methods, inkjet methods, pin-based methods and bead-based methods set forth in the following references: McGall et al. (1996) Proc. Natl. Acad. Sci. U.S.A. 93(24): 13555-13560; Synthetic DNA Arrays In Genetic Engineering, Vol. 20:111, Plenum Press (1998); Duggan, D. J. et al., (1999) Nat. Genet. 21 (Suppl 1): 10-14; Microarrays: Making Them and Using Them In Microarray Bioinformatics, Cambridge University Press, 2003; U.S. Patent Application Publication Nos. 20030068633 and 20020081582; U.S. Pat. Nos. 6,833,450, 6,830,890, 6,824,866, 6,800,439, 6,375,903 and 5,700,637; and PCT Application Nos. WO 04/031399, WO 04/031351, WO 04/029586, WO 03/100012, WO 03/066212, WO 03/065038, WO 03/064699, WO 03/064027, WO 03/064026, WO 03/046223, WO 03/040410 and WO 02/24597.

In the context of the subject matter described herein, references are made to melting temperatures (Tₘ) of oligonucleotides, primers, and probes. Tₘ means the temperature at which half of the subject material exists in double-stranded form and the remainder is single stranded. Generally, the Tₘ of a primer is a calculated value using any method knows in the art, particularly the “% GC” method (Wetmar, J. G. (1991) Crit. Rev. Biochem. Mol. Biol. 26: 227-259), the “2(A+T) plus 4(G+C)” method at a standard condition of primer and salt concentration, or methods described in Santa Lucia, J. (1998) Proc. Natl. Acad. Sci. 95(4): 1460-1465 and von Ahsen, N. et al., (2001) Clin. Chem. 47: 1956-1961.

The term “methylation” as used herein, denotes the type of chemical modification of nucleic acids that involves the addition of a methyl group, for example to the C5 carbon atom of the cytosine pyrimidine ring or to the N6 nitrogen atom of the adenosine purine ring, with the first option being particularly preferred. This modification can be inherited and subsequently removed without changing the original nucleic acid sequence. As such, it is part of the epigenetic code and the most well characterized epigenetic mechanism. Methylation is reversible: methyl-transferases catalyze the transfer of a methyl group from S-adenosyl-L-methionine to cytosine or adenosine residues. Polymerases such as DNA polymerases do not copy the methylated status during replication (reviewed, e.g., in Robertson, K. D. and Wolffe, A. P. (2000) Nat. Rev. Genet. 1(1): 11-19; Li, E. (2002) Nat. Rev. Genet. 3: 662-673; Bird, A. P. (2002) Genes Dev. 16: 6-21).

The term “CpG dinucleotide sites” (or “CpG sites”), as used herein, refers to regions of DNA where a cytosine nucleotide is located immediately adjacent to a guanine nucleotide in the linear sequence. “CpG” refers to cytosine and guanine separated by a phosphate (i.e., --C--phosphate--G--). The “CpG” notation is used to distinguish a cytosine followed by guanine from a cytosine base paired to a guanine. Regions of the DNA that have a higher frequency or concentration of CpG sites are known as “CpG islands”. “CpG islands” may also define a contiguous region of genomic DNA that satisfies the criteria of (1) having a frequency of CpG dinucleotides corresponding to an “observed/expected ratio” greater than 0.6; (2) having a “GC content” greater than 0.5; and (3) having a length of at least 0.2 kb (as described in Gardiner-Garden et al., (1987) J. Mol. Biol., 196: 262-282), with the exception that repeat regions matching these criteria are excluded (or masked). “CpG island fragment” or “CGI fragment” are used interchangeably to refer to a nucleic acid molecule fragment mapping to and containing at least part of a CpG island. The term “CpG target sequence” refers to a stretch of bases targeted to selectively enrich for CpG island fragments and/or other methylation informative GC-rich fragments. Many genes in mammalian genomes have CpG islands associated with the transcriptional start site (including the promoter) of the gene, which play a pivotal role in controlling gene expression.

Methylation at the C5 of cytosine has been found in bacteria, fungi, plant and mammalian genomes. Approximately 60-90% of CpG dinucleotides are methylated in most mammalian cell types. The CpG dinucleotides are not uniformly distributed in mammalian genomes. For example, sequence analysis of the human genome has estimated nearly 30,000 CpG islands, which accounts for about 0.7% of the genome. CpG dinucleotides in the remaining 99.3% of the genome are sparsely distributed. Because of the high cytosine-guanine frequency of CpG islands, it is possible to identify them without knowledge of the methylation pattern of the DNA.

In normal tissue, CpG islands are often unmethylated but a subset of islands becomes methylated during oncogenesis, cellular development, and various disease states. Hypermcthylation (i.e. an increased level of methylation) of CpG sites within the promoters of genes can lead to their silencing, a feature found, e.g., in a number of human cancers (for example the silencing of tumor suppressor genes). In contrast, the hypomethylation (i.e. a reduced level of methylation) of CpG sites has been associated with the over-expression of oncogenes within cancer cells (reviewed, e.g., in Robertson, K. D. and Wolffe, A. P. (2000) Nat. Rev. Genet. 1(1): 11-19; Li, E. (2002) Nat. Rev. Genet. 3: 662-673; Bird, A. P. (2002) Genes Dev. 16: 6-21; Klose, R. J. and Bird, A. P. (2006) Trends Biochem. Sci. 31: 89-97). Accordingly, there is great interest in determining the methylation status or profiles of promoters and CpG islands (CG1s) in various tissues, especially with regard to methylation differences accounting for altered patterns of expression in normal development and in various disease states which would greatly improve understanding of these processes and provide potential diagnostic markers and therapeutic targets for diseases (Berman, B. P. et al., (2009) Nat. Biotech., 27(4): 341-342).

The term “methylation assay” refers to any assay for determining the methylation status of one or more CpG dinucleotide sequences within one or more nucleic acid sequences. “Methylation frequency”, “methylation state” or “methylation status” refers to a determination of the presence or absence of 5-methylcytosine (“5-mCyt”) or any other methylation modification at one or a plurality of CpG dinucleotides within a target nucleic acid sequence by a methylation assay. The methylation status of a particular nucleic acid fragment or sequence can indicate the methylation state of every base in the sequence or can indicate the methylation state of a subset of the base pairs (e.g., whether the base is cytosine or 5-methylcytosine) within the sequence. Methylation states at one or more particular CpG methylation sites (each having two CpG dinucleotide sequences) within a nucleic acid sequence may include “unmethylated,” “fully-methylated” and “hemi-methylated” sites. Methylation status can also indicate information regarding regional methylation density within the sequence without specifying the exact location at the single nucleotide position level.

A “methylation profile” refers to a set of data representing the methylation states or methylation frequencies of one or more loci within a molecule of DNA from e.g., the genome of an individual or cells or tissues from an individual. The profile can indicate the methylation state of every base in an individual, can have information regarding a subset of the base pairs (e.g., the methylation state of specific promoters or quantity of promoters) in a genome, or can have information regarding regional methylation density or methylation frequency of one or more loci with or without specifying the exact location at the single nucleotide position level.

“Differential methylation” denotes a condition in which a particular candidate genomic locus is (at one or more nucleic acid sites comprised in its sequence) methylated in at least one target sample but unmethylated in at least one reference sample, or vice versa, in which a particular candidate genomic locus is (at one or more nucleic acid sites comprised in its sequence) unmethylated in the reference sample but methylated in the target sample. The determination of the differential methylation pattern or frequency of the one or more candidate genes/loci already includes the identification of the exact nucleic acid sites (i.e. sequence elements, genetic loci) comprised in the one or more candidate genes. Preferably, the nucleic acid sites comprised in the one or more candidate genes/loci that are differentially methylated are CpG dinucleotide sites.

Generally, the determination of the methylation pattern, methylation frequency, or methylation status of the one or more candidate genes/loci may be accomplished by any means known in the art. Preferably, methylation is determined by means of one or more methods selected from reverse-phase HPLC, thin-layer chromatography, SssI methyltransferases with incorporation of labeled methyl groups, the chloracetaldehyde reaction, differentially sensitive restriction enzymes, hydrazine or permanganate treatment (m5C is cleaved by permanganate treatment but not by hydrazine treatment), bisulfite sequencing, combined bisulfite-restriction analysis, pyrosequencing, methylation-sensitive single-strand conformation analysis (MS-SSCA), high resolution melting analysis (HRM), methylation-sensitive single nucleotide primer extension (MS-SnuPE), base-specific cleavage/MALDI-TOF, methylation-specific PCR (MSP), microarray-based methods, and MspI cleavage (reviewed, e.g., in Rein, T. et al. (1998) Nucl. Acids Res. 26: 2255-2264). Methods for detecting methylation status have been described in, for example U.S. Pat. Nos. 6,214,556, 5,786,146, 6,017,704, 6,265,171, 6,200,756, 6,251,594, 5,912,147, 6,331,393, 6,605,432, 5,786,146, 6,143,504, 6,596,493, 6,884,586, 6,300,071, and 7,195,870 and U.S. Patent Application Publication Nos. 20030148327, 20030148326, 20030143606, 20050009059, and 20060292564, each of which are incorporated herein by reference. Other array based methods of methylation analysis are disclosed in U.S. Patent Application Publication No. 20050196792. See also, Oakeley, E. J., (1999) Pharmacol. Ther. 84: 389-400; Fraga et al., (2002) BioTechniques 33: 632-649; and Dahl et al., (2003) Biogerontology 4: 233-250.

Methods for identifying methylation may be based on differential cleavage by restriction enzymes are used. Methylation-sensitive restriction analysis followed by PCR amplification or Southern analysis have been disclosed, for example, in Huang, T. H. et al. (1997) Cancer Res. 57: 1030-1034; Zuccotti, M. et al, (1993) Meth. Enzymol. 225: 557-567; Carrel, L. et al. (1996) Am. J Med. Genet. 64: 27-30; and Chang et al. (1992) Plant Mol. Biol. Rep. 10: 362-366.

In some aspects, enzymes that include at least one CpG dinucleotide in the recognition site may be used. Enzymes with a recognition site that includes the sequence CCGG include, for example, MspI, HpaII, AgeI, XmaI, SmaI, NgoMIV, Noel, and BspEI. Enzymes with a recognition site that includes the sequence CGCG include, for example, BstUI (CGCG, MSRE), MluI (ACGCGT, MSRE), SacII (CCGCGG, MSRE), BssHII (GCGCGC, MSRE) and NruI (TCGCGA, MSRE). NotI, BstZI, CspI and EagI have two CpGs in their recognition sites and cleavage is blocked by CpG methylation. Enzymes with a recognition site that includes the sequence GCGC include, for example, HinPlI, HhaI, AfeI, KasI, NarI, SfoI, BbeI, and FspI. Enzymes with a recognition site that includes the sequence TCGA include, for example, TaqI, ClaI (MSRE), BspDI (MSRE), PaeR7I, TliI, XhoI, SalI, and BstBI. For additional enzymes that contain CpG in the recognition sequence and for information about the enzyme's sensitivity to methylation, see, for example, the New England Biolabs catalog and web site. In some aspects two restriction enzymes may have a different recognition sequence but generate identical overhangs or compatible cohesive ends. For example, the overhangs generated by cleavage with HpaII or MspI can be ligated to the overhang generated by cleavage with TaqI. Some restriction enzymes that include CpG in the recognition site are unable to cleave if the site is methylated; these are methylation sensitive restriction enzymes (MSRE). Other enzymes that contain CpG in their recognition site can cleave regardless of the presence of methylation; these are methylation insensitive restriction enzymes (MIRE). A third type of enzyme cleaves only when the recognition site is methylated, and are referred to herein as methylation dependent restriction enzymes (MDRE). Examples of MIREs that have a CpG in the recognition sequence include, for example, BsaWI (WCCGGW), BsoBI, BssSI, MspI, and TaqI. Examples of MSREs, that include a CpG in the recognition site, include AatII, AciI, AclI, AfeI, AgeI, AscI, AvaI, BmgBI, BsaAI, BsaHI, BspDI, ClaI, EagI, FseI, PauI, HaeIII, HpaII, HinPII, MluI, NarI, NotI, NruI, PvuI, SacII, SalI, SmaI and SnaBI. In preferred aspects a pair of enzymes that have differential sensitivity to methylation and cleave at the same recognition sequence with one member of the pair being a MSRE and the other member being MIRE is used. Still other enzymes include BthCI, GlaI, HpaI, HinPlI, DpnI, MboI, ChaI and BstKTI.

Bisulfite sequencing is a commonly used method in the art for generating methylation data at single-base resolution. The term “bisulfite conversion” refers to a biochemical process for converting unmethylated cytosine residue to uracil or thymine residues, whereby methylated cytosine residues are preserved. “Bisulfite conversion” may be carried out computationally from a nucleic acid sequence contained in a computer file (such as those in FASTA, FASTQ or any file format known in the art), wherein all cytosine residues in a sequence of interest are changed to thymine or uracil residues. Exemplary reagents for bisulfite conversion include sodium bisulfite and magnesium bisulfite. “Bisulfite reagent” refers to a reagent comprising bisulfite, disulfite, hydrogen sulfite or combinations thereof, useful as disclosed herein to distinguish between methylated and unmethylated CpG dinucleotide sequences. One way to obtain such methylation data for the CG Is is to sequence the entire epigenome directly. Due to the difficulty in mapping bisulfite converted sequence reads and the methylation heterogeneity in a cell population, approximately 100 gigabases (Gb) of sequence data would be needed to generate a high-resolution human DNA methylation map (Lister, R. et al., (2009) Nature, 462(7271): 315-322). Other methylation profiling approaches include array capture (Hodges, E. et al., (2009) Genome Res. 19(9): 1593-1605), padlock probe capture (Deng, J. et al., (2009) Nat. Biotech. 27: 353-360; Ball, M. P. et al., (2009) Nat, Biotech., 27(4): 361-368) and reduced representation bisulfite sequencing (Gu et al., (2010) Nat. Methods 7(2): 133-136).

In particular, bisulfite sequencing involves conversion of unmethylated cytosine to uracil or thymine through a three-step process during sodium bisulfite modification. The steps are sulfonation to convert cytosine to cytosine sulfonate, deamination to convert cytosine sulfonate to uracil sulfonate or thymine sulfonate and alkali desulfonation to convert uracil sulfonate to uracil or thymine sulfonate to thymine. Conversion of methylated cytosine is much slower and is not observed at significant levels in a 4-16 hour reaction (Clark, S. J. et al, (1994) Nucleic Acids Res., 22(15): 2990-7). If the cytosine is methylated it will remain a cytosine. If the cytosine is unmethylated, it will be converted to uracil or thymine. When the modified strand is copied, through, for example, extension of a locus specific primer, a random or degenerate primer or a primer to an adaptor, a G will be incorporated in the interrogation position (opposite the C being interrogated) if the C was methylated and an A will be incorporated in the interrogation position if the C was unmethylated. When the double stranded extension product is amplified, those Cs that were converted to Us or Ts and resulted in incorporation of A in the extended primer will be replaced by Ts during amplification. Those Cs that were not modified and resulted in the incorporation of G will remain as C. Bisulfite treatment can degrade the DNA making it difficult to amplify. The sequence degeneracy resulting from the treatment also complicates primer design. The treatment may also result in incomplete desulfonation, depurination and other as yet uncharacterized DNA damage, making downstream processing more challenging. The treatment can also result in preferential amplification of unmethylated DNA relative to methylated DNA. This may be mitigated by increasing the PCR extension time.

Kits for DNA bisulfite modification are commercially available from, for example, Human Genetic Signatures' Methyleasy and Chemicon's CpGenome Modification Kit. See also, WO04096825, which describes bisulfite modification methods and Olek, A. et al. (1994) Nucl. Acids Res. 24(24): 5064-6, which discloses methods of performing bisulfite treatment and subsequent amplification on material embedded in agarose beads. In some aspects a catalyst such as diethylenetriamine may be used in conjunction with bisulfite treatment, see Komiyama, M. and Oshima, S., (1994) Tetrahedron Lett. 35(44): 8185-8188. Diethylenetriamine has been shown to catalyze bisulfite ion-induced deamination of 2′-deoxycytidine to 2′-deoxyuridine at pH 5 efficiently. Other catalysts include ammonia, ethylene-diamine, 3,3′-diaminodipropylamine, and spermine. In some aspects, deamination is performed using sodium bisulfite solutions of 3-5 M with an incubation period of 12-16 hours at about 50° C. A faster procedure has also been reported using 9-10 M bisulfite pH 5.4 for about 10 minutes at 90° C., see Hayatsu, H. et al., (2004) Proc. Jpn. Acad. Ser. B 80(4): 189-194.

Bisulfite treatment allows the methylation status of cytosines to be detected by a variety of methods. For example, any method that may be used to detect a single nucleotide polymorphism (SNP) may be used, for examples, see Syvanen, A. C. (2001) Nature Rev. Gen. 2(12): 930-942. In a preferred aspect, bisulfite sequencing methods, systems, and computer program products described herein may provide information regarding not only methylation frequencies or methylation status of a sequence of interest at single base resolution, but also information regarding SNPs, preferably in the same sequencing run. Other methods such as single base extension (SBE) may be used or hybridization of sequence specific probes similar to allele specific hybridization methods. “Variants” or “alleles” generally refer to one of a plurality of species each encoding a similar sequence composition, but with a degree of distinction from each other. The distinction may include any type of variation known to those of ordinary, skill in the related art, that include, but are not limited to, polymorphisms such as SNPs, insertions or deletions (the combination of insertion/deletion events are also referred to as “indels”), differences in the number of repeated sequences (also referred to as tandem repeats), and structural variations. Detection of such variants or alleles is also within the ambit of the subject matter described herein.

In a preferred aspect, molecular inversion probes (MIP), described in Hardenbol, P. et al. Genome Res. 15:269-275 (2005) and in U.S. Pat. No. 6,858,412, may be used to determine methylation status after methylation dependent modification. A MIP may be designed for each cytosine to be interrogated. In a preferred aspect the MIP includes a locus specific region that hybridizes upstream and one that hybridizes downstream of an interrogation site and can be extended through the interrogation site, incorporating a base that is complementary to the interrogation position. The interrogation position may be the cytosine of interest after bisulfite modification and amplification of the region and the detection can be similar to detection of a polymorphism. Separate reactions may be performed for each NTP so extension only takes place in the reaction containing the base corresponding to the interrogation base or the different products may be differentially labeled.

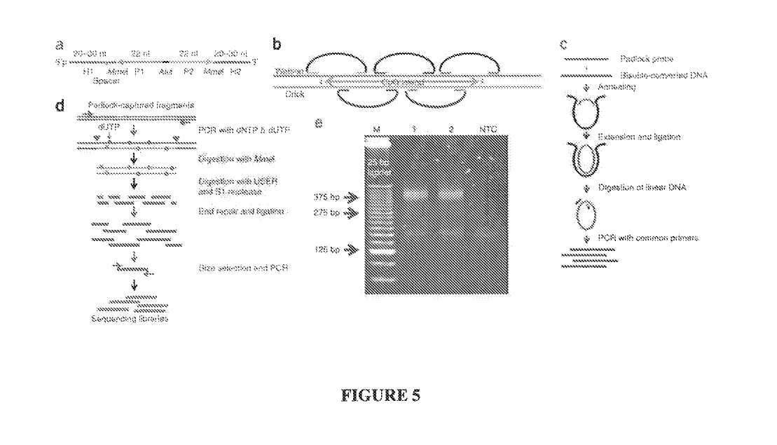

The term “padlock probe” (PLP) refers to circularized nucleic acid molecules which may combine specific molecular recognition and universal amplification (or specific amplification and general recognition), thereby increasing sensitivity and multiplexing capabilities without limiting the range of potential target organisms. PLPs are long oligonucleotides of approximately 100 bases (but can be of any length), containing target complementary regions (referred to herein as “target-capturing sequences”) at both their 5′ and 3′ ends (See, for example, FIG. 5). These regions recognize adjacent sequences on the target nucleic acid sequence (Nilsson, M., et al. (1994) Science 265: 2085-2088) and may also contain “binding arms” which comprise “extension arms” having priming sites (e.g., universal priming sites”), sites recognized by ligase enzymes, and unique sequence identifiers, sometimes referred to as a “ZipCode” or “barcode”. Upon hybridization, the ends of the probes are situated into adjacent position, and can be joined by enzymatic ligation at the ligation sites (also referred to herein as “ligation arms”) converting the probe into a circular molecule (also known in the art and referred to herein as an “amplicon”) that is threaded on the target strand. This ligation and the resulting circular molecule can only take place when both ligation arm and extension arm segments recognize their target sequences correctly. Non-circularized probes may be removed by exonuclease treatment, while the circularized entities may be amplified with universal primers, which may or may not contain barcode or ZipCode sequences. This mechanism ensures reaction specificity, even in a complex nucleotide extract with a large number of padlock probes. Subsequently, the target-specific products are detected by a universal cZipCode microarray (Shoemaker, D. D., et al., (1996) Nat. Genet. 14: 450-456). PLPs have high specificity and multiplexing capabilities in gcnotyping assays (Hardcnbol, P., et al., (2003) Nat. Biotechnol., 21: 673-678.).

A “formula,” “algorithm,” or “model” is any mathematical equation, algorithmic, analytical or programmed process, or statistical technique that takes one or more continuous or categorical inputs (herein called “parameters”) and calculates an output value, sometimes referred to as an “index” or “index value.” Non-limiting examples of “algorithms” include sums, ratios, and regression operators, such as coefficients or exponents, value transformations and normalizations, rules and guidelines, statistical classification models, pattern recognition, linear and quadratic discrimination, support vector machines, principal component analysis, nearest neighbor search, naïve Bayesian classifiers, and neural networks.

A “neural network” can be an Artificial Neural Network (ANN) and are information processing systems composed of varying numbers of simple elements called neurons distributed into layers. Neurons are organized in an input layer, one or more hidden layers, and an output layer. The connections between elements determine network function just as in natural biological nervous systems. ANN is an intelligent technique that mimics the functioning of a human brain, and emulates human intuition of making decisions and drawing conclusions even when presented with complex, noisy, irrelevant and partial information. ANNs may have any number of hidden layers. The neurons are connected to each other by weighted links over which signals can pass. Each neuron receives multiple inputs from other neurons, except the neurons in the input layer, in proportion to their connection weights and then generates a single output in accordance with an activation function. An activation function can be linear or nonlinear depending on the application. Sigmoid or Hyperbolic Tangent activation function can be used to improve the performance of ANNs in power system applications.

An ANN can be trained to perform a particular function by adjusting values of the interconnections called weights, and neuron thresholds. The process of adjusting interconnection weights and neuron thresholds to achieve output of the ANN the same as the target value or desired output for a given input is referred to as “training” of ANN. Training an ANN consists of adjusting interconnection weights of neurons using a learning algorithm. Back propagation with momentum is the commonly used learning algorithm. Multilayer Feed Forward ANNs with Error Back Propagation learning algorithm are also commonly used, Feed Forward calculations, and propagating error from output layer to input layer and weight updating in hidden and output layers are major steps of training algorithm.

By way of example, the neural networks described herein comprise one or more inputs that are associated with efficiency of the probe or primer described herein. By “efficiency” is meant the amount of target nucleic acid sequence represented by a particular probe or primer in a sequencing library. Standard methods can be used to calculate the efficiency by measuring or counting the amount of target nucleic acid(s) and the amount of unbound target nucleic acid(s) via sequencing. The efficiency of a probe or primer described herein is typically compared to the capture efficiency of a control probe or primer under the same incubation conditions (e.g., using same buffer and temperature).

Such inputs comprise, without limitation, target length, target folding energy, target GC content, extension arm A %, extension arm G %, target A %, target T %, target G %, number of “GG” dinucleotides in ligation arm, number of “AT” dinucleotides in extension arm, number of “GG” dinucleotides in extension arm, number of “AA” dinucleotides in target, number of “AT” dinucleotides in target, number of “TA” dinucleotides in target, number of “GT” dinucleotides in target, number of “GA” dinucleotides in target, ligation arm terminal dinucleotide, extension arm terminal dinucleotide, target 5′ terminal dinucleotide, ligation arm melting temperature, extension arm melting temperature, ligation arm length, extension arm length, local single-stranded folding energy of the target, and the dinucleotides present at the extension site and ligation site during probe capture. The neural networks described herein may comprise one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five or more inputs.

The methods, computer program products, and systems described herein may comprise one, two, three, four, five or more neural networks. In a preferred embodiment, the probe designing algorithm used to predict efficiency of probes or primers as described herein utilizes two neural networks.

In some exemplary embodiments, there is provided methods, systems, and computer program products for designing large numbers of efficient, low-bias padlock probe molecules using a computational algorithm for probe or primer efficiency prediction. For example, a process may be implemented as a software application or computer program product (also known as software, programs, applications, components, or code) stored in memory and executed by one or more processors contained in a system. The software application or computer program product may accept a complete genome and a tabular list of specific regions of interest, along with several customizable probe properties (such as the inputs described herein, including, e.g., a desired target length, a desired binding arm length, and a desired DNA melting temperature). The software application or computer program product may use this information in an intelligent decision mechanism, such as for example a back propagation neural network-derived equation, to predict probe efficiency based on many probe characteristics. The software application or computer program product may output the optimal set of probes to obtain maximum coverage of a desired set of genomic regions in a high-throughput sequencing experiment. The software application or computer program product may also add a customized linker sequence to each probe molecule, allowing easy usage in modern high-throughput sequencers. Alternatively, the probe design software application may also perform an in silico bisulfite conversion prior to probe design in order to generate an optimal set of probes for targeted bisulfite sequencing. An additional feature may be implemented to allow the probe design software application or computer program product to output the most efficient padlock probe molecules for specific unique regions of a genome, allowing for efficient multiplex organism detection.

In some implementations, the methods, systems, and computer program products described herein may exhibit features, such as an increased number of targetable genomic regions for padlock probe sequencing, more efficient and unbiased capture of genomic regions, and reduced cost of sequencing to obtain target coverage. Moreover, the method, system, and computer program product described herein may extend to designing efficient padlock probes for SNP genotyping, mutation discovery, targeted genomic sequencing, quantification of allele specific gene expression, analysis of DNA methylation profiles or frequencies, and organism presence detection.

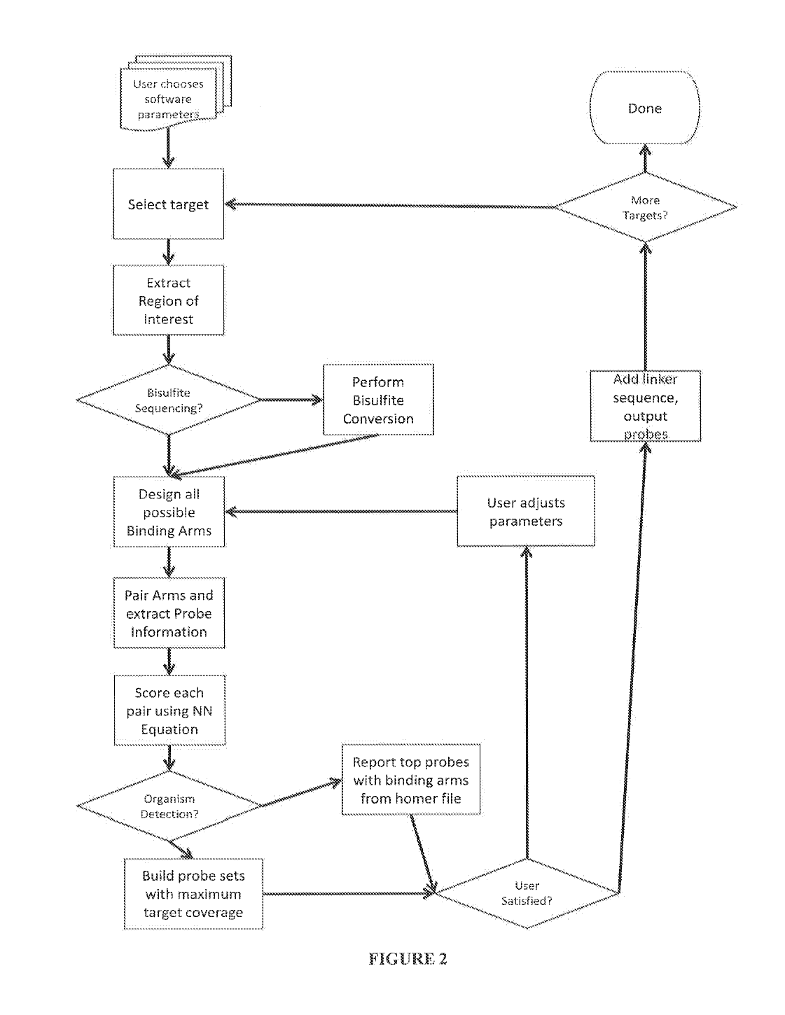

In some exemplary embodiments, the methods, systems, and computer program products described herein may be applied, in some implementations, with particular advantage to easily generate optimal, high-efficiency sets of padlock probes for targeted genomic sequencing experiments. FIG. 2 depicts a process 200 which may be implemented by a system comprising one or more processors and at least one memory including code which when executed by a processor provide the one or more of the operations depicted at process 200. For example, the process 200 may comprise a software application or computer program product implemented on at least one processor and at least one memory. In this example, the software application or computer program product may read three files presented by the user, such as for example, a job file, a target file, and/or a genome or chromosome file. Parameters or inputs provided in these files are associated with probe or primer efficiency and can be used to design padlock probes. The system may optionally comprise one or more databases containing libraries of information related to sequencing data, such as raw sequence reads, sequence alignments, methylation status or frequencies of a target nucleic acid sequence, and information regarding SNPs, allele frequencies, or other variations in nucleic acid sequences of interest. The system may also comprise one or more communication links connecting the one or more processors to the one or more databases.

The job file may include several customizable parameters or inputs, such as for example a range for desired binding arm size, a range for target region size, and/or a flag for whether single stranded folding energy of each target should be calculated using an external module. The job file may also include links to one or more software modules to be used and the location on disk of the software, target file, and/or genome/chromosome file.

The genome/chromosome file may include the genome or chromosome on which targeted sequencing is to be performed. The file may be configured in a FASTA or FASTQ format. In FASTA, an organism or chromosome name may be presented, beginning with the “>” character; example: “>chr1” may be the first line of a FASTA or FASTQ file. The subsequent lines may contain a string of DNA base characters (A, T, G, and C) or DNA ambiguity characters as determined by IUPAC (M, R, W, S, Y, K, V, H, D, B, or N). Line break characters may be allowed to enable easier reading. Multi-chromosome organisms (such as, for example, humans) may still require one “>” character per file; multiple chromosomes may be split into separate files (for example “human_chr1.fa” or “human_chr2.fa”).

The target file may include a tab-delimited list of specific genomic regions or target nucleic acid sequences to be sequenced with padlock probes. The first column in the file may include a specific identifier chosen (e.g., by an experimenter) to describe the target nucleic acid molecule, for example the name of a specific gene or specific regulatory region of interest. The second column may list the chromosome or genome for the specific targeted region. The third and fourth columns may list the beginning and end of the specific target region, in linear bases; the distance spanned can be as short as a single base pair (for genotyping) or thousands of base pairs (for larger-scale sequencing). The fifth column (which may not be included in the file) may accept a strand designation; if one is provided, only probes targeting one specifically chosen strand of DNA (either forward or reverse) may be designed; otherwise, probes may be designed utilizing both strands in combination to provide optimal capture.

The software application or computer program product implemented on one or more processors and at least one memory may read in the specified job file to obtain desired probe parameters or inputs. The software application or computer program product may then proceed sequentially through the target file, designing optimal probe sets for each target nucleic acid molecule. The software application or computer program product may begin by opening the first specified genome/chromosome and loading, for example, the entire sequence into random access memory for rapid access. The software application or computer program product may then extract the specific target region of interest or target capture sequence, along with flanking sequence both linearly upstream and downstream of the target, from the genome.

The software application or computer program may design most, if not all, possible binding arms (which include extension arms and ligation arms as defined herein) for a given target nucleic acid molecule or region using the extracted sequence. The software application or computer program product may take into account the desired binding arm size from the job file, and remove from consideration any very low-complexity binding arms (i.e., more than six nucleotides of the same kind in a row). The software application or computer program product may next associate each binding arm with every possible partner binding arm, taking into account the desired target region length provided in the job file. These arm pairs would represent the two sequences surrounding the common linker. The software application or computer program product may designs arm pairs using both the forward and reverse strand of the provided genome unless the target file specifies a chosen strand; in this case, arm pairs will only be generated for the designated strand.

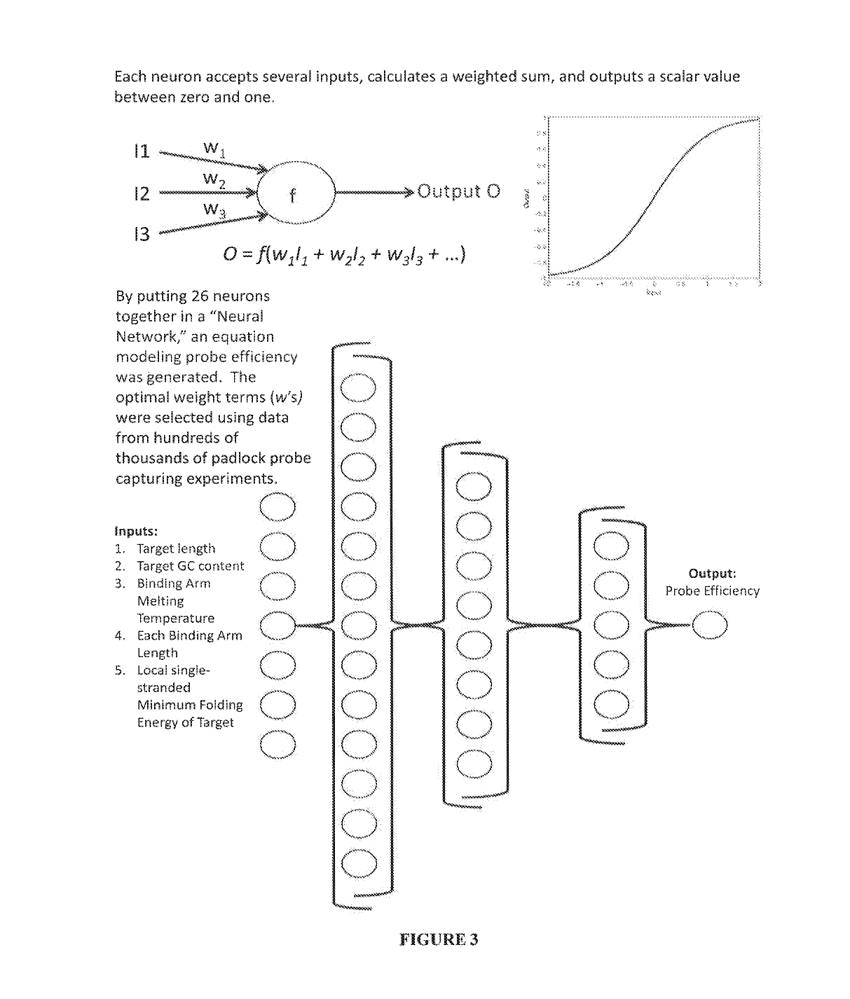

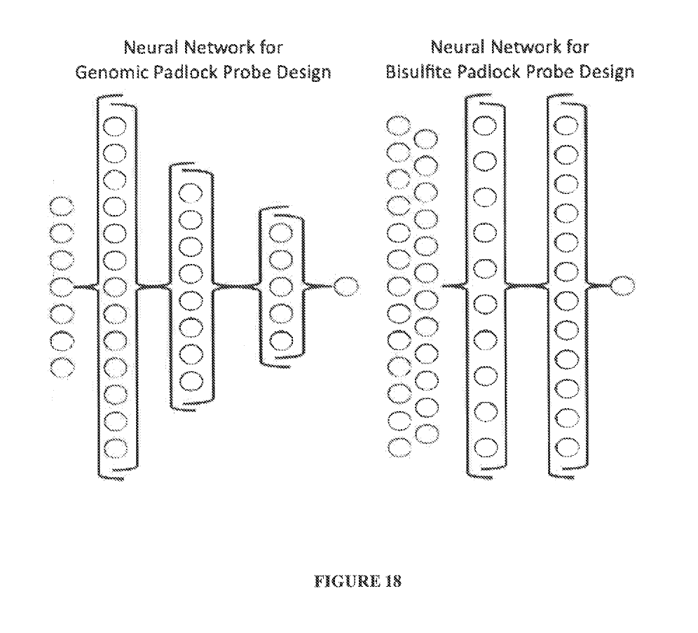

The software application or computer program product may then obtains a portion of information or inputs (e.g., six to seven key pieces of information) about each binding arm pair, such as for example the target length, the target GC content, the melting temperature of each binding arm, the length of each binding arm, and/or optionally the local single-stranded folding energy of the target. The software application or computer program product may then use a previously developed equation to predict the probe efficiency. This equation may be developed by an intelligent decision mechanism, such as a neural network, pattern recognizer, and/or other numerical techniques. For example, the equation to predict the probe efficiency may be developed by performing a multiplex genome sequencing reaction using hundreds of thousands of separate padlock probe molecules; the capturing efficiency of each molecule is measured in this sequencing experiment and modeled using one or more neural networks, including for example a back propagation neural network (with three layers containing 13, 8, and 5 nodes respectively, see FIG. 3) to the aforementioned 6-7 pieces of information as input. Additional neural networks may be added (such as, e.g., a network with two hidden layers having 10 and 12 nodes, see FIG. 18) with at least 25 pieces of information as input. The software application or computer program product may then divide each probe into separate categories based on which regions of the target sequence are captured, and ranks each available probe by probe efficiency. The probes having the highest efficiency are extracted and included in the generation of a library or probe set. The non-extracted probes of lower efficiency can be pooled and resubmitted for additional rounds of probe design as defined by the methods described herein.

The software application or computer program product may then generate candidate “probe sets” or “libraries” from the list of valid probes. The software application or computer program product may consider all possible sets of probe sets, and choose the set that provides maximum coverage of the sample and the highest aggregate probe scores, under the constraint that no binding arms in a probe set can bind to the same sequence (in order to prevent probe competition for hybridization). The software application or computer program product may also penalize sets that require many probes in order to control the cost of sequencing; this parameter may be adjustable.

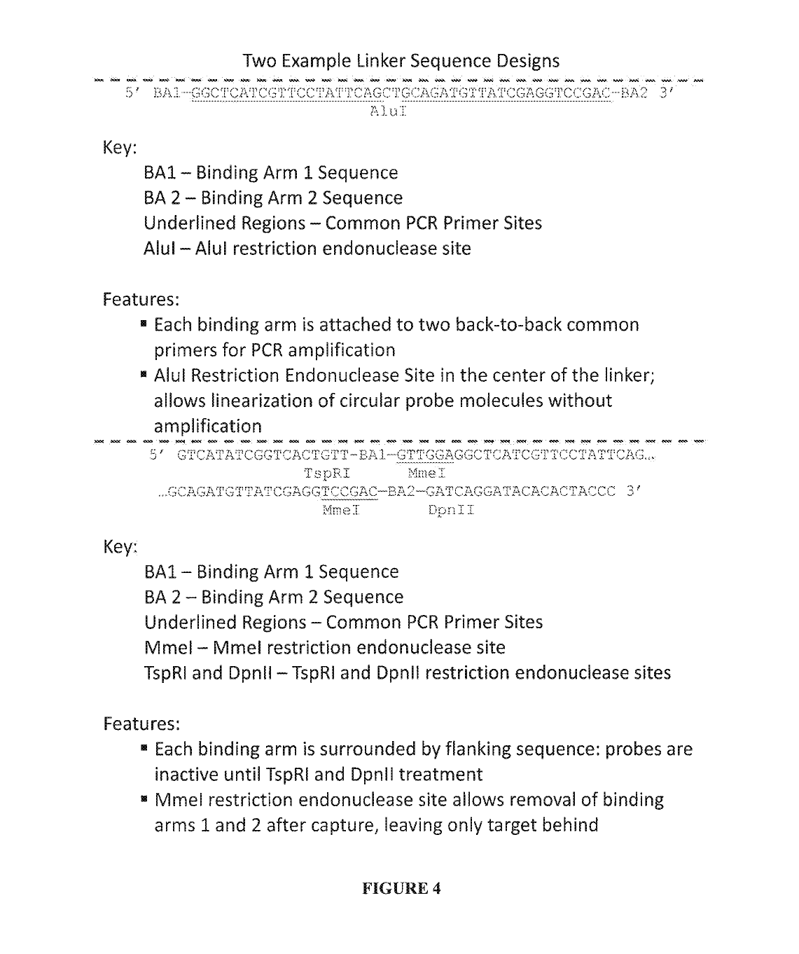

The software application or computer program product may then report all binding arm pairs for the optimal probe set to the user. If the user is satisfied, the software application may then attach a common linker sequence between the two binding arms and generate a single molecular sequence for each probe that can be generated using commercial DNA synthesis services (such as that provided by Integrated DNA Technologies) or via other high-throughput methods (see, e.g., U.S. Patent Application Ser. No. 60/765,978). Each linker sequence may be customizable to include specific adaptor sequences for current high-throughput sequencers, allowing designed padlock probes to be converted into linear sequencing libraries in just one experimental step. Linker sequences can also be customized to include barcode sequences for even greater multiplex capability or restriction enzyme sites for easy linearization of the circular padlock probe modules (see, e.g., FIG. 4). The user may thus integrate the generated probes into many custom experimental environments.

Once an optimal probe set is designed, the software application or computer program product may output it and proceed to the next target listed in the target file. The software application or computer program product may then repeat the genomic loading and probe design process. In some embodiments, the software application or computer program product may exclude probes having the highest efficiency and pool the remaining non-extracted probes and repeat the steps of generating a library or set of probe or primer sequences, determining efficiency of the probes in the generated library or set, and ranking the probe or primer sequences in the library or set by efficiency.

In some exemplary embodiments, the methods, systems, and computer program products described herein are related to characterizing the DNA methylation profile of a sample using bisulfite sequencing and include mapping a genomic sequence of interest to determine methylation status, methylation frequency, and detection of single nucleotide polymorphisms. During bisulfite sequencing, all cytosines present in the DNA molecule except those that are methylated are converted to thymines. Almost every methylated cytosine is present as a cytosine-guanine dinucleotide (CpG), though not all CpGs are methylated. In such embodiments, after the genome/chromosome is loaded into memory, the software application or computer program product performs an in silico bisulfite conversion, computationally converting all cytosines except those present in a CpG to thymines. The application or program then designs probes as previously, but penalizes the inclusion of CpG sites in each binding arm. During linker sequence insertion, the software application or computer program product generates multiple probes for those arm pairs containing a CpG; one probe assumes a methylated state (and contains a CpG dinucleotide) while the other assumes an unmethylated state (and contains a TpG instead). This procedure allows for efficient targeted bisulfite sequencing of hundreds of thousands of CpG sites in parallel.

In some exemplary embodiments, the methods, systems, and computer program products described herein use the probes or primers to obtain sequence reads of a target genome or sequence of interest by bisulfite sequencing and loading it into memory. A software application or computer program product encodes the sequence reads by predicting the forward and reverse orientation of each of the sequence reads to generate at least one forward sequence read and at least one reverse sequence read. The forward and reverse sequence reads are then converted by the software application or computer program product by computationally changing all cytosine residues in the forward sequence reads to thymine residues in silica, and changing all guanine residues to adenine residues in the reverse sequence reads. The bisulfite-converted genome sequence and all forward and reverse sequence reads are then aligned computationally by an alignment software application or computer program (e.g., ELAND, SOAP2Align, Bowtie, BWA, BLAST or any other alignment program known in the art). The alignment application or program can be a stand-alone application or integrated into the system, software application or computer program product described herein. The aligned sequences are then combined to create a map of the target genomic sequence. The software application or computer program product then analyzes and computes methylation frequencies or methylation status of the mapped sequences in entirety. In preferred embodiments, the mapped sequences may also be analyzed by the software application or computer program product for the presence of single nucleotide polymorphisms. Because bisulfite sequencing provides sequence read information at single-base resolution, this technique (and modifications thereof described in the methods, systems, and computer program products described herein) is particularly advantageous for calculating methylation frequencies and detecting SNPs in a single sequencing reaction.

In some exemplary embodiments, the methods, systems and computer program products described herein are related to organism detection in a mixed sample. Many cellular samples are heterogeneous, and contain mixtures of organisms in unknown quantities; padlock probes can be used to detect which and how many organisms of each of a given type are present. In this example, the software application or computer program product may accept a fourth input file, known as a “homer file,” which contains lists of preferred arm sequences in FASTA format (generally those found via genome annotation to be unique to a given genome or chromosome). The job file also contains at least one additional parameter: the number of probes to generate per genome or chromosome. The software application or computer program product then designs binding arm pairs as previously described, but favors binding arms containing a user-provided homer sequence. Instead of creating a “probe set” to maximize coverage of a target region, the software application or computer program instead returns the user-specified number of probes (in order of decreasing capturing efficiency) per genome. Probes designed for many separate genomes can be combined into a single padlock probe reaction, allowing detection of multiple organisms present at low frequency in a mixed population.

The subject matter described herein may be embodied in systems, computer program products, and methods, depending on the desired configuration. For example, the control module may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These software applications or computer program products include machine instructions for a programmable processor, and may be implemented in a high-level procedural and/or object-oriented programming language, and/or in assembly/machine language. As used herein, the term “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium.

A computer may include any type of computer platform such as a workstation, a personal computer, a server, or any other present or future computer. Computers typically include known components such as a processor, an operating system, system memory, memory storage devices, input-output controllers, input-output devices, and display devices. Many possible configurations and components of a computer exist in the art and may also include cache memory, a data backup unit, and other additional devices.

Display devices may include display devices that provide visual information, this information typically may be logically and/or physically organized as an array of pixels. An interface controller may also be included that may comprise any of a variety of known or future software programs for providing input and output interfaces. For example, interfaces may include “Graphical User Interfaces” (often referred to as GUI's) that provide one or more graphical representations to a user. Interfaces are typically enabled to accept user inputs using means of selection or input known to those of ordinary skill in the related art.

In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include a “shell” such as Unix Shells known to those of ordinary skill in the related art, or Microsoft Windows Powershell that employs object-oriented type programming architectures such as the Microsoft .NET framework. Interfaces may include one or more GUI's, CLI's or a combination thereof.

A processor may include a commercially available processor such as an Itanium® or Pentium® processor made by Intel Corporation, a SPARC® processor made by Sun Microsystems, an Athlon™ or Opteron™ processor made by AMD corporation, or it may be one of other processors that are or will become available. Some embodiments of a processor may also include Multi-core processors and/or employ parallel processing technology in a single or multi-core configuration. For example, a multi-core architecture typically comprises two or more processor “execution cores”. Each execution core may perform as an independent processor that enables parallel execution of multiple threads. In addition, a processor may be configured in what is generally referred to as 32 or 64 bit architectures, or other architectural configurations now known or that may be developed in the future.

A processor typically executes an operating system, which may be, for example, a Windows®-type operating system (such as Windows® XP, Windows Vista®, Windows 7) from the Microsoft Corporation; the Mac OS X operating system from Apple Computer Corp. (such as 7.5 Mac OS X v10.4 “Tiger” or 7.6 Mac OS X v10.5 “Leopard” operating systems); a Unix® or Linux-type operating system available from many vendors or an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

System memory may include any of a variety of known or future memory storage devices. Examples include any commonly available random access memory (RAM), magnetic medium such as a resident hard disk or tape, an optical medium such as a read and write compact disc, or other memory storage device. Memory storage devices may include any of a variety of known or future devices, including a compact disk drive, a tape drive, a removable hard disk drive, USB or flash drive, or a diskette drive. Such types of memory storage devices typically read from, and/or write to, a program storage medium such as, respectively, a compact disk, magnetic tape, removable hard disk, USB or flash drive, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with memory storage device.

In some embodiments, a computer program product is described comprising a computer usable medium having control logic (computer software program, including program code) stored therein. The control logic, when executed by a processor, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers could include any of a variety of known devices for accepting and processing information from a user, whether a human or a machine, whether local or remote. Such devices include, for example, modem cards, wireless cards, network interface cards, sound cards, or other types of controllers for any of a variety of known input devices. Output controllers could include controllers for any of a variety of known display devices for presenting information to a user, whether a human or a machine, whether local or remote. As presently described herein, the functional elements of a computer may communicate with each other via a system bus. Some embodiments of a computer may communicate with some functional elements using network or other types of remote communications.

As will be evident to those skilled in the relevant art, an instrument control and/or a data processing application, if implemented in software, may be loaded into and executed from system memory and/or a memory storage device. All or portions of the instrument control and/or data processing applications may also reside in a read-only memory or similar device of the memory storage device, such devices not requiring that the instrument control and/or data processing applications first be loaded through input-output controllers. It will be understood by those skilled in the relevant art that the instrument control and/or data processing applications, or portions of it, may be loaded by a processor in a known manner into system memory, or cache memory, or both, as advantageous for execution.

Also a computer may include one or more library files, experiment data files, and an internet client stored in system memory. For example, experiment data could include data related to one or more experiments or assays such as detected signal values, or other values associated with one or more sequencing experiments or processes. Additionally, an internet client may include an application enabled to accesses a remote service on another computer using a network and may for instance comprise what are generally referred to as “Web Browsers”. In the present example some commonly employed web browsers include Microsoft® Internet Explorer available from Microsoft Corporation, Mozilla Firefox® from the Mozilla Corporation, Safari from Apple Computer Corp., Google Chrome available from Google, Inc., or other type of web browser currently known in the art or to be developed in the future. An internet client may include, or could be an element of, specialized software applications enabled to access remote information via a network such as a data processing application for sequencing applications.

A network may include one or more of the many various types of networks well known to those of ordinary skill in the art. For example, a network may include a local or wide area network that employs what is commonly referred to as a TCP/IP protocol suite to communicate. A network may include a network comprising a worldwide system of interconnected computer networks that is commonly referred to as the internet, or could also include various intranet architectures. Some users in networked environments may prefer to employ what are generally referred to as “firewalls” (also sometimes referred to as Packet Filters, or Border Protection Devices) to control information traffic to and from hardware and/or software systems. For example, firewalls may comprise hardware or software elements or some combination thereof and are typically designed to enforce security policies put in place by users, such as for instance network administrators, etc.

The subject matter described herein may also make use of additional computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170. Additionally, the subject matter described herein may also make use of methods for providing genetic information over networks such as the Internet as shown in U.S. patent application Ser. Nos. 10/197,621, 10/063,559 (United States Publication No. 20020183936), Ser. Nos. 10/065,856, 10/065,868, 10/328,818, 10/328,872, 10/423,403, and 60/482,389.

The subject matter described herein also provides for design of oligonucleotides, primers, and probes useful for general sequencing reactions and assays. Commercial sequencing by synthesis platforms are available, such as the Genome Sequencer from Roche/454 Life Sciences, the Genome Analyzer from Illumina/Solexa, the SOLiD system from Applied BioSystems, Pacific Biosystems and the Heliscope system from Helicos Biosciences. Exemplary sequencing platforms may have one or more of the following features: 1) four differently optically labeled nucleotides are utilized (e.g., Genome Analyzer); 2) sequencing-by-ligation is utilized (e.g., SOLiD); 3) pyrosequencing is utilized (e.g., Roche/454); and 4) four identically optically labeled nucleotides are utilized (e.g., Helicos).

Such sequencing reactions and assays include sequencing by ligation methods commercialized by Applied Biosystems (e.g., SOLiD sequencing). In general, double stranded fragment nucleic acid molecules can be prepared by the methods described herein, and then incorporated into a water-in-oil emulsion along with polystyrene beads and amplified, for example by PCR. In some cases, alternative amplification methods can be employed in the water-in-oil emulsion such as any of the methods provided herein. The amplified product in each water microdroplet formed by the emulsion can interact, bind, or hybridize with the one or more beads present in that microdroplet leading to beads with a plurality of amplified products of substantially one sequence. When the emulsion is broken, the beads float to the top of the sample and are placed onto an array. The methods can include a step of rendering the nucleic acid bound to the beads single-stranded or partially single stranded. Sequencing primers are then added along with a mixture of four different fluorescently labeled oligonucleotide probes. The probes bind specifically to the two bases in the nucleic acid molecule to be sequenced immediately adjacent and 3′ of the sequencing primer to determine which of the four bases are at those positions. After washing and reading the fluorescence signal from the first incorporated probe, a ligase is added. The ligase cleaves the oligonucleotide probe between the fifth and sixth bases, removing the fluorescent dye from the nucleic acid molecule to be sequenced. The whole process is repeated using a different sequence primer until all of the intervening positions in the sequence are imaged. The process allows the simultaneous reading of millions of DNA fragments in a “massively parallel” manner. This “sequence-by-ligation” technique uses probes that encode for two bases rather than just one, allowing error recognition by signal mismatching and leading to increased base determination accuracy.

Other sequencing methods include sequencing by synthesis methods commercialized by 454/Roche Life Sciences including but not limited to the methods and apparatus described in Margulies et al., Nature (2005) 437:376-380 (2005); and U.S. Pat. Nos. 7,244,559; 7,335,762; 7,211,390; 7,244,567; 7,264,929; and 7,323,305. In general, double stranded fragment nucleic acid molecules can be prepared by the methods described herein, immobilized onto beads, and compartmentalized in a water-in-oil PCR emulsion. In some cases, alternative amplification methods can be employed in the water-in-oil emulsion such as any of the methods provided herein. When the emulsion is broken, amplified fragments remain bound to the beads. The methods can include a step of rendering the nucleic acid bound to the beads single stranded or partially single stranded. The beads can be enriched and loaded into wells of a fiber optic slide so that there is approximately 1 bead in each well. Nucleotides are flowed across and into the wells in a fixed order in the presence of polymerase, sulfhydrolase, and luciferase. Addition of nucleotides complementary to the target strand can result in a chemiluminescent signal that is recorded, such as by a camera. The combination of signal intensity and positional information generated across the plate allows software to determine the DNA sequence.

Other sequencing methods include those commercialized by Hclicos BioSciences Corporation (Cambridge, Mass.) as described in U.S. patent application Ser. No. 11/167,046, and U.S. Pat. Nos. 7,501,245; 7,491,498; 7,276,720; and in U.S. Patent Application Publication Nos. 20090061439; 20080087826; 20060286566; 20060024711; 20060024678; 20080213770; and 20080103058. In general, double stranded fragment nucleic acid molecules can be isolated and purified, then immobilized onto a flow-cell surface. The methods can include a step of rendering the nucleic acid bound to the flow-cell surface stranded or partially single stranded. Polymerase and labeled nucleotides are then flowed over the immobilized DNA. After fluorescently labeled nucleotides are incorporated into the DNA strands by a DNA polymerase, the surface is illuminated with a laser, and an image is captured and processed to record single molecule incorporation events to produce sequence data.

Other methods include sequencing by ligation methods commercialized by Dover Systems. Generally, oligonucleotides, primers, and probes can be prepared by the methods described herein. The nucleic acid molecules can then be amplified in an emulsion in the presence of magnetic beads. Any amplification methods can be employed in the water-in-oil emulsion. The resulting beads with immobilized clonal nucleic acid polonies are then purified by magnetic separation, capped, amine functionalized, and covalently immobilized in a series of flow cells. The methods can include a step of rendering the nucleic acid bound to the flow-cell surface stranded or partially single stranded. A series of anchor primers are flowed through the cell, where they hybridize to the synthetic oligonucleotide sequences at the 3′ or 5′ end of proximal or distal genomic DNA tags. Once an anchor primer is hybridized, a mixture of fully degenerate nonanucleotides (“nonamers”) and T4 DNA ligase is flowed into the cell. Each of the nonamer mixture's four components is labeled with one of four fluorophores, which correspond to the base type at the query position. The fluorophore-tagged nonamers selectively ligate onto the anchor primer, providing a fluorescent signal that identifies the corresponding base on the genomic DNA tag. Once the probes are ligated, fluorescently labeling the beads, the array is imaged in four colors. Each bead on the array will fluoresce in only one of the four images, indicating whether there is an A, C, G, or T at the position being queried. After imaging, the array of annealed primer-fluorescent probe complex, as well as residual enzyme, are chemically striped using guanidine HCl and sodium hydroxide. After each cycle of base reads at a given position have been completed, and the primer-fluorescent probe complex has been stripped, the anchor primer is replaced, and a new mixture of fluorescently tagged nonamers is introduced, for which the query position is shifted one base further into the genomic DNA tag. Seven bases are queried in this fashion, with the sequence performed from the 5′ end of the proximal tag, followed by six base reads with a different anchor primer from the 3′ end of the proximal tag, for a total of 13 base pair reads for this tag. This sequence is then repeated for the 5′ and 3′ ends of the distal tag, resulting in another 13 base pair reads. The ultimate result is a read length of 26 bases (thirteen from each of the paired tags). However, it is understood that this method is not limited to 26 base read lengths.

Other useful methods for sequencing include those commercialized by Illumina as described U.S. Pat. Nos. 5,750,341; 6,306,597; and 5,969,119. In general, oligonucleotides, primers, and probes can be prepared by the methods described herein to produce amplified nucleic acid sequences tagged at one (e.g., (A)/(A′) or both ends (e.g., (A)/(A′) and (C)/(C′)). In some cases, single stranded nucleic acid tagged at one or both ends is amplified by the methods described herein (e.g., by SPIA or linear PCR). The resulting nucleic acid is then denatured and the single stranded amplified nucleic acid molecules are randomly attached to the inside surface of flow-cell channels. Unlabeled nucleotides are added to initiate solid-phase bridge amplification to produce dense clusters of double-stranded DNA. To initiate the first base sequencing cycle, four labeled reversible terminators, primers, and DNA polymerase are added. After laser excitation, fluorescence from each cluster on the flow cell is imaged. The identity of the first base for each cluster is then recorded. Cycles of sequencing are performed to determine the fragment sequence one base at a time. For paired-end sequencing, such as for example, when the nucleic acid molecules are labeled at both ends by the methods described herein, sequencing templates can be regenerated in-situ so that the opposite end of the fragment can also be sequenced.

Still other sequencing methods include those commercialized by Pacific Biosciences as described in U.S. Pat. Nos. 7,462,452; 7,476,504; 7,405,281; 7,170,050; 7,462,468; 7,476,503; 7,315,019; 7,302,146; 7,313,308; and U.S. Patent Application Publication Nos. US20090029385; US20090068655; US20090024331; and US20080206764. In general, oligonucleotides, primers and probes can be prepared by the methods described herein. Target nucleic acid molecules can then be immobilized in zero mode waveguide arrays. The methods may include a step of rendering the nucleic acid bound to the waveguide arrays single stranded or partially single stranded. Polymerase and labeled nucleotides are added in a reaction mixture, and nucleotide incorporations are visualized via fluorescent labels attached to the terminal phosphate groups of the nucleotides. The fluorescent labels are clipped off as part of the nucleotide incorporation. In some cases, circular templates are utilized to enable multiple reads on a single molecule.

Another example of a sequencing technique that can be used in the methods described herein is nanopore sequencing (see e.g. Soni, G. V. and Meller, A. (2007) Clin. Chem. 53: 1996-2001). A nanopore can be a small hole of the order of one nanometer in diameter. Immersion of a nanopore in a conducting fluid and application of a potential across it can result in a slight electrical current due to conduction of ions through the nanopore. The amount of current that flows is sensitive to the size of the nanopore. As a DNA molecule passes through a nanopore, each nucleotide on the DNA molecule obstructs the nanopore to a different degree. Thus, the change in the current passing through the nanopore as the DNA molecule passes through the nanopore can represent a reading of the DNA sequence.

Another example of a sequencing technique that can be used is semiconductor sequencing provided by Ion Torrent (e.g., using the Ion Personal Genome Machine (PGM)). Ion Torrent technology can use a semiconductor chip with multiple layers, e.g., a layer with micro-machined wells, an ion-sensitive layer, and an ion sensor layer. Nucleic acids can be introduced into the wells, e.g., a clonal population of single nucleic can be attached to a single bead, and the bead can be introduced into a well. To initiate sequencing of the nucleic acids on the beads, one type of deoxyribonucleotide (e.g., dATP, dCTP, dGTP, or dTTP) can be introduced into the wells. When one or more nucleotides are incorporated by DNA polymerase, protons (hydrogen ions) are released in the well, which can be detected by the ion sensor. The semiconductor chip can then be washed and the process can be repeated with a different deoxyribonucleotide. A plurality of nucleic acids can be sequenced in the wells of a semiconductor chip. The semiconductor chip can comprise chemical-sensitive field effect transistor (chemFET) arrays to sequence DNA (for example, as described in U.S. Patent Application Publication No. 20090026082). Incorporation of one or more triphosphates into a new nucleic acid strand at the 3′ end of the sequencing primer can be detected by a change in current by a chemFET. An array can have multiple chemFET sensors.

Although a few variations have been described in detail above, other modifications or additions are possible. In particular, further features and/or variations may be provided in addition to those set forth herein. For example, the implementations described above may be directed to various combinations and subcombinations of the disclosed features and/or combinations and subcombinations of several further features disclosed above. In addition, the logic flow depicted in the accompanying figures and/or described herein does not require the particular order shown, or sequential order, to achieve desirable results. Other embodiments may be within the scope of the following claims.

### EXAMPLES

### Example 1

### Changes in DNA Methylation Detected by Targeted Bisulfite Sequencing

A method to specifically capture an arbitrary subset of genomic targets for single-molecule bisulfite sequencing and digital quantification of DNA methylation at single-nucleotide resolution is presented herein. A set of ˜30,000 padlock probes was designed to assess the methylation state of ˜66,000 CpG sites within 2,020 CpG islands on human chromosome 12, chromosome 20, and 34 selected regions. To investigate epigenetic differences associated with de-differentiation, methylation in three human fibroblast lines and eight human pluripotent stem cell lines was compared. Chromosome-wide methylation patterns were similar among all lines studied, but cytosine methylation was slightly more prevalent in the pluripotent cells than in the fibroblasts. Induced pluripotent stem (iPS) cells appeared to display more methylation than embryonic stem cells. In fibroblasts and pluripotent cells, 288 regions were methylated differently. This targeted approach is particularly useful for analyzing DNA methylation in large genomes.

Padlock probes have been previously used for exon capture and resequencing (Porreca, G. J. et al. (2007) Nat. Methods 4: 931-936). This approach to targeted bisulfite sequencing involves the in situ synthesis of long (˜150 nt) oligonucleotides on programmable microarrays, followed by their cleavage and enzymatic conversion into padlock probes. A library of padlock probes was annealed to the template DNA, circularized, and amplified by PCR before shotgun sequencing (FIG. 5A-5C). There are, however, two major challenges in performing padlock capture for bisulfite sequencing. First, bisulfite treatment converts all unmethylated cytosines into uracils, resulting in marked reduction of sequence complexity. Achieving specific target capture on bisulfite-converted DNA is more difficult than on native genomic DNA. Second, low capturing sensitivity, high bias and random losses of alleles was initially observed with the “eMIP method” previously disclosed in the art (Porreca, G. J. et al. (2007) Nat. Methods 4: 931-936). Obtaining accurate and efficient quantification of DNA methylation was not possible with the existing protocol, especially with the presence of allelic drop-outs.

### Materials and Methods

Padlock Probe Design

A probe design algorithm was developed to search for an optimal set of padlock probes covering an arbitrary set of non-repetitive genomic targets. This algorithm weights candidate probes based on several sequence features that were previously not considered in eMIP probe design, including the melting temperature, size and word statistics (distribution of 12-mers in the bisulfite-converted genome) of the capturing arms, and gap sizes. When the capturing arms contain one or more CpG dinucleotides, we used multiple probes to iterate all possible methylation state combinations of the CpGs contained within the arms. Chromosome positions of CpG islands were retrieved from the UCSC genome browser based on the hg18 annotation.

Padlock Probe Production

Libraries of long oligonucleotides (˜150 nt) were synthesized by ink-jet printing on a programmable microarray and released (Agilent Technologies). The estimated total yield is 10 fmol per library. PCR amplification was performed in 32-96 reactions (100 μl each) with 0.1 nM template oligonucleotides, 200 μM dNTPs, 400 nM Ap1V4IU primer, 400 nM Ap2V4 primer, 0.8× SybrGreen I, 36 units JumpStart Taq polymerase in 1× JumpStart buffer (Sigma), at 94° C. for 2 minutes, 22 cycles of 94° C. for 30 seconds, 55° C. for 2 minutes, 72° C. for 45 seconds and, finally, 72° C. for 5 minutes. The amplicons were purified by either column purification (Zymo DNA Concentrator—100 columns) or ethanol precipitation.

Approximately 40-60 μg of the purified PCR amplicons were digested with 40 units Lambda Exonuclease (5 U/μl; New England Biolabs (NEB)) in 1× lambda exonuclease buffer (NEB) at 37° C. for 2 hours, followed by denaturing at 90° C. for 5 minutes, and purified with six Qiagen Qiaquick PCR purification columns. The resulting single-stranded DNA was subsequently digested with 6 units USER enzyme (1 U/μl; NEB) in 1× DpnII buffer (NEB) at 37° C. for 4 hours. Ten μl of 100 μM DpnII_V4 guide oligo was added into the reaction and denatured the mixture at 95° C. for 5 minutes in a thermocycler, followed by a gradual decrease of temperature (0.1° C./s) to 60° C. and a 20-minute incubation at 60° C. The mixture was digested with 100 U DpnII (50 U/μl) at 37° C. for 2 hours. The single-stranded 102-nt probes were finally purified from the digestion with 6% denaturing PAGE (6% TB-Urea 2D gel; Invitrogen).

Multiplex Capture on Bisulfate-Converted DNA

Genomic DNA was extracted from frozen pellets of fibroblast, iPS or hES cells using Qiagen DNeasy columns and bisulfate converted with the Zymo DNA Methylation Gold Kit (Zymo Research). Padlock probes (60 nM) and 200 ng of bisulfite-converted genomic DNA were mixed in 10 μl 1× Ampligase Buffer (Epicentre), denatured at 95° C. for 10 minutes, then hybridized at 55° C. for 18 hours, after which 1 μl gap-filling mix (200 μM dNTPs, 2 U AmpliTaq Stoffel Fragment (ABI) and 0.5 units Ampligase (Epicentre) in 1× Ampligase buffer) was added to the reaction. For circularization, the reactions were incubated at 55° C. for 4 hours, followed by five cycles of 95° C. for 1 minute and 55° C. for 4 hours. To digest linear DNA after circularization, 2 μl exonuclease mix containing 10 U/μl exonuclease I and 100 U/μl exonuclease III (USB) was added to the reaction, and the reactions were incubated at 37° C. for 2 hours and then inactivated at 95° C. for 5 minutes.

Capture Circles Amplification

Ten microliters of circularization products were amplified by PCR in 100 μl reactions with 200 nM AmpF6.2-SoL primer, 200 nM AmpR6.2-SoL primer, 0.4× SybrGreen I and 50 μl iProof High-Fidelity Master Mix (Bio-Rad) at 98° C. for 30 seconds, eight cycles of 98° C. for 10 seconds, 58° C. for 20 seconds, 72° C. for 20 seconds, 14 cycles of 98° C. for 10 seconds, 72° C. for 20 seconds and 72° C. for 3 minutes. The amplicons of the expected size range (344-394 bp) were purified with 6% PAGE (6% TBE gel; Invitrogen).

Shotgun Sequencing Library Construction

Purified PCR products with the four probe sets on the same template DNA were pooled in equal molar ratio, and reamplified in 4×100 μl reactions with 4-μl template (10-15 ng/μl), 200 μM dNTPs, 20 μM dUTP, 200 nM AmpF6.3 primer, 200 nM AmpR6.3 primer, 0.4× SybrGreen I and 200 μl 2× Taq Master Mix (NEB) at 94° C. for 3 minutes, 8 cycles of 94° C. for 45 seconds, 55° C. for 45 seconds, 72° C. for 45 seconds and 72° C. for 3 minutes. PCR amplicons were purified with Qiaquick columns, and digested with MmeI: ˜3.6 nmole purified PCR amplicons, 16 units of MmeI (2 U/μl; NEB), 100 μM SAM in 1×NEB Buffer 4 at 37° C. for 1 hour. The digestions were again column purified, and digested with 3 U USER enzyme (1 U/μl) at 37° C. for 2 hours, then with 10 units S1 nuclease (10 U/μl; Invitrogen) in 1× S1 nuclease buffer at 37° C. for 10 minutes. The fragmented DNA was column purified, and end repaired at 25° C. for 45 minutes in 25-μl reactions containing 2.5 μl 10× buffer, 2.5 μl dNTP mix (2.5 mM each), 2.5 μl ATP (10 mM), 1 μl end-repair enzyme mix (Epicentre), and 15 μl DNA. Approximately 100-500 ng of the end-repaired DNA was ligated with 60 μM Solexa sequencing adaptors in 30 μl of 1× QuickLigase Buffer (NEB) with 1 μl QuickLigase for 15 minutes at 25° C. Ligation products of 150-175 bp in size were size selected with 6% PAGE, and amplified by PCR in 100 μl reactions with 15 μl template, 200 nM Solexa PCR primers, 0.8× SybrGreen I and 50 μl iProof High-Fidelity Master Mix (Bio-Rad) at 98° C. for 30 seconds, 12 cycles of 98° C. for 10 seconds, 65° C. for 20 seconds, 72° C. for 20 seconds and 72° C. for 3 minutes. The PCR amplicons were purified with Qiaquick PCR purification columns, and sequenced on Illumina Genome Analyzer. All primer sequences are listed in Table 1.

TABLE 1
Sequences of primers used in padlock capture and sequencing library
construction.

| Primer Name | Primer Sequence |
|---|---|
| AP1v41U | 5′-G*T*AGACTGGAAGAGCACTGTU-3′ |
| | (SEQ ID NO: 1) |
| AP2v4 | 5′-/Phos/TAGCCTCATGCGTATCCGAT-3′ |
| | (SEQ ID NO: 2) |
| DpnII_v4 | 5′-ATGCGTATCCGATC-3′ |
| | (SEQ ID NO: 3) |
| AmpF6.2Sol | 5′-AATGATACGGCGACCACCGACACTCTCTGCAGATGTTATCGAGGT-3′ |
| | (SEQ ID NO: 4) |
| AmpR6.2Sol | 5′-CAAGCAGAAGACGGCATACGAGCTCTTCACGCAGCTGAATAGGAACGAT-3′ |
| | (SEQ ID NO: 5) |
| AmpF6.3 | CAGATGTTATCGAGGTCCGAC |
| | (SEQ ID NO: 6) |
| AmpR6.3 | GGAACGATGAGCCTCCAAC |
| | (SEQ ID NO: 7) |

*indicates a phosphorothioate bond

Read Mapping and Data Analysis

Mapping of bisulfite sequencing reads was performed with SOAP (Li, R. et al. (2008) Bioinformatics 24: 713-714) driven by a customized Perl script. An unbiased mapping strategy in which the mapping success rate is independent of the methylation status was developed. Sequences of the captured targets were extracted from the repeat-masked human genome (hg18), and both strands were “bisulfite converted” in silico assuming no methylation on all CpG dinucleotides. The raw sequencing reads were also converted to “unmethylated reads”. To do this, the CIT and A/G ratios for each read were first compared to determine whether the reads corresponding to the bisulfite-converted strand or the reverse-complementary strand. In the latter case, the raw sequence was reverse complemented. All Cs were replaced by Ts in the resulting sequences. The unmethylated reads were then aligned to the unmethylated template sequences using SOAP. The false mapping rate that was due to the use of captured targets instead of the full human genome sequence was 0.21%. Finally, based on the mapping position, the methylation status of each CpG site was retrieved from the unconverted raw reads. Cluster analyses and statistical analyses were performed with R, Cluster3 Perl module, and in-house Perl scripts. The UCSC Genome Browser and Multiexperiment Viewer were used for data visualization.

### DISCUSSION

Approximately 10,580 padlock probes were designed, each capturing a 175- to 225-bp region, including 9,350 probes covering 2,020 CpG islands (Table 2) on human chromosomes 12 and 20, 705 probes covering 237 promoters in eight ENCODE (the Encyclopedia of DNA Elements) regions, and 527 probes targeting 4-kb regions centered on the transcription start sites (TSS) of 26 genes related to development or pluripotency (Table 3).

TABLE 2
Summary statistics of padlock captured CGIs

| | | Chromosome | Chromosome |
|---|---|---|---|
| | Total | 12 | 20 |
| CpG islands on the chromosome | 2020 | 1221 | 799 |
| CpG islands covered | 2020 | 1221 | 799 |
| CpG islands percentage covered* | 82% | 82% | 83% |
| CpG islands on promoter regions | 857 | 551 | 306 |
| CTG islands on gene body | 667 | 350 | 317 |
| CpG islands outside promoter and | 496 | 320 | 176 |
| gene body region | | | |

*Calculated as the fraction of base-pairs within CGIs that were covered by the padlock probes. Some CGIs were not completely covered due to the presence of repetitive sequences, or extreme nucleotide composition such that no probe could be designed.

TABLE 3
List of 26 selected genes and 8 ENCODE regions

| Target position | Gene |
|---|---|
| chr1: 063559560-63563560 | FOXD3 |
| chr1: 224193561-224197561 | LEFTY2 |
| chr10: 134891777-134895777 | UTF1 |
| chr11: 031787344-31791344 | PAX6 |
| chr12: 007831291-7835291 | NANOG |
| chr14: 056344940-56348940 | OTX2 |
| chr14: 100360210-100364210 | MEG3 |
| chr15: 022749222-22753222 | SNRPN |
| chr15: 022910785-22914785 | IPW |
| chr15: 047500765-47504765 | FGF7 |
| chr17: 037791988-37795988 | STAT3 |
| chr17: 042249080-42253080 | WNT3 |
| chr19: 001601591-1605591 | TCF3 |
| chr2: 066513974-66517974 | MEIS1 |
| chr20: 030811877-30815877 | DNMT3B |
| chr3: 055488366-55492366 | WNT5A |
| chr3: 057207043-57211043 | HESX1 |
| chr3: 171556146-171560146 | SKIL |
| chr3: 182910526-182914526 | SOX2 |
| chr4: 054788195-54792195 | PDGFRA |
| chr4: 057466834-57470834 | REST |
| chr4: 123965397-123969397 | FGF2 |
| chr5: 149524548-149528548 | CDX |
| chr5: 153836005-153840005 | HAND1 |
| chr6: 031244421-31248421 | OCT4 |
| chrX: 136474004-136478004 | ZIC3 |
| chr5: 153836005-153840005 | HAND1 |
| chr22: 3033954-31833953 | ENm004 |
| chr21: 32668237-34364221 | ENm005 |
| chrX: 152767492-154063081 | ENm006 |
| chr19: 59023585-60024460 | ENm007 |
| chr7: 26924046-27424045 | ENm010 |
| chr11: 1699992-2306039 | ENm011 |
| chr12: 38626477-39126476 | ENr123 |
| chr20: 33304929-33804928 | ENr333 |

The total size of captured fragments was 2.1 Mbp, representing 0.064% of the human genome. Because some probes contain CpG sites within the capturing arms, all possible C/T combinations were iterated on these CpG sites, and a total of 30,000 nondegenerate probes were synthesized. CpG islands were chosen to perform the proof-of-concept study primarily because they represent a relatively well-defined set of genomic features in the human genome annotation. To increase the sensitivity and reduce bias, the probe/target ratio was increased by more than tenfold, the reaction time was extended, and five additional cycles of circularization were added in comparison with the published protocol (Porreca, G. J. et al. (2007) Nat. Methods 4: 931-936). To integrate construction of sequencing libraries with padlock capture, a new method that uses a combination of uracil-specific excision reagent (USER) enzymes and S1 nuclease to create fragments with random ends was developed (FIG. 5D).

Validation of the targeted bisulfite sequencing method was achieved by capturing bisulfite-converted Jurkat cell genomic DNA with all 30,000 padlock probes in a single-tube reaction. PCR amplicons from the circularization reactions were template specific, and PAGE analysis showed the expected size distribution (FIG. 5E). The specificity of the capturing reaction was estimated by ligating captured DNA fragments to a sequencing vector, cloning these into Escherichia coli, and sequencing 96 clones. Of 89 high-quality Sanger sequencing reads obtained, 80 were from the targeted regions, indicating a specificity of 90%. An Illumina Genome Analyzer was then used to sequence the ends of the captured fragments. Approximately 5.5 million reads were mapped to 10,364 of 10,582 targets, which translates to a sensitivity of 98%. These results indicate that padlock probes can specifically extract a large set of genomic targets for single molecule bisulfite sequencing.

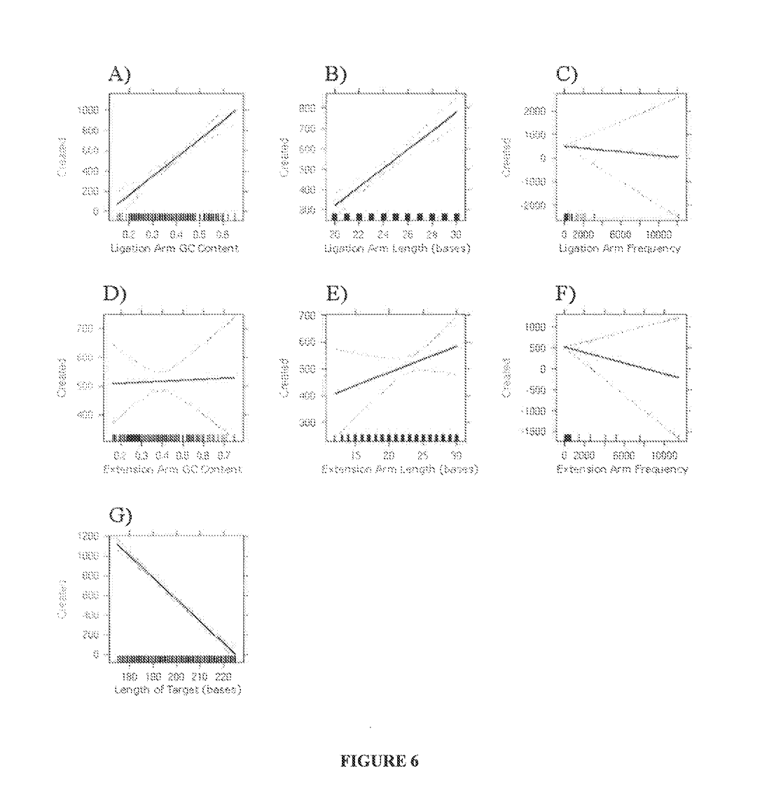

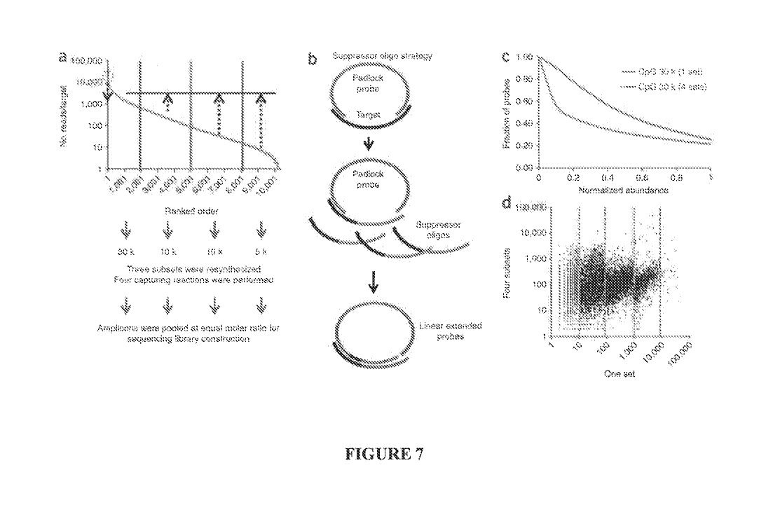

Although 98% of the targets were observed at least once in the end-sequencing analysis, the abundance of different captured fragments varied across a 10,000-fold range. Analysis of variance revealed that the bias resulted f′rom a combination of factors, including GC content and length of the ligation arms, and the size of the targets to be captured (FIG. 6). To normalize the relative abundance among different DNA fragments, a combination of two strategies was used: ‘subsetting’ and ‘suppressor oligos’ (FIG. 7A-7D). All 30,000 padlock probes were ranked based on the capturing efficiency determined by end sequencing, and divided into four subsets, two containing 5,000, and two containing 10,000, oligos. The three less efficient subsets were resynthesized. For each DNA sample, four capturing reactions were performed separately using probes from the original set of 30,000 and the three resynthesized subsets. The PCR amplicons from the capturing reactions were pooled in equal molar ratios before constructing a shotgun sequencing library (FIG. 7A). This subsetting strategy increased the relative abundance of less efficient targets by orders of magnitude. A very small number of probes were extremely efficient. For example, the top 48 (0.016%) most efficient probes account for 13.3% of mappable reads in the end-sequencing analysis.

Although the subsetting strategy allowed for adjusting the relative abundance among several relatively large subsets of probes, a method was also needed to specifically reduce the efficiency of a small number of probes in a library. For this, a set of 48 suppressor oligos was designed, which contained chimeric sequences: the 5′ region was reverse-complementary to the extension arm H2, and the 3′ region contained a short sequence unrelated to the ligation arm H1. When these suppressor oligos were mixed with padlock probes in a high molar ratio (100-fold molar excess of suppressor oligos), the 48 most efficient probes tended to anneal to the suppressor oligos, were extended from the 3′ ends and yielded linear-extended sequences that were removed in the subsequent exonuclease digestion (FIG. 7B). This normalization strategy was tested on the same bisulfite-converted Jurkat cell DNA, end sequencing on the captured DNA fragments was performed and 2.2 million mappable reads were obtained. The effect of normalization resulted in the fraction of probes with at least half of the average abundance increased from 31% to 49%; the average efficiency for the 48 most abundant probes was reduced by fivefold (FIG. 7C-7D).

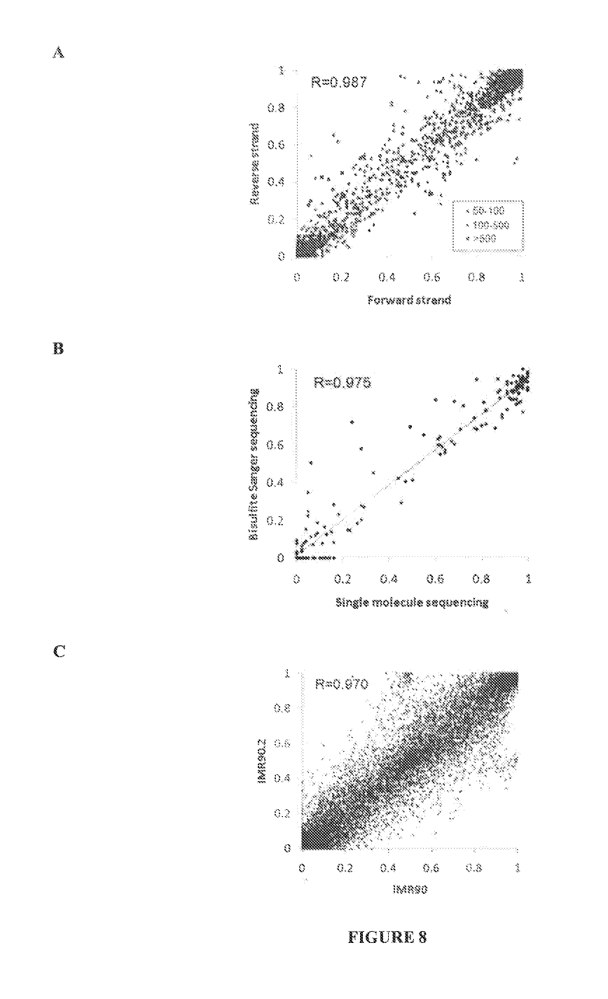

To validate the measurement accuracy of this method, advantage was taken of the built-in redundancy in our probe design. Each CpG island was covered by multiple probes targeting partially overlapping DNA fragments on alternating strands (FIG. 5B). The CpG sites in the overlapping regions were captured independently from two DNA strands with different probes. Because the sequencing reads were mapped in a strand-specific manner and CpG methylation is symmetric on the two DNA strands, the accuracy of the assay can be determined by comparing the methylation level of these CpG sites on the two strands. For 2,697 such CpG sites that were covered by >50 sequencing reads, the Pearson correlation coefficient (R) was 0.987 (FIG. 8A). To confirm the measurement accuracy with an independent method, the methylation levels of 182 randomly selected CpG sites were quantified with conventional bisulfite Sanger sequencing. Correlation between the two assays was high (R=0.975; FIG. 8B). Finally, the methylation measurements from two batches of IMR90 fibroblast cultures were compared. Correlation was observed between the biological replicates (R=0.970; FIG. 8C). Taken together, these three validation experiments indicate that this assay is highly robust.

To demonstrate the utility of targeted bisulfite sequencing, the changes of chromosome-wide methylation status during reprogramming of human fibroblasts to pluripotent cells was characterized. The methylation assay was performed on three sets of fibroblasts and iPS cells from three laboratories: IMR/IMR90-iPS17 reprogrammed with four factors (Oct4, Sox2, Nanog and Lin28); hFib2/hFib2-iPS18 reprogrammed with a different set of factors (Oct4, Sox2, K1f4 and Myc); BJ/BJ-iPS11/BJ-iPS12 (Maherali, N. et al. (2008) Cell Stem Cell 3: 340-345) reprogrammed with the five factors (Oct4, Sox2, Nanog, Klf4 and Myc) controlled by an inducible promoter. A line of hybrid stem cells (BJHues6-Hybrid1), which were reprogrammed by fusing the human fibroblasts (BJ) with hES cells (Hues6), as well as three hES cell lines (Hues 12, Hues42, Hues63) were also characterized. Bisulfite conversion, padlock capture and construction of shotgun sequencing libraries were performed on each DNA sample. Each library in one lane was sequenced in the flow cell of an Illumina Genome Analyzer, and yielding 2-3 million reads that were mapped to the targeted regions. The bisulfite conversion rates were >98.5%. To avoid stochastic sampling drift, CpG sites that were covered by <10 reads were removed from the following analyses.

The global methylation patterns in all 12 samples (11 cell lines plus a biological replicate on IMR90 fibroblasts) were visualized using the UCSC Genome Browser. The chromosome-wide patterns of CpG island methylation were highly similar among all the cell lines. Globally, the methylation level of CpG dinucleotides followed similar bimodal distribution: 67% were weakly methylated (<20% methylation), 22% were highly methylated (>80% methylation) and the remaining 11% had intermediate levels of methylation. To distinguish CpG islands with different methylation patterns, a histogram (bin size=0.05) for the distribution of methylation on all CpG sites within a CpG island was generated. Treating such histograms as 20-component vectors, hierarchical clustering was performed to partition the CpG islands and divide all CpG islands into three clusters based on the similarity of distribution between pluripotent and fibroblast lines. In cluster 1, the CpG islands (1,451; 77.3%) have similar distributions in the two cell types (R>0.5); in cluster 2, CpG islands (252; 13.4%) have less similar distributions (0.5≧R>0); in cluster 3, CpG islands (173; 9.2%) are anti-correlated (R<0). Therefore, only a small fraction of CpG islands show cell-type-specific methylation.

Because CpG islands are not defined in a functional manner, the CpG islands were divided into three categories. The first comprises CpG islands in the regions from 2 kb upstream to 500 bp downstream of TSS. These “upstream regions” often include promoter regions. The second class (“gene body CpG islands”) comprises CpG islands in the regions from 500 bp downstream of TSS to the ends of the last exons. The final category comprises CpG islands outside of gene body and promoter regions. CpG islands in each category were further divided into three groups according to CpG density. Consistent with previous findings, most (91.8%) CpG islands in promoter regions were weakly methylated (<20% methylation), 3.4% were highly methylated (>80% methylation) and the remaining 4.8% showed an intermediate level of methylation (20-80% methylation). The distributions were quite similar among the three groups with different CpG densities. In contrast, only 45.2% of CpG islands in the gene body were weakly methylated, whereas roughly one-third of them (37.7%) were highly methylated. Methylated CpGs tended to locate in islands with low CpG density. In regions outside of gene body and promoter regions, more weakly methylated CpG islands (58.9%) than highly methylated CpG islands (26.6%) were found. Similarly, CpG islands with low CpG density were more methylated. There were 80 genes in the data set that contained both promoter-region CpG islands and gene-body CpG islands. Sixty-two of these genes were weakly methylated in promoter regions. Among these, 48.4% were highly methylated in the gene body and 29.0% displayed weak gene-body methylation.

In summary, these experiments demonstrated that padlock probes can specifically extract a large number of genomic regions in single-tube reactions for bisulfite sequencing analysis. The degree of multiplexity is at least four orders of magnitude greater than that possible with conventional PCR-based bisulfite sequencing. The high capturing specificity is contributed by the cooperative annealing of the two capturing arms on the target molecules in proper orientation and distance, the selectivity of DNA polymerase and ligase, and the removal of linear DNA with exonuclease.

Although padlock probes have been successfully applied to exon capturing (Porreca, G. J. et al. (2007) Nat. Methods 4: 931-936) and SNP genotyping (Hardenbol, P. et al. (2003) Nat. Biotechnol. 21: 673-678), these experiments demonstrate their use with bisulfite-converted DNA with highly skewed nucleotide composition and low sequence complexity. Recent studies showed that most methylation changes are restricted to a very small fraction of the genome outside of CpG islands (Meissner, A. et al. (2008) Nature 454: 766-770; Ball, M. P. et al. (2009) Nat. Biotechnol. 27(4): 361-8; Irizarry, R. A. et al. (2009) Nat. Genet. 41: 178-186). Padlock capture is more efficient than full-genome bisulfite sequencing (Cokus, S. J. et al. (2008) Nature 452: 215-219; Lister, R. et al. (2008) Cell 133: 523-536) for quantifying DNA methylation, as it allows for focused sequencing on the most informative genomic regions. It also provides a much greater flexibility than reduced representation bisulfite sequencing (Meissner, A. et al. (2008) Nature 454: 766-770; Ball, M. P. et al. (2009) Nat. Biotechnol. 27(4): 361-8) in the selection of genomic targets, because the latter method is limited to genomic regions closely adjacent to the recognition sites of restriction enzymes.

### Example 2

### Library-Free Methylation Sequencing with Bisulfite Padlock Probes

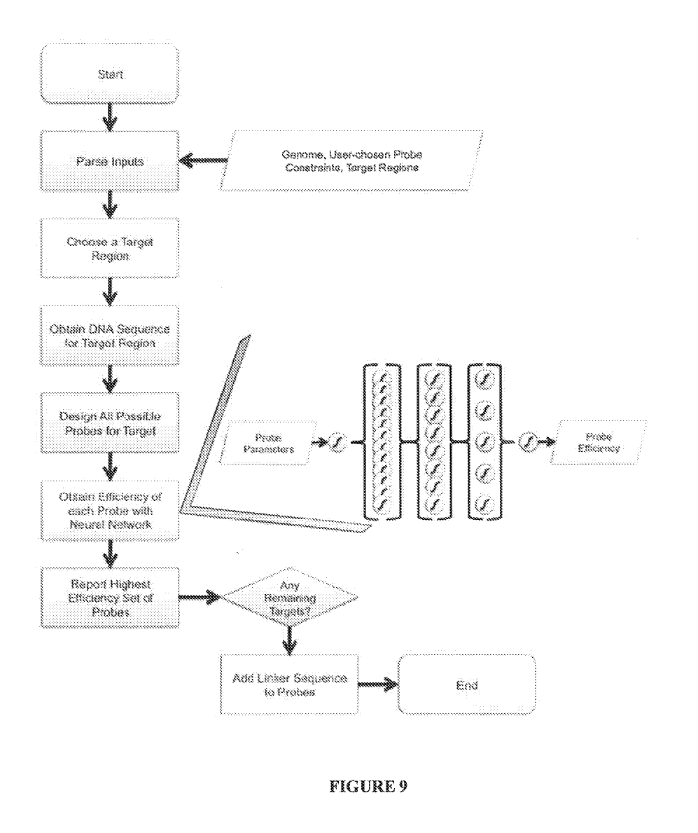

The program ppDesigner was developed to aid in the design of efficient padlock probes for bisulfite analysis. It accepts as input the genome of any organism, a list of user-specified arbitrary targets and user-desired probe constraints matching requirements of the experimental protocol. It ‘bisulfite-converts’ the genome in silico (that is, it changes all cytosines to thymines) and outputs padlock probes to cover the chosen targets while avoiding CpGs on the capturing arms that could be methylated and not converted to be recognized as thymine. ppDesigner uses a back-propagation neural network to predict probe efficiency (FIG. 9). This network was previously trained using data from probes for exomic targets (Gore, A. et al. (2011) Nature 471: 63-67) based on seven properties. Using bisulfite capture data from the first BSPPs (Deng, J. et al. (2009) Nat. Biotechnol. 27: 353-360), the network was refined with two additional factors. ppDesigner can explain ˜50% of the variance in capturing efficiency for genomic DNA and ˜20% of the variance in capturing efficiency for bisulfite-converted DNA; additional variation could be due to factors such as variability in oligonucleotide synthesis and sample DNA quality. ppDesigner is extremely flexible and has been used to design a variety of genomic and bisulfite probes for Homo sapiens (Liu, G. H. et al. (2011) Nature 472: 221-225; Liu, G. H. et al. (2011) Cell Stem Cell 8: 688-694), Mus musculus (Xu, Y. et al. (2011) Mol. Cell 42: 451-464) and Drosophila melanogaster (Wang, H. et al. (2010) Genome Res. 29: 981-988).

Key requirements for methylation analysis of large sample sizes include low cost, simple workflow and automation compatibility. As the cost of DNA sequencing has rapidly decreased, sample processing has become a bottleneck in terms of cost and throughput. A complicated workflow increases variability between samples and reduces power in large-scale studies. To address these issues, we extended a “library-free” protocol (Turner, E. H. et al. (2009) Nat. Methods 6: 315-316) to multiplexed BSPP capture. This method eliminates five steps from Illumina's library-construction protocol such that multiplexed libraries can be generated from DNA in only four steps (Table 4). Table 4 herein shows the number of enzymatic reactions, number of purifications, cost per sample, and mapping rates for first-generation padlock probes, second-generation library-free padlock probes, reduced representation bisulfite sequencing (RRBS), and whole genome bisulfite sequencing (WGBS).

TABLE 9
Comparison of bisulfite sequencing methods

| | Published | Library-Free | | |
|---|---|---|---|---|
| | BSPP | BSPP | RRBS | WGBS |
| Enzymatic reactions | 10 | 3 | 4 | 3 |
| Purification | 6 | 1 | 3 | 3 |
| Size-selection | 2 | 1¹ | 1 | 1 |
| Sample preparation | $71.15¹ | $37.86¹ | $28.15 | $31.10 |
| cost per sample | | | | |
| Mapping rate | 44% | 87% | 27%³ | N.D. |
| Genome coverage | <0.1% | 0.6%-1% | ~1%³ | 76-96%⁴ |
| obtained at 10x | | | | |
| depth | | | | |
| Sequencing (Gbps) | 0.5 | 4.0 | 1.4 | 70.0 |
| Sequencing cost per | $24.38 | $195.00 | $68.25 | $3412.50 |
| sample⁵ | | | | |

¹Unlike other methods, in the library-free BSPP protocol, size selection is typically performed on 48-96 pooled libraries.
²Includes the cost of ordering 400,000 synthesized probes from LC Sciences and reagents for preparing probes, bisulfite conversion, capture, and sequencing library preparation. Estimates assume that 10,000 samples will be processed.
³Estimated from: Gu et al., (2010) Nat. Methods 7(2): 133-136.
⁴Adapted from: Beck et al., (2010) Nat. Biotechnol. 28: 1026-1028.
⁵Assumes sequencing using an Illumina HiSeq to generate 300 Gbps of sequencing data, with cost of $4920 for a flowcell, $6815 for sequencing reagents, and $2890 for service fee. ($48.75 per Gbps)

Using multiplexed primers with 6-base pair (bp) barcodes, libraries for 96 samples in 96-well plates were routinely generated and sequenced all at once in a single Illumina HiSeq flowcell. Additionally, barcodes to process 384 samples per batch were designed. As sample-specific barcodes were added, barcoded libraries can be pooled for size selection, which is the most time consuming, contamination-prone and error-prone step if performed individually. The protocol is compatible with the use of multichannel pipettes or liquid-handling devices. It dramatically reduced experimental cost and time, and improved reproducibility and read mapping rates (Tables 4 and 5).

TABLE 5
Representative cost per sample for oligonucleotide synthesis,
sequencing library construction, and Illumina sequencing

| Expected number of | Probe Set Sizes | | |
|---|---|---|---|
| samples to be processed | 4,000 | 40,000 | 400,000 |
| 10 | $134.57 | $872.04 | $9,298.78 |
| 100 | $35.57 | $129.54 | $1,131.28 |
| 1000 | $25.67 | $55.29 | $314.53 |
| 10,000 | $24.68 | $47.86 | $232.86 |

For large sample sizes, the library preparation cost (including probes) with our protocol was comparable to that of the restricted-representation bisulfite sequencing and whole-genome bisulfite sequencing protocols, and the sequencing cost was much lower than that of whole-genome bisulfite sequencing owing to targeting of CpG sites of interest. Restricted-representation bisulfite sequencing is more cost-effective than BSPPs, but the former lacks BSPPs' flexibility in selecting specific sites or regions.

Another bottleneck in sequencing of bisulfite-converted DNA is a lack of computational tools to efficiently analyze sequencing data generated from hundreds of samples. To overcome this issue, an analysis pipeline for read mapping and methylation quantification was developed, called bisReadMapper. In previous padlock probe studies, reads had been mapped only against target regions owing to the computational requirements of sequence alignment (Deng, J. et al. (2009) Nat. Biotechnol. 27: 353-360). In contrast, bisReadMapper was designed to map to the full genome sequence, allowing processing of data from both targeted and whole-genome sequencing of bisulfite-converted DNA. bisReadMapper also determines the origin strand of the read based on base composition and maps reads as if they were fully bisulfite-converted to a fully bisulfite-converted genome sequence, allowing mapping of both bi- and unidirectional bisulfite libraries in an unbiased manner. Another feature is the capability to call single-nucleotide polymorphisms (SNPs) from sequences of bisulfite-converted DNA; this feature not only allows for analysis of allele-specific methylation (Shoemaker, R. et al. (2010) Genome Res. 20: 883-889) but also allows accurate sample tracking in large-scale experiments. Finally, bisReadMapper can call methylation levels at both CpG and non-CpG sites.

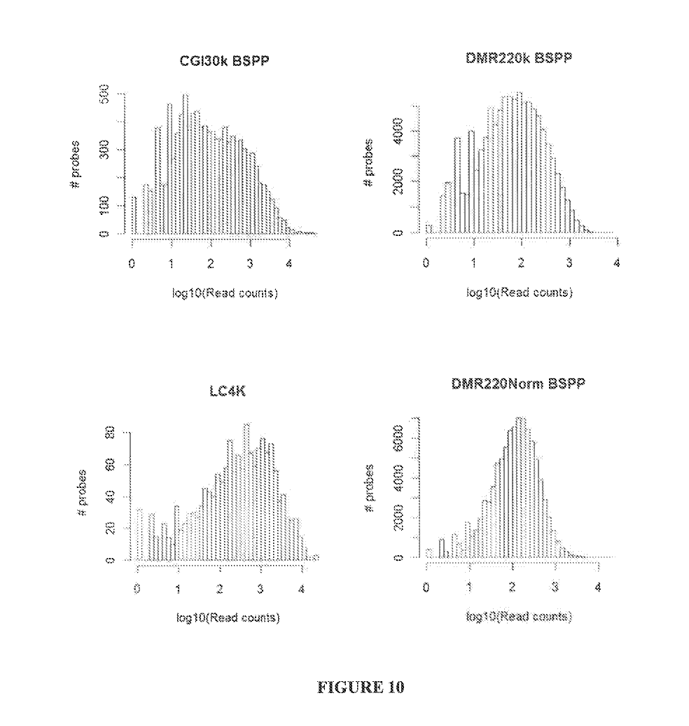

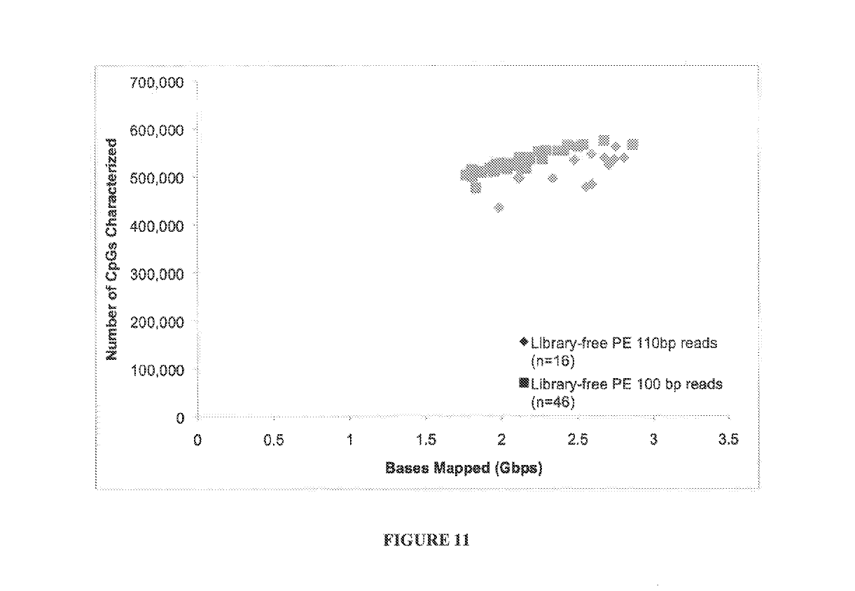

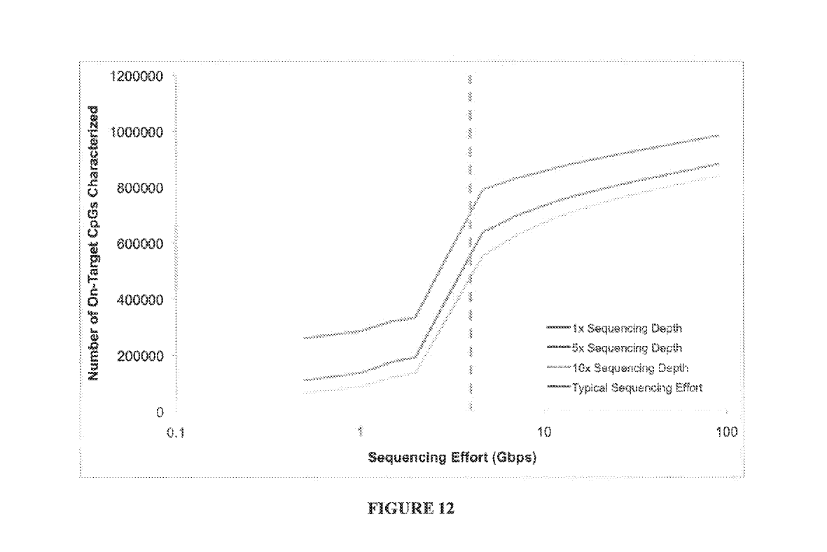

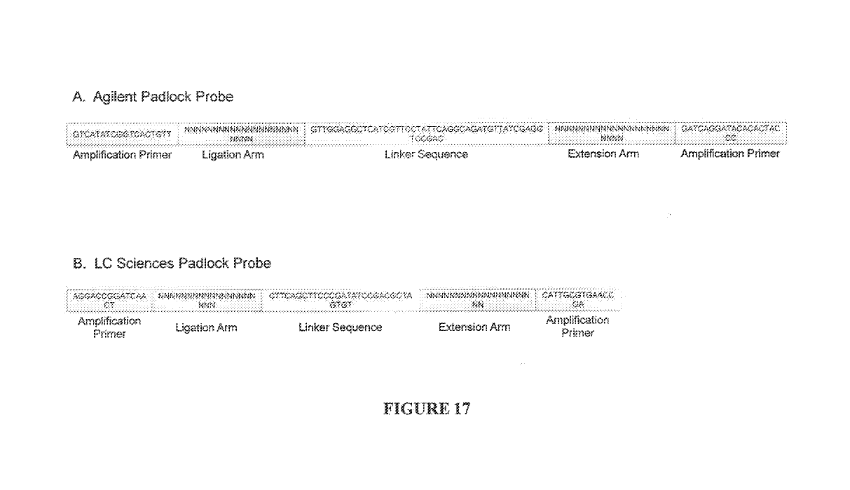

To test this assay, a genome-scale probe set based on our previous results was generated, as well as new information about differential methylation (Deng, J. et al. (2009) Nat. Biotechnol. 27: 353-360). The new design was targeted for evaluation of methylation at genomic locations known to contain differentially methylated regions or differentially methylated sites (DMSs; Irizarry, R. A. et al. (2009) Nat. Genet. 41: 178-186; Doi, A. et al. (2009) Nat. Genet. 41: 1350-1353; Lister, R. et al. (2009) Nature 462: 315-322; Figueroa, M. E. et al. (2010) Cancer Cell 17: 13-27), transcriptional repressor CTCF binding sites and DNase I hypersensitive regions. All microRNA genes and all promoters for human U.S. National Center for Biotechnology Information reference sequence (RefSeq) genes were targeted. Using ppDesigner, ˜330,000 padlock probes that covered 140,749 non-overlapping regions with a total size of 34 megabases were designed. Capturing experiments and end-sequencing were performed, and found that these probes were slightly more specific (˜96% on-target) and uniform than previous probes (Deng, J. et al. (2009) Nat. Biotechnol. 27: 353-360) (FIG. 10). To improve uniformity, the experimental capturing performance of these probes was normalized using subsetting and suppressor oligonucleotides as described previously (Deng, J. et al. (2009) Nat. Biotechnol. 27: 353-360). Roughly 500,000 CpG sites with ˜4 gigabases of sequencing reads could be characterized, and additional sites became callable with deeper sequencing (FIGS. 11 and 12). A schematic for the padlock probes is illustrated in FIG. 17.

### Materials and Methods

Bisulfite Padlock Probe Production (Oligonucleotides from Agilent)

Libraries of oligonucleotides (˜150 nt) were synthesized by ink-jet printing on programmable microarrays (Agilent Technologies) and released to form a combined library of 330,000 oligonucleotides. The oligonucleotides were amplified by PCR in 96 reactions (100 μl each) with 0.02 nM template oligonucleotide, 400 nM each of pAP1V61U primer and AP2V6 primer (Table 6), and 50 μl of KAPA SYBG fast Universal 2× qPCR Master Mix (Kapabiosystems) at 95° C. for 30 seconds, 15-16 cycles of 95° C. for 3 seconds; 55° C. for 30 seconds; and 60° C. for 20 seconds and 60° C. for 2 minutes.

TABLE 6
Primer sequences used for padlock probe production, padlock capture,
sequencing library construction, and Illumina sequencing

| | |
|---|---|
| Primer Name | Primer Sequences |

Primers used with Agilent Probes

| | |
|---|---|
| pAP1V61U | 5′-G*G*G*TCATATCGGTCACTGTU-3′ (SEQ ID NO: 8) |
| AP2V6 | 5′-/5Phos/CACGGGTAGTGTGTATCCTG-3′ (SEQ ID NO: 9) |
| RE-DpnII-V6 | 5′-GTGTATCCTGATC-3′ (SEQ ID NO: 10) |
| AmpF6.4Sol | 5′-AATGATACGGCGACCACCGAGATCTACACCACTCTCAGATGTTA |
| | TCGAGGTCCGAC-3′ (SEQ ID NO: 11) |
| AmpF6.3NH2 | 5′-/5AmMC6/CAGATGTTATCGAGGTCCGAC-3′ (SEQ ID NO: 12) |
| AmpR6.3NH2 | 5′-/5AmMC6/GGAACGATGAGCCTCCAAC-3′ (SEQ ID NO: 13) |
| PCR_F | 5′-AATGATACGGCGACCACCGAGATCTACACTCTTTCCCTACACGAC |
| | GCTCTTC-3′ (SEQ ID NO: 14) |
| PE_t_N2 | 5′-ACACTCTTTCCCTACACGACGCTCTTCCGATCTN*N-3′ |
| | (SEQ ID NO: 15) |
| PE_b_A | 5′/5Phos/AGATCGGAAGAGCGGTTCAGCAGGAATGCCGAG-3′ |
| | (SEQ ID NO: 16) |
| SolSeq6.3.3 (Read1) | 5′-TACACCACTCTCAGATGTTATCGAGGTCCGAC-3′ (SEQ ID NO: 17) |
| SolSeqV6.3.2r (Read2) | 5′-GCTAGGAACGATGAGCCTCCAAC-3′ (SEQ ID NO: 18) |
| AmpR6.3IndSeq (IndexRead) | 5′-GTTGGAGGCTCATCGTTCCTAGC-3′ (SEQ ID NO: 19) |

Primers used with LC Sciences Probes

| | |
|---|---|
| eMIP_CA1_F | 5′-TGCCTAGGACCGGATCAACT-3′ (SEQ ID NO: 20) |
| cMIP_CA1_R | 5′-GAGCTTCGUTTCACGCAATG-3′ (SEQ ID NO: 21) |
| CP-2-FA | 5′-GCACGATCCGACGGTAGTGT-3′ (SEQ ID NO: 22) |
| CP-2-RA | 5′-CCGTAATCGGGAAGCTGAAG-3′ (SEQ ID NO: 23) |
| CA-2-FA.Indx7Sol | 5′-CAAGCAGAAGACGGCATACGAGATGATCTGCGGTCTGCCATCCG |
| | ACGGTAGTGT-3′ (SEQ ID NO: 24) |
| CA-2-FA.Indx45Sol | 5′-CAAGCAGAAGACGGCATACGAGATCGTAGTCGGTCTGCCATCCG |
| | ACGGTAGTGT-3′ (SEQ ID NO: 25) |
| CA-2-FA.Indx76Sol | 5′-CAAGCAGAAGACGGCATACGAGATAATAGGCGGTCTGCCATCCG |
| | ACGGTAGTGT-3′ (SEQ ID NO: 26) |
| CA-2-RA.Sol | 5′-AATGATACGGCGACCACCGAGATCTACACGCCTATCGGGAAGCT |
| | GAAG-3′ (SEQ ID NO: 27) |
| Switch.CA-2-FA.Sol | 5′-AATGATACGGCGACCACCGAGATCTACACGCCTATCCGACGGTA |
| | GTGT-3′ (SEQ ID NO: 28) |
| Switch.CA-2-RA.Ind7Sol | 5′-CAAGCAGAAGACGGCATACGAGATGATCTGCGGTCTGCCATCGG |
| | GAAGCTGAAG-3′ (SEQ ID NO: 29) |
| Switch.CA-2-RA.Ind45Sol | 5′-CAAGCAGAAGACGGCATACGAGATCGTAGTCGGTCTGCCATCGG |
| | GAAGCTGAAG-3′ (SEQ ID NO: 30) |
| Switch.CA-2-RA.Ind76Sol | 5′-CAAGCAGAAGACGGCATACGAGATAATAGGCGGTCTGCCATCG |
| | GGAAGCTGAAG-3′ (SEQ ID NO: 31) |
| CP-2-SeqRead1.x (Read1) | 5′-TACACGCCTATCGGGAAGCTGAAG-3′ (SEQ ID NO: 32) |
| CP-2-IndSeq.x (IndexRead) | 5′-ACACTACCGTCGGATGGCAGACCG-3′ (SEQ ID NO: 33) |
| CP-2-SeqRead1.y (Read1) | 5′-TACACGCCTATCCGACGGTAGTGT-3′ (SEQ ID NO: 34) |
| CP-2-IndSeq.y (IndexRead) | 5′-CTTCAGCTTCCCGATGGCAGACCG-3′ (SEQ 1D NO: 35) |

*Indicates a phosphorothioate bond

The amplicons were purified by ethanol precipitation and repurified with Qiaquick PCR purification columns (Qiagen). Approximately 20 μg of the purified amplicons were digested with 50 units of lambda exonuclease (5 U/μl; New England Biolabs (NEB)) at 37° C. for 1 hour in lambda exonuclease reaction buffer. The resulting single-strand amplicons were purified with Qiaquick PCR purification column. Approximately 5-8 μg of single strand amplicons were subsequently digested with 5 units USER (1 U μ-1, NEB) at 37° C. for 1 hour. The digested DNA was annealed to 5.88 μM RE-DpnII-V6 guide oligo (Table 6) and denatured at 94° C. for 2 minutes decreased the temperature to 37° C. and incubated at 37° C. for 3 minutes. The mixture was digested with 50 U of DpnII (10 U μl-1, NEB) in NEBuffer DpnII at 37° C. for 2 hours. The mixture was further digested with 5 U of USER at 37° C. for 2 hours followed by enzyme inactivation at 75° C. for 20 minutes. The USER and DpnII-digested DNA was purified with Qiaquick PCR purification column. The single-strand 102-nt probes were purified with 6% denaturing PAGE (6% TB-urea two dimensional (2D) gel; Invitrogen).

Bisulfite Padlock Probe Production (Oligonucleotides from LC Sciences)

The oligonucleotides (100 nt) were synthesized using a programmable microfluidic microarray platform (LC Sciences) and released to form a mix of 3,918 oligonucleotides. The oligonucleotides were amplified by two-step PCR in a 200 μl reaction with 1 nM template oligonucleotides, 400 nM each of eMIP_CA1_F primer and eMIP_CA1_R primer (Table 6), and 100 μl of KAPA SYBR fast Universal qPCR Master Mix at 95° C. for 30 seconds, 5 cycles of 95° C. for 5 seconds; 52° C. for 1 minute; and 72° C. for 30 seconds, 10-12 cycles of 95° C. for 5 seconds; 60° C. for 30 seconds; and 72° C. for 30 seconds, and 72° C. for 2 minutes. The resultant amplicons were purified with Qiaquick PCR purification columns and re-amplified in 32 PCRs (100 μl each) with 0.02 nM first round amplicons, 400 nM each of eMIP_CA1_F primer and eMIP_CA1_R primer and 50 μl of KAPA SYBR fast Universal qPCR master mix at 95° C. for 30 seconds, 13-15 cycles of 95° C. for 5 seconds; 60° C. for 30 seconds; and 72° C. for 30 seconds and 72° C. for 2 minutes. The resultant amplicons were purified by ethanol precipitation and repurified with Qiaquick PCR purification columns as described above. Approximately 4 μg of the purified amplicons were digested with 100 U of Nt.AlwI (100 U/μl, NEB) at 37° C. for 1 hour in NEBuffer 2. The enzyme was heat-inactivated at 80° C. for 20 minutes. The digested amplicons were then incubated with 100 U of Nb.BrsDI (10 U μl-1, NEB) at 65° C. for 1 hour. The nicked DNA was purified by Qiaquick PCR purification column. The probe molecules (˜70 bases) were purified by 6% denaturing PAGE (6% TB-urea 2D gel).

Sample Preparation and Capture

Genomic DNA was extracted using the AllPrep DNA/RNA Mini kit (Qiagen) and bisulfite-converted with the EZ-96 DNA methylation Gold kit (Zymoresearch) in a 96-well plate. Normalized amount of padlock probes, 200 ng of bisulfite converted gDNA and 4.2 nM oligo suppressor were mixed in 25 μl 1× Ampligase buffer (Epicentre) in 96-well plate, denatured at 95° C. for 10 minutes, gradually lowered the temperature at 0.02° C./s to 55° C. in a thermocycler and hybridized at 55° C. for 20 hours. 2.5 μl of SLN (Stoffel fragment, ligase, nucleotides) mix (100 μM dNTP, 2 U Stoffel Fragment (ABI) and 0.5 U/μl Ampligase (Epicentre) in 1× Ampligase buffer) was added to the reaction for gap-filling. For circularization, the reactions were incubated at 55° C. for 20 hours, followed by enzyme inactivation at 94° C. for 2 minutes. To digest linear DNA after circularization, 2 μl of exonuclease mix (10 U/μl exonuclease 1 and 100 U/μl exonuclease III, USB) was added to the reactions, and the reactions were incubated at 37° C. for 2 hours and then inactivated at 94° C. for 2 minutes.

Capture-Circles Amplification (Library-Free BSPP Protocol, Agilent Oligonucleotides)

Ten microliters of circularized DNA was amplified and barcoded in 100-μl reactions with 400 nM each of AmpF6.3Sol primer (Table 6) and AmpR6.3 indexing primer (Table 6), 0.4×SYBR Green I (Invitrogen) and 50 μl Phusion High-Fidelity 2× master mix (NEB) at 98° C. for 30 seconds, 5 cycles of 98° C. for 10 seconds; 58° C. for 20 seconds; and 72° C. for 20 seconds, 9-12 cycles of 98° C. for 10 seconds; and 72° C. for 20 seconds and 72° C. for 3 minutes.

Capture-Circles Amplification (Library-Free BSPP Protocol, LC Sciences Oligonucleotides)

Ten microliters of circularized DNA was amplified in a 100-μl reaction with 200 nM each of CP-2-FA primer and CP-2-RA primer (Table 6) and 50 μl KAPA SYBR fast Universal qPCR Master Mix at 98° C. for 30 seconds, 5 cycles of 98° C. for 10 seconds; 52° C. for 30 seconds; and 72° C. for 30 seconds, 15 cycles of 98° C. for 10 seconds; 60° C. for 30 seconds; and 72° C. for 30 seconds and 72° C. for 3 minutes. The resultant amplicons with the corresponding expected size of ˜260 bp were purified by 6% PAGE (6% 5-well gel, Invitrogen) and resuspended in 12 μl of TE buffer. Thirty percent of the gel-purified amplicons were reamplified and barcoded in a 100-μl reaction with 200 nM each of two different sets of primers to enable single-end sequencing of both ends of the amplicons (CP-2-FA.IndSol primer and CP-2-RA.Sol primer or Switch. CP-2-FA and Switch.CP-2-RA.IndSol) and 50 μl KAPA SYBR fast Universal qPCR Master Mix at 98° C. for 30 seconds, 4 cycles of 98° C. for 10 seconds; 54° C. for 30 seconds; and 72° C. for 30 seconds and 72° C. for 3 minutes.

Primer Barcode Design for Multiplexing

An in-house Pert script was written to randomly generate 6-nt-long sequences. A sequence was kept if it did not have more than two matching positions with another accepted barcode and if it had 2-4 guanines or cytosines. The script reiterated until the desired number of barcodes have been obtained. A total of 384 primers were designed (Table 7).

TABLE 7
Sequences of multiplexed barcoded primers used in the library-free protocol.

| | |
|---|---|
| Barcode | SEQ ID |

| ID | Barcode | Primer | NO: |
|---|---|---|---|
| Ind1 | CGTGAT | CAAGCAGAAGACGGCATACGAGATCCTTGCGCTAGGAACGATGAGCCT | 36 |
| | | CCAAC | |
| Ind2 | ACATCG | CAAGCAGAAGACGGCATACGAGATGTTGCGGCTAGGAACGATGAGCCT | 37 |
| | | CCAAC | |
| Ind3 | GCCTAA | CAAGCAGAAGACGGCATACGAGATTCAGTTGCTAGGAACGATGAGCCT | 38 |
| | | CCAAC | |
| Ind4 | TGGTCA | CAAGCAGAAGACGGCATACGAGATTGGTCAGCTAGGAACGATGAGCCT | 39 |
| | | CCAAC | |
| Ind5 | CACTGT | CAAGCAGAAGACGGCATACGAGATCACTGTGCTAGGAACGATGAGCCT | 40 |
| | | CCAAC | |
| Ind6 | ATTGGC | CAAGCAGAAGACGGCATACGAGATATTGGCGCTAGGAACGATGAGCCT | 41 |
| | | CCAAC | |
| Ind7 | GATCTG | CAAGCAGAAGACGGCATACGAGATGATCTGGCTAGGAACGATGAGCCT | 42 |
| | | CCAAC | |
| Ind8 | TCAAGT | CAAGCAGAAGACGGCATACGAGATTCAAGTGCTAGGAACGATGAGCCT | 43 |
| | | CCAAC | |
| Ind9 | CTGATC | CAAGCAGAAGACGGCATACGAGATCTGATCGCTAGGAACGATGAGCCT | 44 |
| | | CCAAC | |
| Ind10 | AAGCTA | CAAGCAGAAGACGGCATACGAGATAAGCTAGCTAGGAACGATGAGCCT | 45 |
| | | CCAAC | |
| Ind11 | GTAGCC | CAAGCAGAAGACGGCATACGAGATGTAGCCGCTAGGAACGATGAGCCT | 46 |
| | | CCAAC | |
| Ind12 | TACAAG | CAAGCAGAAGACGGCATACGAGATTACAAGGCTAGGAACGATGAGCCT | 47 |
| | | CCAAC | |
| Ind13 | TCATGG | CAAGCAGAAGACGGCATACGAGATTCATGGGCTAGGAACGATGAGCCT | 48 |
| | | CCAAC | |
| Ind14 | TGTCTT | CAAGCAGAAGACGGCATACGAGATTGTCTTGCTAGGAACGATGAGCCT | 49 |
| | | CCAAC | |
| Ind15 | AGGAAG | CAAGCAGAAGACGGCATACGAGATAGGAAGGCTAGGAACGATGAGCCT | 50 |
| | | CCAAC | |
| Ind16 | AACCCC | CAAGCAGAAGACGGCATACGAGATAACCCCGCTAGGAACGATGAGCCT | 51 |
| | | CCAAC | |
| Ind17 | GATGAG | CAAGCAGAAGACGGCATACGAGATGATGAGGCTAGGAACGATGAGCCT | 52 |
| | | CCAAC | |
| Ind18 | TGAACT | CAAGCAGAAGACGGCATACGAGATTGAACTGCTAGGAACGATGAGCCT | 53 |
| | | CCAAC | |
| Ind19 | TGCGTC | CAAGCAGAAGACGGCATACGAGATTGCGTCGCTAGGAACGATGAGCCT | 54 |
| | | CCAAC | |
| Ind20 | GACAGG | CAAGCAGAAGACGGCATACGAGATGACAGGGCTAGGAACGATGAGCCT | 55 |
| | | CCAAC | |
| Ind21 | GGGTTG | CAAGCAGAAGACGGCATACGAGATGGGTTGGCTAGGAACGATGAGCCT | 56 |
| | | CCAAC | |
| Ind22 | TCCGAG | CAAGCAGAAGACGGCATACGAGATTCCGAGGCTAGGAACGATGAGCCT | 57 |
| | | CCAAC | |
| Ind23 | TTTCGA | CAAGCAGAAGACGGCATACGAGATTTTCGAGCTAGGAACGATGAGCCT | 58 |
| | | CCAAC | |
| Ind24 | GCGAAT | CAAGCAGAAGACGGCATACGAGATGCGAATGCTAGGAACGATGAGCCT | 59 |
| | | CCAAC | |
| Ind25 | GCAGTA | CAAGCAGAAGACGGCATACGAGATGCAGTAGCTAGGAACGATGAGCCT | 60 |
| | | CCAAC | |
| Ind26 | TCACGA | CAAGCAGAAGACGGCATACGAGATTCACGAGCTAGGAACGATGAGCCT | 61 |
| | | CCAAC | |
| Ind27 | CGCGTA | CAAGCAGAAGACGGCATACGAGATCGCGTAGCTAGGAACGATGAGCCT | 62 |
| | | CCAAC | |
| Ind28 | GCACCT | CAAGCAGAAGACGGCATACGAGATGCACCTGCTAGGAACGATGAGCCT | 63 |
| | | CCAAC | |
| Ind29 | GTTCGT | CAAGCAGAAGACGGCATACGAGATGTTCGTGCTAGGAACGATGAGCCT | 64 |
| | | CCAAC | |
| Ind30 | CACTAA | CAAGCAGAAGACGGCATACGAGATCACTAAGCTAGGAACGATGAGCCT | 65 |
| | | CCAAC | |
| Ind31 | GTGGTG | CAAGCAGAAGACGGCATACGAGATGTGGTGGCTAGGAACGATGAGCCT | 66 |
| | | CCAAC | |
| Ind32 | CCTTGC | CAAGCAGAAGACGGCATACGAGATCCTTGCGCTAGGAACGATGAGCCT | 67 |
| | | CCAAC | |
| Ind33 | GTTGCG | CAAGCAGAAGACGGCATACGAGATGTTGCGGCTAGGAACGATGAGCCT | 68 |
| | | CCAAC | |
| Ind34 | TCAGTT | CAAGCAGAAGACGGCATACGAGATTCAGTTGCTAGGAACGATGAGCCT | 69 |
| | | CCAAC | |
| Ind35 | CCCGAT | CAAGCAGAAGACGGCATACGAGATCCCGATGCTAGGAACGATGAGCCT | 70 |
| | | CCAAC | |
| Ind36 | TGCTTG | CAAGCAGAAGACGGCATACGAGATTGCTTGGCTAGGAACGATGAGCCT | 71 |
| | | CCAAC | |
| Ind37 | TGTAGC | CAAGCAGAAGACGGCATACGAGATTGTAGCGCTAGGAACGATGAGCCT | 72 |
| | | CCAAC | |
| Ind38 | GCGTGA | CAAGCAGAAGACGGCATACGAGATGCGTGAGCTAGGAACGATGAGCCT | 73 |
| | | CCAAC | |
| Ind39 | CTCTGG | CAAGCAGAAGACGGCATACGAGATCTCTGGGCTAGGAACGATGAGCCT | 74 |
| | | CCAAC | |
| Ind40 | CCGTCA | CAAGCAGAAGACGGCATACGAGATCCGTCAGCTAGGAACGATGAGCCT | 75 |
| | | CCAAC | |
| Ind41 | GTTCCC | CAAGCAGAAGACGGCATACGAGATGTTCCCGCTAGGAACGATGAGCCT | 76 |
| | | CCAAC | |
| Ind42 | CTTTTC | CAAGCAGAAGACGGCATACGAGATCTTTTCGCTAGGAACGATGAGCCT | 77 |
| | | CCAAC | |
| Ind43 | GGCACT | CAAGCAGAAGACGGCATACGAGATGGCACTGCTAGGAACGATGAGCCT | 78 |
| | | CCAAC | |
| Ind44 | GGATGC | CAAGCAGAAGACGGCATACGAGATGGATGCGCTAGGAACGATGAGCCT | 79 |
| | | CCAAC | |
| Ind45 | CGTAGT | CAAGCAGAAGACGGCATACGAGATCGTAGTGCTAGGAACGATGAGCCT | 80 |
| | | CCAAC | |
| Ind46 | GAAATG | CAAGCAGAAGACGGCATACGAGATGAAATGGCTAGGAACGATGAGCCT | 81 |
| | | CCAAC | |
| Ind47 | GGAGAG | CAAGCAGAAGACGGCATACGAGATGGAGAGGCTAGGAACGATGAGCCT | 82 |
| | | CCAAC | |
| Ind48 | TAACGT | CAAGCAGAAGACGGCATACGAGATTAACGTGCTAGGAACGATGAGCCT | 83 |
| | | CCAAC | |
| Ind49 | ACACAG | CAAGCAGAAGACGGCATACGAGATACACAGGCTAGGAACGATGAGCCT | 84 |
| | | CCAAC | |
| Ind50 | AAAGGT | CAAGCAGAAGACGGCATACGAGATAAAGGTGCTAGGAACGATGAGCCT | 85 |
| | | CCAAC | |
| Ind51 | GCGATA | CAAGCAGAAGACGGCATACGAGATGCGATAGCTAGGAACGATGAGCCT | 86 |
| | | CCAAC | |
| Ind52 | CGTGTC | CAAGCAGAAGACGGCATACGAGATCGTGTCGCTAGGAACGATGAGCCT | 87 |
| | | CCAAC | |
| Ind53 | CTAGAA | CAAGCAGAAGACGGCATACGAGATGIAGAAGCTAGGAACGATGAGCCT | 88 |
| | | CCAAC | |
| Ind54 | GGACGT | CAAGCAGAAGACGGCATACGAGATGGACGTGCTAGGAACGATGAGCCT | 89 |
| | | CCAAC | |
| Ind55 | AGTCGA | CAAGCAGAAGACGGCATACGAGATAGTCGAGCTAGGAACGATGAGCCT | 90 |
| | | CCAAC | |
| Ind56 | GTCTGA | CAAGCAGAAGACGGCATACGAGATGTCTGAGCTAGGAACGATGAGCCT | 91 |
| | | CCAAC | |
| Ind57 | GAAGGA | CAAGCAGAAGACGGCATACGAGATGAAGGAGCTAGGAACGATGAGCCT | 92 |
| | | CCAAC | |
| Ind58 | ATGCTG | CAAGCAGAAGACGGCATACGAGATATGCTGGCTAGGAACGATGAGCCT | 93 |
| | | CCAAC | |
| Ind59 | TCTATC | CAAGCAGAAGACGGCATACGAGATTCTATCGCTAGGAACGATGAGCCT | 94 |
| | | CCAAC | |
| Ind60 | ATCTGT | CAAGCAGAAGACGGCATACGAGATATCTGTGCTAGGAACGATGAGCCT | 95 |
| | | CCAAC | |
| Ind61 | ATAGAG | CAAGCAGAAGACGGCATACGAGATATAGAGGCTAGGAACGATGAGCCT | 96 |
| | | CCAAC | |
| Ind62 | GCTAAA | CAAGCAGAAGACGGCATACGAGATGCTAAAGCTAGGAACGATGAGCCT | 97 |
| | | CCAAC | |
| Ind63 | ACCAGG | CAAGCAGAAGACGGCATACGAGATACCAGGGCTAGGAACGATGAGCCT | 98 |
| | | CCAAC | |
| Ind64 | CCAACT | CAAGCAGAAGACGGCATACGAGATCCAACTGCTAGGAACGATGAGCCT | 99 |
| | | CCAAC | |
| Ind65 | AAGGAA | CAAGCAGAAGACGGCATACGAGATAAGGAAGCTAGGAACGATGAGCCT | 100 |
| | | CCAAC | |
| Ind66 | CCTCCA | CAAGCAGAAGACGGCATACGAGATCCTCCAGCTAGGAACGATGAGCCT | 101 |
| | | CCAAC | |
| Ind67 | CACGTC | CAAGCAGAAGACGGCATACGAGATCACGTCGCTAGGAACGATGAGCCT | 102 |
| | | CCAAC | |
| Ind68 | CATAAC | CAAGCAGAAGACGGCATACGAGATCATAACGCTAGGAACGATGAGCCT | 103 |
| | | CCAAC | |
| Ind69 | CCATAT | CAAGCAGAAGACGGCATACGAGATCCATATGCTAGGAACGATGAGCCT | 104 |
| | | CCAAC | |
| Ind70 | GAAGTC | CAAGCAGAAGACGGCATACGAGATGAAGTCGCTAGGAACGATGAGCCT | 105 |
| | | CCAAC | |
| Ind71 | CAAAGA | CAAGCAGAAGACGGCATACGAGATCAAAGAGCTAGGAACGATGAGCCT | 106 |
| | | CCAAC | |
| Ind72 | TGGCAG | CAAGCAGAAGACGGCATACGAGATTGGCAGGCTAGGAACGATGAGCCT | 107 |
| | | CCAAC | |
| Ind73 | GAGTCC | CAAGCAGAAGACGGCATACGAGATGAGTCCGCTAGGAACGATGAGCCT | 108 |
| | | CCAAC | |
| Ind74 | TCGCCA | CAAGCAGAAGACGGCATACGAGATTCGCCAGCTAGGAACGATGAGCCT | 109 |
| | | CCAAC | |
| Ind75 | AAGTCG | CAAGCAGAAGACGGCATACGAGATAAGTCGGCTAGGAACGATGAGCCT | 110 |
| | | CCAAC | |
| Ind76 | AATAGG | CAAGCAGAAGACGGCATACGAGATAATAGGGCTAGGAACGATGAGCCT | 111 |
| | | CCAAC | |
| Ind77 | ACCCGT | CAAGCAGAAGACGGCATACGAGATACCCGTGCTAGGAACGATGAGCCT | 112 |
| | | CCAAC | |
| Ind78 | AACACG | CAAGCAGAAGACGGCATACGAGATAACACGGCTAGGAACGATGAGCCT | 113 |
| | | CCAAC | |
| Ind79 | GCTTGG | CAAGCAGAAGACGGCATACGAGATGCTTGGGCTAGGAACGATGAGCCT | 114 |
| | | CCAAC | |
| Ind80 | TTACCA | CAAGCAGAAGACGGCATACGAGATTTACCAGCTAGGAACGATGAGCCT | 115 |
| | | CCAAC | |
| Ind81 | CCAGGT | CAAGCAGAAGACGGCATACGAGATCCAGGTGCTAGGAACGATGAGCCI | 116 |
| | | CCAAC | |
| Ind82 | CGTTTG | CAAGCAGAAGACGGCATACGAGATCGTTTGGCTAGGAACGATGAGCCT | 117 |
| | | CCAAC | |
| Ind83 | GACCAC | CAAGCAGAAGACGGCATACGAGATGACCACGCTAGGAACGATGAGCCT | 118 |
| | | CCAAC | |
| Ind84 | ACAAGA | CAAGCAGAAGACGGCATACGAGATACAAGAGCTAGGAACGATGAGCCT | 119 |
| | | CCAAC | |
| Ind85 | ACCGCA | CAAGCAGAAGACGGCATACGAGATACCGCAGCTAGGAACGATGAGCCT | 120 |
| | | CCAAC | |
| Ind86 | TGGGTA | CAAGCAGAAGACGGCATACGAGATTGGGTAGCTAGGAACGATGAGCCT | 121 |
| | | CCAAC | |
| Ind87 | ATTCCG | CAAGCAGAAGACGGCATACGAGATATTCCGGCTAGGAACGATGAGCCT | 122 |
| | | CCAAC | |
| Ind88 | GAATGT | CAAGCAGAAGACGGCATACGAGATGAATGTGCTAGGAACGATGAGCCT | 123 |
| | | CCAAC | |
| Ind89 | GCTGAT | CAAGCAGAAGACGGCATACGAGATGCTGATGCTAGGAACGATGAGCCT | 124 |
| | | CCAAC | |
| Ind90 | AGTGCT | CAAGCAGAAGACGGCATACGAGATAGTGCTGCTAGGAACGATGAGCCT | 125 |
| | | CCAAC | |
| Ind91 | CAGGGA | CAAGCAGAAGACGGCATACGAGATCAGGGAGCTAGGAACGATGAGCCT | 126 |
| | | CCAAC | |
| Ind92 | CATGCG | CAAGCAGAAGACGGCATACGAGATCATGCGGCTAGGAACGATGAGCCT | 127 |
| | | CCAAC | |
| Ind93 | TGCCTA | CAAGCAGAAGACGGCATACGAGATTGCCTAGCTAGGAACGATGAGCCT | 128 |
| | | CCAAC | |
| Ind94 | CTATAC | CAAGCAGAAGACGGCATACGAGATCTATACGCTAGGAACGATGAGCCT | 129 |
| | | CCAAC | |
| Ind95 | CCGAGT | CAAGCAGAAGACGGCATACGAGATCCGAGTGCTAGGAACGATGAGCCT | 130 |
| | | CCAAC | |
| Ind96 | ACCTGC | CAAGCAGAAGACGGCATACGAGATACCTGCGCTAGGAACGATGAGCCT | 131 |
| | | CCAAC | |
| Ind97 | CAGGAC | CAAGCAGAAGACGGCATACGAGATCAGGACGCTAGGAACGATGAGCCT | 132 |
| | | CCAAC | |
| Ind98 | AAGATG | CAAGCAGAAGACGGCATACGAGATAAGATGGCTAGGAACGATGAGCCT | 133 |
| | | CCAAC | |
| Ind99 | GGCTTC | CAAGCAGAAGACGGCATACGAGATGGCTTCGCTAGGAACGATGAGCCT | 134 |
| | | CCAAC | |
| Ind100 | GTGCTT | CAAGCAGAAGACGGCATACGAGATGTGCTTGCTAGGAACGATGAGCCT | 135 |
| | | CCAAC | |
| Ind101 | TGCGCA | CAAGCAGAAGACGGCATACGAGATTGCGCAGCTAGGAACGATGAGCCT | 136 |
| | | CCAAC | |
| Ind102 | ACTAGC | CAAGCAGAAGACGGCATACGAGATACTAGCGCTAGGAACGATGAGCCT | 137 |
| | | CCAAC | |
| Ind103 | TCGAAG | CAAGCAGAAGACGGCATACGAGATTCGAAGGCTAGGAACGATGAGCCT | 138 |
| | | CCAAC | |
| Ind104 | AGACTA | CAAGCAGAAGACGGCATACGAGATAGACTAGCTAGGAACGATGAGCCT | 139 |
| | | CCAAC | |
| Ind105 | CGGGTT | CAAGCAGAAGACGGCATACGAGATCGGGTTGCTAGGAACGATGAGCCT | 140 |
| | | CCAAC | |
| Ind106 | TGACTG | CAAGCAGAAGACGGCATACGAGATTGACTGGCTAGGAACGATGAGCCT | 141 |
| | | CCAAC | |
| Ind107 | TTGTGT | CAAGCAGAAGACGGCATACGAGATTTGTGTGCTAGGAACGATGAGCCT | 142 |
| | | CCAAC | |
| Ind108 | TCGCTG | CAAGCAGAAGACGGCATACGAGATTCGCTGGCTAGGAACGATGAGCCT | 143 |
| | | CCAAC | |
| Ind109 | GATACA | CAAGCAGAAGACGGCATACGAGATGATACAGCTAGGAACGATGAGCCT | 144 |
| | | CCAAC | |
| Ind110 | TCCTTA | CAAGCAGAAGACGGCATACGAGATTCCTTAGCTAGGAACGATGAGCCT | 145 |
| | | CCAAC | |
| Ind111 | CGATTT | CAAGCAGAAGACGGCATACGAGATCGATTTGCTAGGAACGATGAGCCT | 146 |
| | | CCAAC | |
| Ind112 | TTACGG | CAAGCAGAAGACGGCATACGAGATTTACGGGCTAGGAACGATGAGCCT | 147 |
| | | CCAAC | |
| Ind113 | TGTGAC | CAAGCAGAAGACGGCATACGAGATTGTGACGCTAGGAACGATGAGCCT | 148 |
| | | CCAAC | |
| Ind114 | TTGGAA | CAAGCAGAAGACGGCATACGAGATTTGGAAGCTAGGAACGATGAGCCT | 149 |
| | | CCAAC | |
| Ind115 | ACCATA | CAAGCAGAAGACGGCATACGAGATACCATAGCTAGGAACGATGAGCCT | 150 |
| | | CCAAC | |
| Ind116 | GTCGAG | CAAGCAGAAGACGGCATACGAGATGTCGAGGCTAGGAACGATGAGCCT | 151 |
| | | CCAAC | |
| Ind117 | ACTGCC | CAAGCAGAAGACGGCATACGAGATACTGCCGCTAGGAACGATGAGCCT | 152 |
| | | CCAAC | |
| Ind118 | TCGGGT | CAAGCAGAAGACGGCATACGAGATTCGGGTGCTAGGAACGATGAGCCT | 153 |
| | | CCAAC | |
| Ind119 | GGCATG | CAAGCAGAAGACGGCATACGAGATGGCATGGCTAGGAACGATGAGCCT | 154 |
| | | CCAAC | |
| Ind120 | GTTTCT | CAAGCAGAAGACGGCATACGAGATGTTTCTGCTAGGAACGATGAGCCT | 155 |
| | | CCAAC | |
| Ind121 | ACCAAT | CAAGCAGAAGACGGCATACGAGATACCAATGCTAGGAACGATGAGCCT | 156 |
| | | CCAAC | |
| Ind122 | ATGCAT | CAAGCAGAAGACGGCATACGAGATATGCATGCTAGGAACGATGAGCCT | 157 |
| | | CCAAC | |
| Ind123 | TACGGC | CAAGCAGAAGACGGCATACGAGATTACGGCGCTAGGAACGATGAGCCT | 158 |
| | | CCAAC | |
| Ind124 | AGTCCC | CAAGCAGAAGACGGCATACGAGATAGTCCCGCTAGGAACGATGAGCCT | 159 |
| | | CCAAC | |
| Ind125 | CTGCAG | CAAGCAGAAGACGGCATACGAGATCTGCAGGCTAGGAACGATGAGCCT | 160 |
| | | CCAAC | |
| Ind126 | CTGTTG | CAAGCAGAAGACGGCATACGAGATCTGTTGGCTAGGAACGATGAGCCT | 161 |
| | | CCAAC | |
| Ind127 | CGGACA | CAAGCAGAAGACGGCATACGAGATCGGACAGCTAGGAACGATGAGCCT | 162 |
| | | CCAAC | |
| Ind128 | TAAGCG | CAAGCAGAAGACGGCATACGAGATTAAGCGGCTAGGAACGATGAGCCT | 163 |
| | | CCAAC | |
| Ind129 | GAGAGT | CAAGCAGAAGACGGCATACGAGATGAGAGTGCTAGGAACGATGAGCCT | 164 |
| | | CCAAC | |
| Ind130 | TACCCG | CAAGCAGAAGACGGCATACGAGATTACCCGCCTAGGAACGATGAGCCT | 165 |
| | | CCAAC | |
| Ind131 | TTCGTA | CAAGCAGAAGACGGCATACGAGATTTCGTAGCTAGGAACGATGAGCCT | 166 |
| | | CCAAC | |
| Ind132 | AAAGTG | CAAGCAGAAGACGGCATACGAGATAAAGTGGCTAGGAACGATGAGCCT | 167 |
| | | CCAAC | |
| Ind133 | TTTGGT | CAAGCAGAAGACGGCATACGAGATTTTGGTGCTAGGAACGATGAGCCT | 168 |
| | | CCAAC | |
| Ind134 | GTCCCT | CAAGCAGAAGACGGCATACGAGATGTCCCTGCTAGGAACGATGAGCCT | 169 |
| | | CCAAC | |
| Ind135 | TAGCTT | CAACCAGAAdAbGGCATACGAGATTAGCTIGCTAGGAACGATGAGCCT | 170 |
| | | CCAAC | |
| Ind136 | GCACTG | CAAGCAGAAGACGGCATACGAGATGCACTGGCTAGCAACGATGAGCCT | 171 |
| | | CCAAC | |
| Ind137 | ACTATG | CAAGCAGAAGACGGCATACGAGATACTATGGCTAGGAACGATGAGCCT | 172 |
| | | CCAAC | |
| Ind138 | GAACTT | CAAGCAGAAGACGGCATACGAGATGAACTTGCTAGGAACGATGAGCCT | 173 |
| | | CCAAC | |
| Ind139 | TTTGAG | CAAGCAGAAGACGGCATACGAGATTTTGAGGCTAGGAACGATGAGCCT | 174 |
| | | CCAAC | |
| Ind140 | AGGCCT | CAAGCAGAAGACGGCATACGAGATAGGCCTGCTAGGAACGATGAGCCT | 175 |
| | | CCAAC | |
| Ind141 | ACACGC | CAAGCAGAAGACGGCATACGAGATACACGCGCTAGGAACGATGAGCCT | 176 |
| | | CCAAC | |
| Ind142 | TTGTCG | CAAGCAGAAGACGGCATACGAGATTTGTCGGCTAGGAACGATGAGCCT | 177 |
| | | CCAAC | |
| Ind143 | TCTCAC | CAAGCAGAAGACGGCATACGAGATTCTCACGCTAGGAACGATGAGCCT | 178 |
| | | CCAAC | |
| Ind144 | TAGACC | CAAGCAGAAGACGGCATACGAGATTAGACCGCTAGGAACGATGAGCCT | 179 |
| | | CCAAC | |
| Ind145 | CCTAAG | CAAGCAGAAGACGGCATACGAGATCCTAAGGCTAGGAACGATGAGCCT | 180 |
| | | CCAAC | |
| Ind146 | GTATCG | CAAGCAGAAGACGGCATACGAGATGTATCGGCTAGGAACGATGAGCCT | 181 |
| | | CCAAC | |
| Ind147 | TCCAGA | CAAGCAGAAGACGGCATACGAGATTCCAGAGCTAGGAACGATGAGCCT | 182 |
| | | CCAAC | |
| Ind148 | AGGTGA | CAAGCAGAAGACGGCATACGAGATAGGTGAGCTAGGAACGATGAGCCT | 183 |
| | | CCAAC | |
| Ind149 | CCCATC | CAAGCAGAAGACGGCATACGAGATCCCATCGCTAGGAACGATGAGCCT | 184 |
| | | CCAAC | |
| Ind150 | TTGCAC | CAAGCAGAAGACGGCATACGAGATTTGCACGCTAGGAACGATGAGCCT | 185 |
| | | CCAAC | |
| Ind151 | AACTCA | CAAGCAGAAGACGGCATACGAGATAACTCAGCTAGGAACGATGAGCCT | 186 |
| | | CCAAC | |
| Ind152 | CGTATA | CAAGCAGAAGACGGCATACGAGATCGTATAGCTAGGAACGATGAGCCT | 187 |
| | | CCAAC | |
| Ind153 | AGCGAA | CAAGCAGAAGACGGCATACGAGATAGCGAAGCTAGGAACGATGAGCCT | 188 |
| | | CCAAC | |
| Ind154 | ACGGCT | CAAGCAGAAGACGGCATACGAGATACGGCTGCTAGGAACGATGAGCCT | 189 |
| | | CCAAC | |
| Ind155 | AGTGAG | CAAGCAGAAGACGGCATACGAGATAGTGAGGCTAGGAACGATGAGCCT | 190 |
| | | CCAAC | |
| Ind156 | TTTCTC | CAAGCAGAAGACGGCATACGAGATTTTCTCGCTAGGAACGATGAGCCT | 191 |
| | | CCAAC | |
| Ind157 | GCTCTA | CAAGCAGAAGACGGCATACGAGATGCTCTAGCTAGGAACGATGAGCCT | 192 |
| | | CCAAC | |
| Ind158 | ACTTGA | CAAGCAGAAGACGGCATACGAGATACTTGAGCTAGGAACGATGAGCCT | 193 |
| | | CCAAC | |
| Ind159 | CGGTTC | CAAGCAGAAGACGGCATACGAGATCGUTTCGCTAGGAACGATGAGCCT | 194 |
| | | CCAAC | |
| Ind160 | CATCAT | CAAGCAGAAGACGGCATACGAGATCATCATGCTAGGAACGATGAGCCT | 195 |
| | | CCAAC | |
| Ind161 | CAAACG | CAAGCAGAAGACGGCATACGAGATCAAACGGCTAGGAACGATGAGCCT | 196 |
| | | CCAAC | |
| Ind162 | CTATGT | CAAGCAGAAGACGGCATACGAGATCTATGTGCTAGGAACGATGAGCCT | 197 |
| | | CCAAC | |
| Ind163 | AGCGTT | CAAGCAGAAGACGGCATACGAGATAGCGTTGCTAGGAACGATGAGCCT | 198 |
| | | CCAAC | |
| Ind164 | AAGACT | CAAGCAGAAGACGGCATACGAGATAAGACTGCTAGGAACGATGAGCCT | 199 |
| | | CCAAC | |
| Ind165 | CGATAA | CAAGCAGAAGACGGCATACGAGATCGATAAGCTAGGAACGATGAGCCT | 200 |
| | | CCAAC | |
| Ind166 | CGGCTA | CAAGCAGAAGACGGCATACGAGATCGGCTAGCTAGGAACGATGAGCCT | 201 |
| | | CCAAC | |
| Ind167 | TATACG | CAAGCAGAAGACGGCATACGAGATTATACGGCTAGGAACGATGAGCCT | 202 |
| | | CCAAC | |
| Ind168 | GAAACC | CAAGCAGAAGACGGCATACGAGATGAAACCGCTAGGAACGATGAGCCT | 203 |
| | | CCAAC | |
| Ind169 | GAACCG | CAAGCAGAAGACGGCATACGAGATGAACCGGCTAGGAACGATGAGCCT | 204 |
| | | CCAAC | |
| Ind170 | TTAGGC | CAAGCAGAAGACGGCATACGAGATTTAGGCGCTAGGAACGATGAGCCT | 205 |
| | | CCAAC | |
| Ind171 | GCCTTT | CAAGCAGAAGACGGCATACGAGATGCCTTTGCTAGGAACGATGAGCCT | 206 |
| | | CCAAC | |
| Ind172 | GTTGGA | CAAGCAGAAGACGGCATACGAGATGTTGGAGCTAGGAACGATGAGCCT | 207 |
| | | CCAAC | |
| Ind173 | GTACGC | CAAGCAGAAGACGGCATACGAGATGTACGCGCTAGGAACGATGAGCCT | 208 |
| | | CCAAC | |
| Ind174 | TGGAAC | CAAGCAGAAGACGGCATACGAGATTGGAACGCTAGGAACGATGAGCCT | 209 |
| | | CCAAC | |
| Ind175 | AACAGA | CAAGCAGAAGACGGCATACGAGATAACAGAGCTAGGAACGATGAGCCT | 210 |
| | | CCAAC | |
| Ind176 | AAGCAC | CAAGCAGAAGACGGCATACGAGATAAGCACGCTAGGAACGATGAGCCT | 211 |
| | | CCAAC | |
| Ind177 | ATGGTA | CAAGCAGAAGACGGCATACGAGATATGGTAGCTAGGAACGATGAGCCT | 212 |
| | | CCAAC | |
| Ind178 | TCGTTC | CAAGCAGAAGACGGCATACGAGATTCGTTCGCTAGGAACGATGAGCCT | 213 |
| | | CCAAC | |
| Ind179 | CTTCTA | CAAGCAGAAGACGGCATACGAGATCTTCTAGCTAGGAACGATGAGCCT | 214 |
| | | CCAAC | |
| Ind180 | TGGGAT | CAAGCAGAAGACGGCATACGAGATTGGGATGCTAGGAACGATGAGCCT | 215 |
| | | CCAAC | |
| Ind181 | ATCGCC | CAAGCAGAAGACGGCATACGAGATATCGCCGCTAGGAACGATGAGCCT | 216 |
| | | CCAAC | |
| Ind182 | AGTTGG | CAAGCAGAAGACGGCATACGAGATAGTTGGGCTAGGAACGATGAGCCT | 217 |
| | | CCAAC | |
| Ind183 | AGCTCT | CAAGCAGAAGACGGCATACGAGATAGCTCTGCTAGGAACGATGAGCCT | 218 |
| | | CCAAC | |
| Ind184 | GACGGT | CAAGCAGAAGACGGCATACGAGATGACGGTGCTAGGAACGATGAGCCT | 219 |
| | | CCAAC | |
| Ind185 | CTCAGC | CAAGCAGAAGACGGCATACGAGATCTCAGCGCTAGGAACGATGAGCCT | 220 |
| | | CCAAC | |
| Ind186 | CTTAGG | CAAGCAGAAGACGGCATACGAGATCTTAGGGCTAGGAACGATGAGCCT | 221 |
| | | CCAAC | |
| Ind187 | CGACAG | CAAGCAGAAGACGGCATACGAGATCGACAGGCTAGGAACGATGAGCCT | 222 |
| | | CCAAC | |
| Ind188 | ACATGT | CAAGCAGAAGACGGCATACGAGATACATGTGCTAGGAACGATGAGCCT | 223 |
| | | CCAAC | |
| Ind189 | ATACGA | CAAGCAGAAGACGGCATACGAGATATACGAGCTAGGAACGATGAGCCT | 224 |
| | | CCAAC | |
| Ind190 | GAGCAT | CAAGCAGAAGACGGCATACGAGATGAGCATGCTAGGAACGATGAGCCT | 225 |
| | | CCAAC | |
| Ind191 | ATCCTA | CAAGCAGAAGACGGCATACGAGATATCCTAGCTAGGAACGATGAGCCT | 226 |
| | | CCAAC | |
| Ind192 | ACGTAA | CAAGCAGAAGACGGCATACGAGATACGTAAGCTAGGAACGATGAGCCT | 227 |
| | | CCAAC | |
| Ind193 | GGAAAC | CAAGCAGAAGACGGCATACGAGATGGAAACGCTAGGAACGATGAGCCT | 228 |
| | | CCAAC | |
| Ind194 | AGCAAC | CAAGCAGAAGACGGCATACGAGATAGCAACGCTAGGAACGATGAGCCT | 229 |
| | | CCAAC | |
| Ind195 | CTAGTG | CAAGCAGAAGACGGCATACGAGATCTAGTGGCTAGGAACGATGAGCCT | 230 |
| | | CCAAC | |
| Ind196 | CCGCTT | CAAGCAGAAGACGGCATACGAGATCCGCTTGCTAGGAACGATGAGCCT | 231 |
| | | CCAAC | |
| Ind197 | CCAGCA | CAAGCAGAAGACGGCATACGAGATCCAGCAGCTAGGAACGATGAGCCT | 232 |
| | | CCAAC | |
| Ind198 | ATACTC | CAAGCAGAAGACGGCATACGAGATATACTCGCTAGGAACGATGAGCCT | 233 |
| | | CCAAC | |
| Ind199 | GGTGAA | CAAGCAGAAGACGGCATACGAGATGGTGAAGCTAGGAACGATGAGCCT | 234 |
| | | CCAAC | |
| Ind200 | CTCTAT | CAAGCAGAAGACGGCATACGAGATCTCTATGCTAGGAACGATGAGCCT | 235 |
| | | CCAAC | |
| Ind201 | GACATA | CAAGCAGAAGACGGCATACGAGATGACATAGCTAGGAACGATGAGCCT | 236 |
| | | CCAAC | |
| Ind202 | GGATCT | CAAGCAGAAGACGGCATACGAGATGGATCTGCTAGGAACGATGAGCCT | 237 |
| | | CCAAC | |
| Ind203 | AACGAG | CAAGCAGAAGACGGCATACGAGATAACGAGGCTAGGAACGATGAGCCT | 238 |
| | | CCAAC | |
| Ind204 | CACCTG | CAAGCAGAAGACGGCATACGAGATCACCTGGCTAGGAACGATGAGCCT | 239 |
| | | CCAAC | |
| Ind205 | CATGAA | CAAGCAGAAGACGGCATACGAGATCATGAAGCTAGGAACGATGAGCCT | 240 |
| | | CCAAC | |
| Ind206 | CGTGGA | CAAGCAGAAGACGGCATACGAGATCGTGGAGCTAGGAACGATGAGCCT | 241 |
| | | CCAAC | |
| Ind207 | AGAAGG | CAAGCAGAAGACGGCATACGAGATAGAAGGGCTAGGAACGATGAGCCT | 242 |
| | | CCAAC | |
| Ind208 | ATCCAG | CAAGCAGAAGACGGCATACGAGATATCCAGGCTAGGAACGATGAGCCT | 243 |
| | | CCAAC | |
| Ind209 | TTCCTG | CAAGCAGAAGACGGCATACGAGATTTCCTGGCTAGGAACGATGAGCCT | 244 |
| | | CCAAC | |
| Ind210 | CAACAA | CAAGCAGAAGACGGCATACGAGATCAACAAGCTAGGAACGATGAGCCT | 245 |
| | | CCAAC | |
| Ind211 | CCTGTT | CAAGCAGAAGACGGCATACGAGATCCTGTTGCTAGGAACGATGAGCCT | 246 |
| | | CCAAC | |
| Ind212 | CTCGTT | CAAGCAGAAGACGGCATACGAGATCTCGTTGCTAGGAACGATGAGCCT | 247 |
| | | CCAAC | |
| Ind213 | CTGAGA | CAAGCAGAAGACGGCATACGAGATCTGAGAGCTAGGAACGATGAGCCT | 248 |
| | | CCAAC | |
| Ind214 | CGCTCA | CAAGCAGAAGACGGCATACGAGATCGCTCAGCTAGGAACGATGAGCCT | 249 |
| | | CCAAC | |
| Ind215 | TACCAA | CAAGCAGAAGACGGCATACGAGATTACCAAGCTAGGAACGATGAGCCT | 250 |
| | | CCAAC | |
| Ind216 | CGCAAG | CAAGCAGAAGACGGCATACGAGATCGCAAGGCTAGGAACGATGAGCCT | 251 |
| | | CCAAC | |
| Ind217 | TACTGA | CAAGCAGAAGACGGCATACGAGATTACTGAGCTAGGAACGATGAGCCT | 252 |
| | | CCAAC | |
| Ind218 | TCCACT | CAAGCAGAAGACGGCATACGAGATTCCACTGCTAGGAACGATGAGCCT | 253 |
| | | CCAAC | |
| Ind219 | ATCGTG | CAAGCAGAAGACGGCATACGAGATATCGTGGCTAGGAACGATGAGCCT | 254 |
| | | CCAAC | |
| Ind220 | TAGGCA | CAAGCAGAAGACGGCATACGAGATTAGGCAGCTAGGAACGATGAGCCT | 255 |
| | | CCAAC | |
| Ind221 | CAAGAG | CAAGCAGAAGACGGCATACGAGATCAAGAGGCTAGGAACGATGAGCCT | 256 |
| | | CCAAC | |
| Ind222 | ACGACA | CAAGCAGAAGACGGCATACGAGATACGACAGCTAGGAACGATGAGCCT | 257 |
| | | CCAAC | |
| Ind223 | GGTTCC | CAAGCAGAAGACGGCATACGAGATGGTTCCGCTAGGAACGATGAGCCT | 258 |
| | | CCAAC | |
| Ind224 | TCTTAG | CAAGCAGAAGACGGCATACGAGATTCTTAGGCTAGGAACGATGAGCCT | 259 |
| | | CCAAC | |
| Ind225 | AGAGGA | CAAGCAGAAGACGGCATACGAGATAGAGGAGCTAGGAACGATGAGCCT | 260 |
| | | CCAAC | |
| Ind226 | AAAGCA | CAAGCAGAAGACGGCATACGAGATAAAGCAGCTAGGAACGATGAGCCT | 261 |
| | | CCAAC | |
| Ind227 | AGTCAT | CAAGCAGAAGACGGCATACGAGATAGTCATGCTAGGAACGATGAGCCT | 262 |
| | | CCAAC | |
| Ind228 | TGCAGT | CAAGCAGAAGACGGCATACGAGATTGCAGTGCTAGGAACGATGAGCCT | 263 |
| | | CCAAC | |
| Ind229 | TGTTCT | CAAGCAGAAGACGGCATACGAGATTGTTCTGCTAGGAACGATGAGCCT | 264 |
| | | CCAAC | |
| Ind230 | TATCGC | CAAGCAGAAGACGGCATACGAGATTATCGCGCTAGGAACGATGAGCCT | 265 |
| | | CCAAC | |
| Ind231 | GCATCA | CAAGCAGAAGACGGCATACGAGATGCATCAGCTAGGAACGATGAGCCT | 266 |
| | | CCAAC | |
| Ind232 | CTCCGT | CAAGCAGAAGACGGCATACGAGATCTCCGTGCTAGGAACGATGAGCCT | 267 |
| | | CCAAC | |
| Ind233 | TAAGAC | CAAGCAGAAGACGGCATACGAGATTAAGACGCTAGGAACGATGAGCCT | 268 |
| | | CCAAC | |
| Ind234 | GAGGCT | CAAGCAGAAGACGGCATACGAGATGAGGCTGCTAGGAACGATGAGCCT | 269 |
| | | CCAAC | |
| Ind235 | TGATTC | CAAGCAGAAGACGGCATACGAGATTGATTCGCTAGGAACGATGAGCCT | 270 |
| | | CCAAC | |
| Ind236 | GTCCAA | CAAGCAGAAGACGGCATACGAGATGTCCAAGCTAGGAACGATGAGCCT | 271 |
| | | CCAAC | |
| Ind237 | ACTCTC | CAAGCAGAAGACGGCATACGAGATACTCTCGCTAGGAACGATGAGCCT | 272 |
| | | CCAAC | |
| Ind238 | TCAACG | CAAGCAGAAGACGGCATACGAGATTCAACGGCTAGGAACGATGAGCCT | 273 |
| | | CCAAC | |
| Ind239 | GGGTAC | CAAGCAGAAGACGGCATACGAGATGGGTACGCTAGGAACGATGAGCCT | 274 |
| | | CCAAC | |
| Ind240 | ACGATT | CAAGCAGAAGACGGCATACGAGATACGATTGCTAGGAACGATGAGCCT | 275 |
| | | CCAAC | |
| Ind241 | AACTTG | CAAGCAGAAGACGGCATACGAGATAACTTGGCTAGGAACGATGAGCCT | 276 |
| | | CCAAC | |
| Ind242 | AACGTA | CAAGCAGAAGACGGCATACGAGATAACGTAGCTAGGAACGATGAGCCT | 277 |
| | | CCAAC | |
| Ind243 | ACCCAC | CAAGCAGAAGACGGCATACGAGATACCCACGCTAGGAACGATGAGCCT | 278 |
| | | CCAAC | |
| Ind244 | CGGTCT | CAAGCAGAAGACGGCATACGAGATCGGTCTGCTAGGAACGATGAGCCT | 279 |
| | | CCAAC | |
| Ind245 | TTCTAG | CAAGCAGAAGACGGCATACGAGATTTCTAGGCTAGCAACGATGAGCCT | 280 |
| | | CCAAC | |
| Ind246 | GGTGTG | CAAGCAGAAGACGGCATACGAGATGGTGTGGCTAGGAACGATGAGCCT | 281 |
| | | CCAAC | |
| Ind247 | ACCACC | CAAGCAGAAGACGGCATACGAGATACCACCGCTAGGAACGATGAGCCT | 282 |
| | | CCAAC | |
| Ind248 | GACTGC | CAAGCAGAAGACGGCATACGAGATGACTGCGCTAGGAACGATGAGCCT | 283 |
| | | CCAAC | |
| Ind249 | CTACTT | CAAGCAGAAGACGGCATACGAGATCTACTTGCTAGGAACGATGAGCCT | 284 |
| | | CCAAC | |
| Ind250 | TAGCGG | CAAGCAGAAGACGGCATACGAGATTAGCGGGCTAGGAACGATGAGCCT | 285 |
| | | CCAAC | |
| Ind251 | TGTGCG | CAAGCAGAAGACGGCATACGAGATTGTGCGGCTAGGAACGATGAGCCT | 286 |
| | | CCAAC | |
| Ind252 | CTGGCT | CAAGCAGAAGACGGCATACGAGATCTGGCTGCTAGGAACGATGAGCCT | 287 |
| | | CCAAC | |
| Ind253 | CGAGAC | CAAGCAGAAGACGGCATACGAGATCGAGACGCTAGGAACGATGAGCCT | 288 |
| | | CCAAC | |
| Ind254 | GGGATT | CAAGCAGAAGACGGCATACGAGATGGGATTGCTAGGAACGATGAGCCT | 289 |
| | | CCAAC | |
| Ind255 | TACTCC | CAAGCAGAAGACGGCATACGAGATTACTCCCCTAGGAACGATGAGCCT | 290 |
| | | CCAAC | |
| Ind256 | GTGGCA | CAAGCAGAAGACGGCATACGAGATGTGGCAGCTAGGAACGATGAGCCT | 291 |
| | | CCAAC | |
| Ind257 | GTCGTC | CAAGCAGAAGACGGCATACGAGATGTCGTCGCTAGGAACGATGAGCCT | 292 |
| | | CCAAC | |
| Ind258 | GCATTC | CAAGCAGAAGACGGCATACGAGATGCATTCGCTAGGAACGATGAGCCT | 293 |
| | | CCAAC | |
| Ind259 | GAGCGA | CAAGCAGAAGACGGCATACGAGATGAGCGAGCTAGGAACGATGAGCCT | 294 |
| | | CCAAC | |
| Ind260 | AGACAC | CAAGCAGAAGACGGCATACGAGATAGACACGCTAGGAACGATGAGCCT | 295 |
| | | CCAAC | |
| Ind261 | TCTGGG | CAAGCAGAAGACGGCATACGAGATTCTGGGGCTAGGAACGATGAGCCT | 296 |
| | | CCAAC | |
| Ind262 | GCCAGT | CAAGCAGAAGACGGCATACGAGATGCCAGTGCTAGGAACGATGAGCCT | 297 |
| | | CCAAC | |
| Ind263 | CCAGTC | CAAGCAGAAGACGGCATACGAGATCCAGTCGCTAGGAACGATGAGCCT | 298 |
| | | CCAAC | |
| Ind264 | TTGGCC | CAAGCAGAAGACGGCATACGAGATTTGGCCGCTAGGAACGATGAGCCT | 299 |
| | | CCAAC | |
| Ind265 | GTAACA | CAAGCAGAAGACGGCATACGAGATGTAACAGCTAGGAACGATGAGCCT | 300 |
| | | CCAAC | |
| Ind266 | ATTACC | CAAGCAGAAGACGGCATACGAGATATTACCGCTAGGAACGATGAGCCT | 301 |
| | | CCAAC | |
| Ind267 | TCTCCT | CAAGCAGAAGACGGCATACGAGATTCTCCTGCTAGGAACGATGAGCCT | 302 |
| | | CCAAC | |
| Ind268 | AGATCA | CAAGCAGAAGACGGCATACGAGATAGATCAGCTAGGAACGATGAGCCT | 303 |
| | | CCAAC | |
| Ind269 | TCCTAT | CAAGCAGAAGACGGCATACGAGATTCCTATGCTAGGAACGATGAGCCT | 304 |
| | | CCAAC | |
| Ind270 | ACTCGG | CAAGCAGAAGACGGCATACGAGATACTCGGGCTAGGAACGATGAGCCT | 305 |
| | | CCAAC | |
| Ind271 | TGCCAT | CAAGCAGAAGACGGCATACGAGATTGCCATGCTAGGAACGATGAGCCT | 306 |
| | | CCAAC | |
| Ind272 | CATCTC | CAAGCAGAAGACGGCATACGAGATCATCTCGCTAGGAACGATGAGCCT | 307 |
| | | CCAAC | |
| Ind273 | CTTTGA | CAAGCAGAAGACGGCATACGAGATCTTTGAGCTAGGAACGATGAGCCT | 308 |
| | | CCAAC | |
| Ind274 | TCGCAT | CAAGCAGAAGACGGCATACGAGATTCGCATGCTAGGAACGATGAGCCT | 309 |
| | | CCAAC | |
| Ind275 | CACACC | CAAGCAGAAGACGGCATACGAGATCACACCGCTAGGAACGATGAGCCT | 310 |
| | | CCAAC | |
| Ind276 | ACAGAA | CAAGCAGAAGACGGCATACGAGATACAGAAGCTAGGAACGATGAGCCT | 311 |
| | | CCAAC | |
| Ind277 | ATGTCA | CAAGCAGAAGACGGCATACGAGATATGTCAGCTAGGAACGATGAGCCT | 312 |
| | | CCAAC | |
| Ind278 | CTGTCC | CAAGCAGAAGACGGCATACGAGATCTGTCCGCTAGGAACGATGAGCCT | 313 |
| | | CCAAC | |
| Ind279 | CGATCC | CAAGCAGAAGACGGCATACGAGATCGATCCGCTAGGAACGATGAGCCT | 314 |
| | | CCAAC | |
| Ind280 | TGGAGG | CAAGCAGAAGACGGCATACGAGATTGGAGGGCTAGGAACGATGAGCCT | 315 |
| | | CCAAC | |
| Ind281 | CGCCAA | CAAGCAGAAGACGGCATACGAGATCGCCAAGCTAGGAACGATGAGCCT | 316 |
| | | CCAAC | |
| Ind282 | GAATAG | CAAGCAGAAGACGGCATACGAGATGAATAGGCTAGGAACGATGAGCCT | 317 |
| | | CCAAC | |
| Ind283 | CAACGG | CAAGCAGAAGACGGCATACGAGATCAACGGGCTAGGAACGATGAGCCT | 318 |
| | | CCAAC | |
| Ind284 | GCGGAA | CAAGCAGAAGACGGCATACGAGATGCGGAAGCTAGGAACGATGAGCCT | 319 |
| | | CCAAC | |
| Ind285 | TCTGCA | CAAGCAGAAGACGGCATACGAGATTCTGCAGCTAGGAACGATGAGCCT | 320 |
| | | CCAAC | |
| Ind286 | GATGGC | CAAGCAGAAGACGGCATACGAGATGATGGCGCTAGGAACGATGAGCCT | 321 |
| | | CCAAC | |
| Ind287 | CCGATG | CAAGCAGAAGACGGCATACGAGATCCGATGGCTAGGAACGATGAGCCT | 322 |
| | | CCAAC | |
| Ind288 | GATTTC | CAAGCAGAAGACGGCATACGAGATGATTTCGCTAGGAACGATGAGCCT | 323 |
| | | CCAAC | |
| Ind289 | CCAAAC | CAAGCAGAAGACGGCATACGAGATCCAAACGCTAGGAACGATGAGCCT | 324 |
| | | CCAAC | |
| Ind290 | AGGATC | CAAGCAGAAGACGGCATACGAGATAGGATCGCTAGGAACGATGAGCCT | 325 |
| | | CCAAC | |
| Ind291 | CATTCA | CAAGCAGAAGACGGCATACGAGATCATTCAGCTAGGAACGATGAGCCT | 326 |
| | | CCAAC | |
| Ind292 | AGATTG | CAAGCAGAAGACGGCATACGAGATAGATTGGCTAGGAACGATGAGCCT | 327 |
| | | CCAAC | |
| Ind293 | CGAAGC | CAAGCAGAAGACGGCATACGAGATCGAAGCGCTAGGAACGATGAGCCT | 328 |
| | | CCAAC | |
| Ind294 | GGAACG | CAAGCAGAAGACGGCATACGAGATGGAACGGCTAGGAACGATGAGCCT | 329 |
| | | CCAAC | |
| Ind295 | CGACCA | CAAGCAGAAGACGGCATACGAGATCGACCAGCTAGGAACGATGAGCCT | 330 |
| | | CCAAC | |
| Ind296 | AGCTTA | CAAGCAGAAGACGGCATACGAGATAGCTTAGCTAGGAACGATGAGCCT | 331 |
| | | CCAAC | |
| Ind297 | TTCACG | CAAGCAGAAGACGGCATACGAGATTTCACGGCTAGGAACGATGAGCCT | 332 |
| | | CCAAC | |
| Ind298 | CATTAG | CAAGCAGAAGACGGCATACGAGATCATTAGGCTAGGAACGATGAGCCT | 333 |
| | | CCAAC | |
| Ind299 | TAGGAG | CAAGCAGAAGACGGCATACGAGATTAGGAGGCTAGGAACGATGAGCCT | 334 |
| | | CCAAC | |
| Ind300 | CTACCG | CAAGCAGAAGACGGCATACGAGATCTACCGGCTAGGAACGATGAGCCT | 335 |
| | | CCAAC | |
| Ind301 | ATATCC | CAAGCAGAAGACGGCATACGAGATATATCCGCTAGGAACGATGAGCCT | 336 |
| | | CCAAC | |
| Ind302 | AATGGA | CAAGCAGAAGACGGCATACGAGATAATGGAGCTAGGAACGATGAGCCT | 337 |
| | | CCAAC | |
| Ind303 | TTCGAC | CAAGCAGAAGACGGCATACGAGATTTCGACGCTAGGAACGATGAGCCT | 338 |
| | | CCAAC | |
| Ind304 | AACCGG | CAAGCAGAAGACGGCATACGAGATAACCGGGCTAGGAACGATGAGCCT | 339 |
| | | CCAAC | |
| Ind305 | AAGGCC | CAAGCAGAAGACGGCATACGAGATAAGGCCGCTAGGAACGATGAGCCT | 340 |
| | | CCAAC | |
| Ind306 | CGTCCT | CAAGCAGAAGACGGCATACGAGATCGTCCTGCTAGGAACGATGAGCCT | 341 |
| | | CCAAC | |
| Ind307 | AAGAGC | CAAGCAGAAGACGGCATACGAGATAAGAGCGCTAGGAACGATGAGCCT | 342 |
| | | CCAAC | |
| Ind308 | GTGAAA | CAAGCAGAAGACGGCATACGAGATGTGAAAGCTAGGAACGATGAGCCT | 343 |
| | | CCAAC | |
| Ind309 | ACGAAC | CAAGCAGAAGACGGCATACGAGATACGAACGCTAGGAACGATGAGCCT | 344 |
| | | CCAAC | |
| Ind310 | CCGAAA | CAAGCAGAAGACGGCATACGAGATCCGAAAGCTAGGAACGATGAGCCT | 345 |
| | | CCAAC | |
| Ind311 | CAAGGC | CAAGCAGAAGACGGCATACGAGATCAAGGCGCTAGGAACGATGAGCCT | 346 |
| | | CCAAC | |
| Ind312 | ATGTGC | CAAGCAGAAGACGGCATACGAGATATGTGCGCTAGGAACGATGAGCCT | 347 |
| | | CCAAC | |
| Ind313 | CCCTTG | CAAGCAGAAGACGGCATACGAGATCCCTTGGCTAGGAACGATGAGCCT | 348 |
| | | CCAAC | |
| Ind314 | GAGAAG | CAAGCAGAAGACGGCATACGAGATGAGAAGGCTAGGAACGATGAGCCT | 349 |
| | | CCAAC | |
| Ind315 | GTAGTT | CAAGCAGAAGACGGCATACGAGATGTAGTTGCTAGGAACGATGAGCCT | 350 |
| | | CCAAC | |
| Ind316 | TGGTGC | CAAGCAGAAGACGGCATACGAGATTGGTGCGCTAGGAACGATGAGCCT | 351 |
| | | CCAAC | |
| Ind317 | GACTAT | CAAGCAGAAGACGGCATACGAGATGACTATGCTAGGAACGATGAGCCT | 352 |
| | | CCAAC | |
| Ind318 | CTCGCA | CAAGCAGAAGACGGCATACGAGATCTCGCAGCTAGGAACGATGAGCCT | 353 |
| | | CCAAC | |
| Ind319 | TCTTGT | CAAGCAGAAGACGGCATACGAGATTCTTGTGCTAGGAACGATGAGCCT | 354 |
| | | CCAAC | |
| Ind320 | GCACAA | CAAGCAGAAGACGGCATACGAGATGCACAAGCTAGGAACGATGAGCCT | 355 |
| | | CCAAC | |
| Ind321 | CCTTTA | CAAGCAGAAGACGGCATACGAGATCCTTTAGCTAGGAACGATGAGCCT | 356 |
| | | CCAAC | |
| Ind322 | ACGTTG | CAAGCAGAAGACGGCATACGAGATACGTTGGCTAGGAACGATGAGCCT | 357 |
| | | CCAAC | |
| Ind323 | AAGCGT | CAAGCAGAAGACGGCATACGAGATAAGCGTGCTAGGAACGATGAGCCT | 358 |
| | | CCAAC | |
| Ind324 | AGGCAA | CAAGCAGAAGACGGCATACGAGATAGGCAAGCTAGGAACGATGAGCCT | 359 |
| | | CCAAC | |
| Ind325 | TGAGCC | CAAGCAGAAGACGGCATACGAGATTGAGCCGCTAGGAACGATGAGCCT | 360 |
| | | CCAAC | |
| Ind326 | TGAGAA | CAAGCAGAAGACGGCATACGAGATTGAGAAGCTAGGAACGATGAGCCT | 361 |
| | | CCAAC | |
| Ind327 | GTACAG | CAAGCAGAAGACGGCATACGAGATGTACAGGCTAGGAACGATGAGCCT | 362 |
| | | CCAAC | |
| Ind328 | ACGGAG | CAAGCAGAAGACGGCATACGAGATACGGAGGCTAGGAACGATGAGCCT | 363 |
| | | CCAAC | |
| Ind329 | ACATAC | CAAGCAGAAGACGGCATACGAGATACATACGCTAGGAACGATGAGCCT | 364 |
| | | CCAAC | |
| Ind330 | CAGCCA | CAAGCAGAAGACGGCATACGAGATCAGCCAGCTAGGAACGATGAGCCT | 365 |
| | | CCAAC | |
| Ind331 | TGTCAA | CAAGCAGAAGACGGCATACGAGATTGTCAAGCTAGGAACGATGAGCCT | 366 |
| | | CCAAC | |
| Ind332 | TATTGG | CAAGCAGAAGACGGCATACGAGATTATTGGGCTAGGAACGATGAGCCT | 367 |
| | | CCAAC | |
| Ind333 | AGACCG | CAAGCAGAAGACGGCATACGAGATAGACCGGCTAGGAACGATGAGCCT | 368 |
| | | CCAAC | |
| Ind334 | GCCAAC | CAAGCAGAAGACGGCATACGAGATGCCAACGCTAGGAACGATGAGCCT | 369 |
| | | CCAAC | |
| Ind335 | ATGTAG | CAAGCAGAAGACGGCATACGAGATATGTAGGCTAGGAACGATGAGCCT | 370 |
| | | CCAAC | |
| Ind336 | CGCTAC | CAAGCAGAAGACGGCATACGAGATCGCTACGCTAGGAACGATGAGCCT | 371 |
| | | CCAAC | |
| Ind337 | CACTCG | CAAGCAGAAGACGGCATACGAGATCACTCGGCTAGGAACGATGAGCCT | 372 |
| | | CCAAC | |
| Ind338 | GCTATT | CAAGCAGAAGACGGCATACGAGATGCTATTGCTAGGAACGATGAGCCT | 373 |
| | | CCAAC | |
| Ind339 | CTCCAC | CAAGCAGAAGACGGCATACGAGATCTCCACGCTAGGAACGATGAGCCT | 374 |
| | | CCAAC | |
| Ind340 | GTTAGC | CAAGCAGAAGACGGCATACGAGATGTTAGCGCTAGGAACGATGAGCCT | 375 |
| | | CCAAC | |
| Ind341 | AAACCT | CAAGCAGAAGACGGCATACGAGATAAACCTGCTAGGAACGATGAGCCT | 376 |
| | | CCAAC | |
| Ind342 | CTAGAT | CAAGCAGAAGACGGCATACGAGATCTAGATGCTAGGAACGATGAGCCT | 377 |
| | | CCAAC | |
| Ind343 | ACCGTC | CAAGCAGAAGACGGCATACGAGATACCGTCGCTAGGAACGATGAGCCT | 378 |
| | | CCAAC | |
| Ind344 | TCACCC | CAAGCAGAAGACGGCATACGAGATTCACCCGCTAGGAACGATGAGCCT | 379 |
| | | CCAAC | |
| Ind345 | GAAGAT | CAAGCAGAAGACGGCATACGAGATGAAGATGCTAGGAACGATGAGCCT | 380 |
| | | CCAAC | |
| Ind346 | TGGCTC | CAAGCAGAAGACGGCATACGAGATTGGCTCGCTAGGAACGATGAGCCT | 381 |
| | | CCAAC | |
| Ind347 | GGTTAT | CAAGCAGAAGACGGCATACGAGATGGTTATGCTAGGAACGATGAGCCT | 382 |
| | | CCAAC | |
| Ind348 | ACACTT | CAAGCAGAAGACGGCATACGAGATACACTTGCTAGGAACGATGAGCCT | 383 |
| | | CCAAC | |
| Ind349 | GGAAGA | CAAGCAGAAGACGGCATACGAGATGGAAGAGCTAGGAACGATGAGCCT | 384 |
| | | CCAAC | |
| Ind350 | TACGTG | CAAGCAGAAGACGGCATACGAGATTACGIGGCTAGCAACGATGAGCCT | 385 |
| | | CCAAC | |
| Ind351 | CTTGAC | CAAGCAGAAGACGGCATACGAGATCTTGACGCTAGGAACGATGAGCCT | 386 |
| | | CCAAC | |
| Ind352 | ATGCCC | CAAGCAGAAGACGGCATACGAGATATGCCCGCTAGGAACGATGAGCCT | 387 |
| | | CCAAC | |
| Ind353 | ATTCAC | CAAGCAGAAGACGGCATACGAGATATTCACGCTAGGAACGATGAGCCT | 388 |
| | | CCAAC | |
| Ind354 | GCTTAC | CAAGCAGAAGACGGCATACGAGATGCTTACGCTAGGAACGATGAGCCT | 389 |
| | | CCAAC | |
| Ind355 | TTCTGC | CAAGCAGAAGACGGCATACGAGATTTCTGCGCTAGGAACGATGAGCCT | 390 |
| | | CCAAC | |
| Ind356 | TGTATG | CAAGCAGAAGACGGCATACGAGATTGTATGGCTAGGAACGATGAGCCT | 391 |
| | | CCAAC | |
| Ind357 | TGACGC | CAAGCAGAAGACGGCATACGAGATTGACGCGCTAGGAACGATGAGCCT | 392 |
| | | CCAAC | |
| Ind358 | GTGTTA | CAAGCAGAAGACGGCATACGAGATGTGTTAGCTAGGAACGATGAGCCT | 393 |
| | | CCAAC | |
| Ind359 | CATCGA | CAAGCAGAAGACGGCATACGAGATCATCGAGCTAGGAACGATGAGCCT | 394 |
| | | CCAAC | |
| Ind360 | TGAGGT | CAAGCAGAAGACGGCATACGAGATTGAGGTGCTAGGAACGATGAGCCT | 395 |
| | | CCAAC | |
| Ind361 | GGTCCA | CAAGCAGAAGACGGCATACGAGATGGTCCAGCTAGGAACGATGAGCCT | 396 |
| | | CCAAC | |
| Ind362 | TATGCT | CAAGCAGAAGACGGCATACGAGATTATGCTGCTAGGAACGATGAGCCT | 397 |
| | | CCAAC | |
| Ind363 | TTCATC | CAAGCAGAAGACGGCATACGAGATTTCATCGCTAGGAACGATGAGCCT | 398 |
| | | CCAAC | |
| Ind364 | CCTCTG | CAAGCAGAAGACGGCATACGAGATCCTCTGGCTAGGAACGATGAGCCT | 399 |
| | | CCAAC | |
| Ind365 | CCAAGG | CAAGCAGAAGACGGCATACGAGATCCAAGGGCTAGGAACGATGAGCCT | 400 |
| | | CCAAC | |
| Ind366 | TAATGC | CAAGCAGAAGACGGCATACGAGATTAATGCGCTAGGAACGATGAGCCT | 401 |
| | | CCAAC | |
| Ind367 | AGGGCA | CAAGCAGAAGACGGCATACGAGATAGGGCAGCTAGGAACGATGAGCCT | 402 |
| | | CCAAC | |
| Ind368 | TAGAGA | CAAGCAGAAGACGGCATACGAGATTAGAGAGCTAGGAACGATGAGCCT | 403 |
| | | CCAAC | |
| Ind369 | AGCACA | CAAGCAGAAGACGGCATACGAGATAGCACAGCTAGGAACGATGAGCCT | 404 |
| | | CCAAC | |
| Ind370 | AGAGTC | CAAGCAGAAGACGGCATACGAGATAGAGTCGCTAGGAACGATGAGCCT | 405 |
| | | CCAAC | |
| Ind371 | ACTCAA | CAAGCAGAAGACGGCATACGAGATACTCAAGCTAGGAACGATGAGCCT | 406 |
| | | CCAAC | |
| Ind372 | GTGACG | CAAGCAGAAGACGGCATACGAGATGTGACGGCTAGGAACGATGAGCCT | 407 |
| | | CCAAC | |
| Ind373 | GGTAGG | CAAGCAGAAGACGGCATACGAGATGGTAGGGCTAGGAACGATGAGCCT | 408 |
| | | CCAAC | |
| Ind374 | CTGAAT | CAAGCAGAAGACGGCATACGAGATCTGAATGCTAGGAACGATGAGCCT | 409 |
| | | CCAAC | |
| Ind375 | GTCTAC | CAAGCAGAAGACGGCATACGAGATGTCTACGCTAGGAACGATGAGCCT | 410 |
| | | CCAAC | |
| Ind376 | TCGGAC | CAAGCAGAAGACGGCATACGAGATTCGGACGCTAGGAACGATGAGCCT | 411 |
| | | CCAAC | |
| Ind377 | AACTAC | CAAGCAGAAGACGGCATACGAGATAACTACGCTAGGAACGATGAGCCT | 412 |
| | | CCAAC | |
| Ind378 | AAGGTT | CAAGCAGAAGACGGCATACGAGATAAGGTTGCTAGGAACGATGAGCCT | 413 |
| | | CCAAC | |
| Ind379 | AGGGAC | CAAGCAGAAGACGGCATACGAGATAGGGACGCTAGGAACGATGAGCCT | 414 |
| | | CCAAC | |
| Ind380 | ACGCGA | CAAGCAGAAGACGGCATACGAGATACGCGAGCTAGGAACGATGAGCCT | 415 |
| | | CCAAC | |
| Ind381 | TTGCTA | CAAGCAGAAGACGGCATACGAGATTTGCTAGCTAGGAACGATGAGCCT | 416 |
| | | CCAAC | |
| Ind382 | ATAACG | CAAGCAGAAGACGGCATACGAGATATAACGGCTAGGAACGATGAGCCT | 417 |
| | | CCAAC | |
| Ind383 | CCGGTA | CAAGCAGAAGACGGCATACGAGATCCGGTAGCTAGGAACGATGAGCCT | 418 |
| | | CCAAC | |
| Ind384 | AGCTAG | CAAGCAGAAGACGGCATACGAGATAGCTAGGCTAGGAACGATGAGCCT | 419 |
| | | CCAAC | |

Bisulfite Read Mapping and Data Analysis

The reference genome was computationally converted by changing all cytosines to thymines on the two strands separately. Sequencing reads were encoded by (i) predicting the mapping orientation, (ii) converting all predicted forward mapping reads by changing all cytosines to thymines and converting all predicted reverse complementary mapping reads by changing all guanines to adenines, the original reads are maintained. The bisulfite reads were then mapped to the converted reference separately using SOAP2Align with the parameters r=0 (report uniquely mapped reads only), v=2 (number of allowable mismatch), paired-end: m=0 (minimal insert size), x=400 (maximum insert size). Alignment files were then combined, and one alignment per read was selected. Original C calls were placed back into the alignment information. Alignments were then converted to pileup format using SamTools. Raw SNPs and methylation frequency files were computed from pileup counts. Methylation frequencies were called using a method described previously (Deng, J. et al. (2009) Nat. Biotechnol. 27: 353-360).

Correlation of Methylation Between Two Samples

To check whether methylation levels were similar between two samples, the Pearson's correlation was calculated on all CpG sites characterizable in both samples. First, a list of CpG sites with read depth of at least ten in both samples was generated. The methylation frequencies at these sites were obtained from bisReadMapper output and input into the statistical package R. Finally, Pearson's correlation for the two samples was computed using the cor( ) function.

Analysis of Methylation

From the bisReadMapper output, the raw read counts showing methylation and lack of methylation were assembled for each line. Using these counts, a Fisher Exact Test with Benjamini-Hochberg multiple testing correction (false discovery rate=0.01) was carried out on each CpG site with minimum 10× depth coverage. This resulted in a set of DMSs between the two lines; at each of these sites, the methylation had at least 0.1 methylation level of difference. Technical replicates did not show any differential methylation, and different cell types showed a large extent of differential methylation (˜33%).

### Results and Discussion

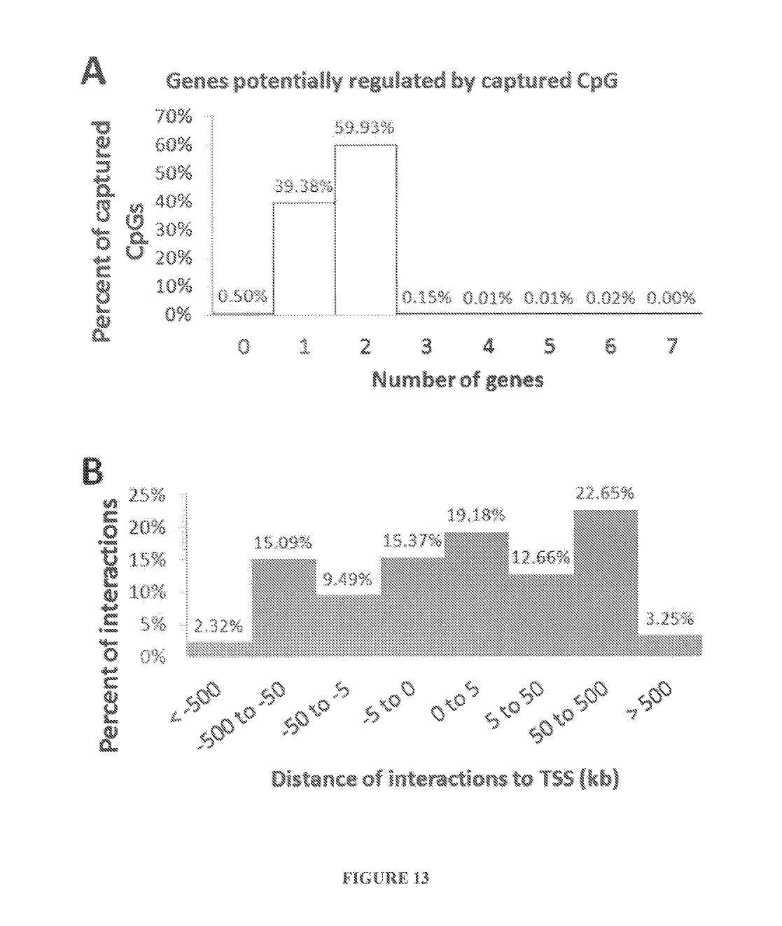

These probes were used to analyze H1 embryonic stem cells (H1 ESCs), PGP1 fibroblasts and two technical replicates of PGP 1 fibroblast-derived induced pluripotent stem cells (PGP1-iPSCs). For each sample, on average ˜3.66 gigabases were sequenced and measured methylation for an average of 480,904 CpG sites. To assess whether these data could be used to identify potential epigenetic regulation of transcription, the Genomic Regions Enrichment of Annotations Tool (“GREAT”; McLean, C. Y. et al. (2010) Nat. Biotechnol. 28: 495-501) was used to predict the cis-regulatory potential of regions around captured CpG sites. In total, the padlock probes captured CpG sites in regions predicted to regulate 98% of RefSeq genes (FIG. 13).

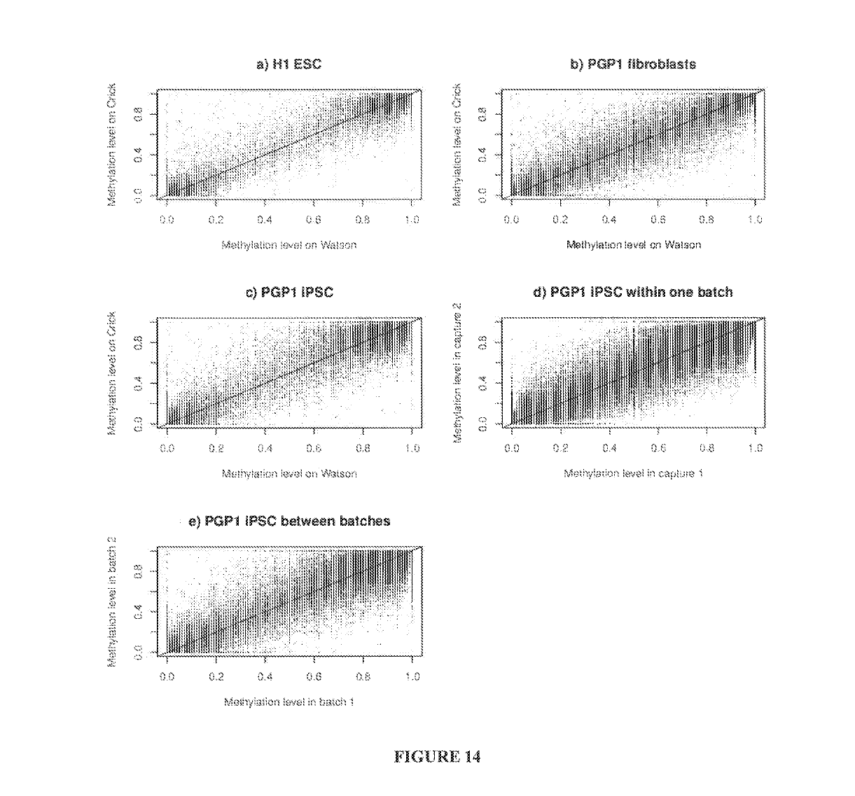

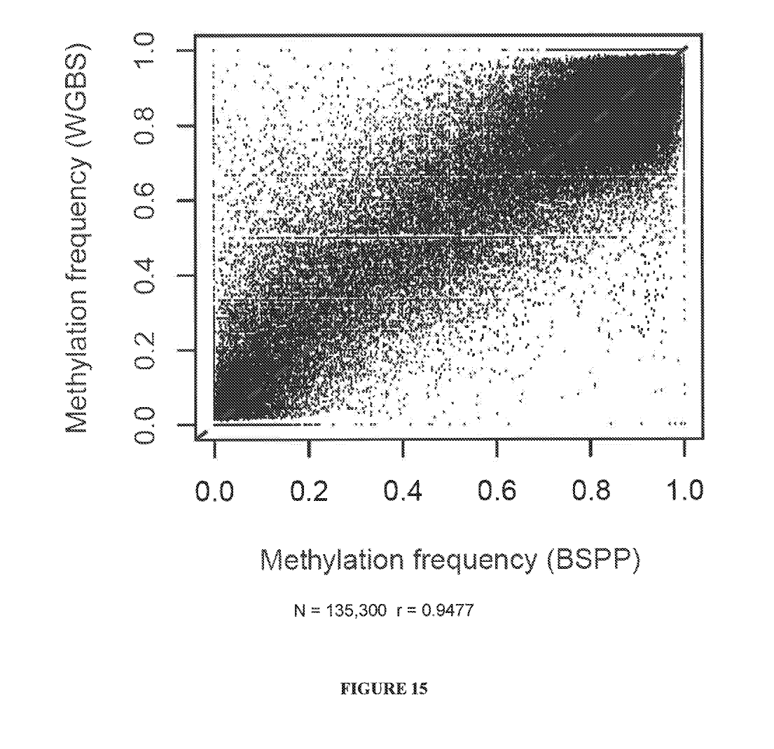

The data generated with BSPPs accurately represented the methylation status of the target regions. Methylation levels for the two technical replicates of PGP1-iPSCs were consistent both within a single batch and between separate batches (Pearson's correlation coefficient R=0.97-0.98, FIG. 14A-14B). Additionally, when we compared methylation levels between technical replicates, no CpG site was different by a Fisher Exact Test with Benjamini-Hochberg multiple testing correction (false discovery rate=0.01, n=439,090). In comparison, large fractions of sites were differentially methylated owing to either the process of nuclear reprogramming (27.9% DMSs between PGP1-iPSCs and PGP1 fibroblasts) or the difference in cell type (31.3% DMSs between PGP1 fibroblasts and H1 ESCs) with the same criteria (false discovery rate=0.01, n=444,111 and 359,290, respectively). Our BSPP results with H1 ESCs were consistent with the published whole-genome sequencing of bisulfite-converted DNA12 (Pearson's correlation coefficient R=0.95, FIG. 15).

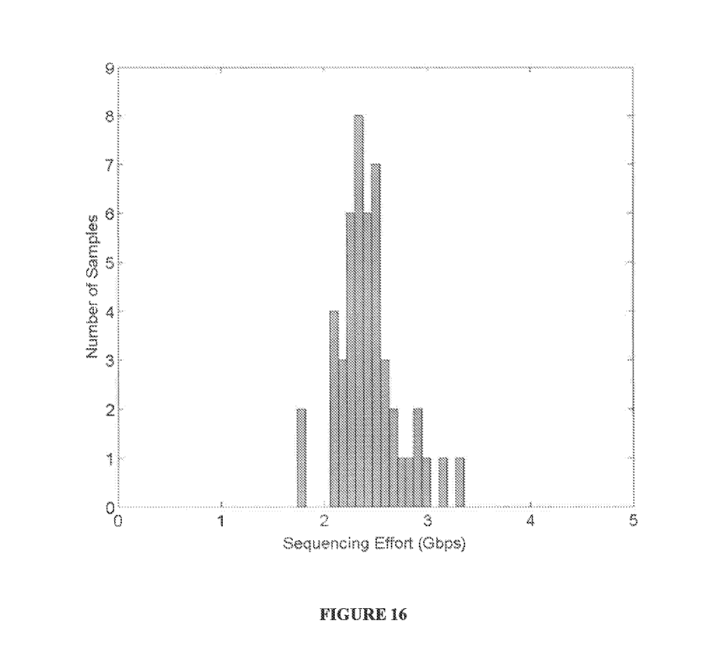

This assay has very low technical variability. The assay was performed on over 150 samples in 96-well plates; the yield for each was similar (FIG. 16). Approximately 10% of CpG sites were targeted separately on each strand, allowing low-quality datasets with poor correlation between these built-in technical replicates to be identified (FIG. 14C-14E). As our BSPP assay measures absolute methylation, no normalization is necessary as long as the internal replicates are consistent. Therefore, many datasets, even those generated in different laboratories, can be directly compared without batch effects, which is important for case-control studies on large samples or for meta-analyses. Additionally, the SNP-calling feature of bisReadMapper allowed for characterization of roughly 20,000 SNPs for each sample with an accuracy of 96% or better. This allowed unambiguous tracking of samples, which is crucial for projects involving large sample sizes.

The library-free BSPP method is flexible for different study designs. Whereas the genome-scale probe set allows global profiling on thousands of samples, a focused assay is necessary to follow up on tens to hundreds of candidate regions identified in genome-scale scanning. Such an assay needs to be customizable to different genomic targets, scalable to a very large sample size (1,000-100,000 samples), and inexpensive. To additionally test the flexibility, a second set of 3,918 probes (LC Sciences) was designed to evaluate the methylation state 1 kbp upstream and downstream of 120 genomic regions previously known and confirmed by BSPP to carry aberrant methylation in induced pluripotent stem cells (Lister, R. et al. (2011) Nature 471(7336): 68-73). Even with shorter capturing sequences (40 bp total for capturing arms rather than 50 bp on average, FIG. 17) and a 100-fold smaller target size, an average of 56% of mappable bases were on-target, equivalent to an enrichment factor of ˜6,500. With the data from three cell lines (H1 ESCs, PGP1 fibroblasts and PGP1-iPSCs) regions of aberrant methylation were identified in induced pluripotent stem cells and it was found that aberrant methylation continues further upstream and downstream than observed previously.

This analysis demonstrated that a focused probe set can be used to validate specific regions of interest identified in global scanning using either our genome-wide probe set or other methods.

### Example 3

### Improvements to ppDesigner

Several key improvements were made to both the probe design algorithm and the ppDesigner software implementing it. These changes allow for improved generation of low-bias padlock probes for bisulfite sequencing applications and additional customizability in probe parameters such that probes can be synthesized from additional oligonucleotide vendors and utilized in additional experimental protocols.

The previous version of the probe design algorithm utilized one neural network to generate the optimal set of padlock probes for both normal and bisulfite-converted DNA targets. However, this network was specifically designed using results from probe experiments without bisulfite conversion. In the improved algorithm, a second neural network was added specifically for bisulfite-converted DNA based solely on bisulfite probe experiments. This network contains two hidden layers with 10 and 12 nodes, respectively (FIG. 18), and accepts 25 pieces of information as input rather than 7. These 25 factors (Table 8) were specifically chosen for their ability to influence padlock probe capturing efficiency on bisulfite-converted DNA.

TABLE 8
Factors used by neural network to predict probe capturing efficiency

| Factors used in Bisulfite Padlock | Factors used in Genomic Padlock |
|---|---|
| Probe Design | Probe Design |
| Extension Arm A% | Target Folding Energy |
| Extension Arm G% | Target GC Content |
| Target A% | Ligation Arm Melting Temperature |
| Target T% | Extension Arm Melting Temperature |
| Target G% | Ligation Arm Length |
| Number of ″GG″ Dinucleotides in | Extension Arm Length |
| Ligation Arm | |
| Number of ″AT″ Dinucleotides in | Target Length |
| Extension Arm | |
| Number of ″GG″ Dinucleotides in | |
| Extension Arm | |
| Number of ″AA″ Dinucleotides in | |
| Target | |
| Number of ″AT″ Dinucleotides in | |
| Target | |
| Number of ″TA″ Dinucleotides in | |
| Target | |
| Number of ″GT″ Dinucleotides in | |
| Target | |
| Number of ″GA″ Dinucleotides in | |
| Target | |
| Ligation Arm Terminal Dinucleotide | |
| Extension Arm Terminal | |
| Dinucleotide | |
| Target 5′ Terminal Dinucleotide | |
| Ligation Arm Length | |
| Target Length | |
| Overall Melting Temperature | |

Significant new factors include the counts of each DNA dinucleotide in the target region and the dinucleotides surrounding the ligation site. The use of two neural networks for the two separate capturing conditions allows for a more efficient prediction of probe efficiency, reducing capturing bias and experimental cost.

In addition, new user interface improvements were implemented to better facilitate these protocols. Several simple “default” setting profiles were added to allow the probe designer to more easily account for limitations in oligonucleotide synthesis from various vendors (including Agilent Technologies and LC Sciences). Additional parameters were also added, allowing for easy implementation of the new library-free padlock probe protocol, including allowing the user to set a fixed total arm size and providing the specialized library-free linker sequence.

What is claimed is:

1. A method of designing probes or primers for sequencing a target nucleic acid molecule, comprising:
selecting one or more inputs associated with efficiency of the probe or primer;
selecting a target nucleic acid sequence;
generating a first library of probe or primer sequences that comprise a target capturing sequence that is complementary to the target nucleic acid sequence;
determining the efficiency of each probe or primer sequence in the first library by using an algorithm comprising the one or more selected inputs;
ranking the probe or primer sequences in the first library by efficiency;
extracting the probe or primer sequences having the highest efficiency to generate a second library; and
adding a linker sequence to each of the probe or primer sequences in the second library.

2. The method of claim 1, further comprising synthesizing the probe or primer.

3. The method of claim 1, wherein the probe is a padlock probe.

4. The method of claim 1, wherein the one or more inputs comprise target length, target folding energy, target GC content, extension arm A %, extension Arm G %, target A %, target T %, target G %, number of “GG” dinucleotides in ligation arm, number of “AT” dinucleotides in extension arm, number of “GG” dinucleotides in extension arm, number of “AA” dinucleotides in target, number of “AT” dinucleotides in target, number of “TA” dinucleotides in target, number of “GT” dinucleotides in target, number of “GA” dinucleotides in target, ligation arm terminal dinucleotide, extension arm terminal dinucleotide, target 5′ terminal dinucleotide, ligation arm melting temperature, extension arm melting temperature, ligation arm length, extension arm length, local single-stranded folding energy of the target, and dinucleotides present at the extension site and ligation site during probe capture.

5. The method of claim 1, wherein the target nucleic acid sequence is derived from a human.

6. The method of claim 1, wherein the target-capturing sequence includes a ligation arm and an extension arm.

7. The method of claim 1, wherein the target-capturing sequence contains one or more CpG dinucleotides.

8. The method of claim 6, wherein the target-capturing sequences in the first library contain all possible methylation state combinations of the one or more CpG dinucleotides.

9. The method of claim 5, wherein the extension arm comprises one or more priming sites for amplification of the target nucleic acid sequence.

10. The method of claim 8, wherein the one or more priming sites are universal priming sites.

11. The method of claim 1, wherein the target capturing sequence includes one or more restriction sites.

12. The method of claim 1, wherein the algorithm comprises one or more neural networks.

13. The method of claim 12, wherein the one or more neural networks comprise the one or more inputs.

14. The method of claim 13, wherein the one or more neural networks comprise seven or more inputs.

15. The method of claim 1, wherein the method further comprises, after the extracting, pooling the non-extracted probe or primer sequences and repeating the steps of generating the library of probe or primer sequences, determining the efficiency of each probe or primer sequence by using the algorithm, ranking the probe or primer sequences by efficiency, and extracting the probe or primer sequences having the highest efficiency.

16. The method of claim 1, wherein the linker sequence is common to each probe or primer sequence in the second library.

17. An apparatus comprising at least one processor and at least one memory including code which when executed by the at least one processor provides operations comprising:
selecting one or more inputs associated with efficiency of the probe or primer;
selecting a target nucleic acid sequence;
generating a first library of probe or primer sequences that comprise a target capturing sequence that is complementary to the target nucleic acid sequence;
determining the efficiency of each probe or primer sequence in the first library by using an algorithm comprising the one or more selected inputs;
ranking the probe or primer sequences in the first library by efficiency;
extracting the probe or primer sequences having the highest efficiency to generate a second library; and
adding a linker sequence to each of the probe or primer sequences in the second library.

18. The apparatus of claim 17, wherein the operations further comprise synthesizing the probe or primer.

19. The apparatus of claim 17, wherein the probe is a padlock probe.

20. The apparatus of claim 17, wherein the one or more inputs comprise target length, target folding energy, target GC content, extension arm A %, extension Arm G %, target A %, target T %, target G %, number of “GG” dinucleotides in ligation arm, number of “AT” dinucleotides in extension arm, number of “GG” dinucleotides in extension arm, number of “AA” dinucleotides in target, number of “AT” dinucleotides in target, number of “TA” dinucleotides in target, number of “GT” dinucleotides in target, number of “GA” dinucleotides in target, ligation arm terminal dinucleotide, extension arm terminal dinucleotide, target 5′ terminal dinucleotide, ligation arm melting temperature, extension arm melting temperature, ligation arm length, extension arm length, local single-stranded folding energy of the target, and dinucleotides present at the extension site and ligation site during probe capture.

21. The apparatus of claim 17, wherein the algorithm comprises one or more neural networks.

22. The apparatus of claim 21, wherein the one or more neural networks comprise the one or more inputs.

23. The apparatus of claim 22, wherein the one or more neural networks comprise seven or more inputs.

24. The apparatus of claim 17, wherein the operations further comprise, after the extracting, pooling the non-extracted probe or primer sequences and repeating the steps of generating the library of probe or primer sequences, determining the efficiency of each probe or primer sequence by using the algorithm, ranking the probe or primer sequences by efficiency, and extracting the probe or primer sequences having the highest efficiency.

25. A computer-readable storage medium including code, which when executed by at least one processor provides operations comprising:
selecting one or more inputs associated with efficiency of the probe or primer;
selecting a target nucleic acid sequence;
generating a first library of probe or primer sequences that comprise a target capturing sequence that is complementary to the target nucleic acid sequence;
determining the efficiency of each probe or primer sequence in the first library by using an algorithm comprising the one or more selected inputs;
ranking the probe or primer sequences in the first library by efficiency;
extracting the probe or primer sequences having the highest efficiency to generate a second library; and
adding a linker sequence to each of the probe or primer sequences in the second library.

26. The computer-readable storage medium of claim 25, wherein the operations further comprise synthesizing the probe or primer.

27. The computer-readable storage medium of claim 25, wherein the probe is a padlock probe.

28. The computer-readable storage medium of claim 25, wherein the one or more inputs comprise target length, target folding energy, target GC content, extension arm A %, extension Arm G %, target A %, target T %, target G %, number of “GG” dinucleotides in ligation arm, number of “AT” dinucleotides in extension arm, number of “GG” dinucleotides in extension arm, number of “AA” dinucleotides in target, number of “AT” dinucleotides in target, number of “TA” dinucleotides in target, number of “GT” dinucleotides in target, number of “GA” dinucleotides in target, ligation arm terminal dinucleotide, extension arm terminal dinucleotide, target 5′ terminal dinucleotide, ligation arm melting temperature, extension arm melting temperature, ligation arm length, extension arm length, local single-stranded folding energy of the target, and dinucleotides present at the extension site and ligation site during probe capture.

29. The computer-readable storage medium of claim 25, wherein the algorithm comprises one or more neural networks.

30. The computer-readable storage medium of claim 29, wherein the one or more neural networks comprise the one or more inputs.

31. The computer-readable storage medium of claim 30, wherein the one or more neural networks comprise seven or more inputs.

32. The computer-readable storage medium of claim 25, wherein the operations further comprise, after the extracting, pooling the non-extracted probe or primer sequences and repeating the steps of generating the library of probe or primer sequences, determining the efficiency of each probe or primer sequence by using the algorithm, ranking the probe or primer sequences by efficiency, and extracting the probe or primer sequences having the highest efficiency.


SEQUENCE LISTING (430 sequences)

SEQ ID NO: 1 - DNA, 21 bp, Artificial Sequence
  Description of Artificial Sequence Synthetic primer
gtagactggaagagcactgtu

SEQ ID NO: 2 - DNA, 20 bp, Artificial Sequence
  Description of Artificial Sequence Synthetic primer
tagcctcatgcgtatccgat

SEQ ID NO: 3 - DNA, 14 bp, Artificial Sequence
  Description of Artificial Sequence Synthetic primer
atgcgtatccgatc

SEQ ID NO: 4 - DNA, 45 bp, Artificial Sequence
  Description of Artificial Sequence Synthetic primer
aatgatacggcgaccaccgacactctctgcagatgttatcgaggt

SEQ ID NO: 5 - DNA, 49 bp, Artificial Sequence
  Description of Artificial Sequence Synthetic primer
caagcagaagacggcatacgagctcttcacgcagctgaataggaacgat

SEQ ID NO: 6 - DNA, 21 bp, Artificial Sequence
  Description of Artificial Sequence Synthetic primer
cagatgttatcgaggtccgac

SEQ ID NO: 7 - DNA, 19 bp, Artificial Sequence
  Description of Artificial Sequence Synthetic primer
ggaacgatgagcctccaac

SEQ ID NO: 8 - DNA, 20 bp, Artificial Sequence
  Description of Artificial Sequence Synthetic primer
gggtcatatcggtcactgtu

SEQ ID NO: 9 - DNA, 20 bp, Artificial Sequence
  Description of Artificial Sequence Synthetic primer
cacgggtagtgtgtatcctg

SEQ ID NO: 10 - DNA, 13 bp, Artificial Sequence
  Description of Artificial Sequence Synthetic primer
gtgtatcctgatc

SEQ ID NO: 11 - DNA, 56 bp, Artificial Sequence
  Description of Artificial Sequence Synthetic primer
aatgatacggcgaccaccgagatctacaccactctcagatgttatcgaggtccgac

SEQ ID NO: 12 - DNA, 21 bp, Artificial Sequence
  Description of Artificial Sequence Synthetic primer
cagatgttatcgaggtccgac

SEQ ID NO: 13 - DNA, 19 bp, Artificial Sequence
  Description of Artificial Sequence Synthetic primer
ggaacgatgagcctccaac

SEQ ID NO: 14 - DNA, 52 bp, Artificial Sequence
  Description of Artificial Sequence Synthetic primer
aatgatacggcgaccaccgagatctacactctttccctacacgacgctcttc

SEQ ID NO: 15 - DNA, 35 bp, Artificial Sequence
  Description of Artificial Sequence Synthetic primer
acactctttccctacacgacgctcttccgatctnn

SEQ ID NO: 16 - DNA, 33 bp, Artificial Sequence
  Description of Artificial Sequence Synthetic primer
agatcggaagagcggttcagcaggaatgccgag

SEQ ID NO: 17 - DNA, 32 bp, Artificial Sequence
  Description of Artificial Sequence Synthetic primer
tacaccactctcagatgttatcgaggtccgac

SEQ ID NO: 18 - DNA, 23 bp, Artificial Sequence
  Description of Artificial Sequence Synthetic primer
gctaggaacgatgagcctccaac

SEQ ID NO: 19 - DNA, 23 bp, Artificial Sequence
  Description of Artificial Sequence Synthetic primer
gttggaggctcatcgttcctagc

SEQ ID NO: 20 - DNA, 20 bp, Artificial Sequence
  Description of Artificial Sequence Synthetic primer
tgcctaggaccggatcaact

SEQ ID NO: 21 - DNA, 20 bp, Artificial Sequence
  Description of Artificial Sequence Synthetic primer
gagcttcggttcacgcaatg

SEQ ID NO: 22 - DNA, 20 bp, Artificial Sequence
  Description of Artificial Sequence Synthetic primer
gcacgatccgacggtagtgt

SEQ ID NO: 23 - DNA, 20 bp, Artificial Sequence
  Description of Artificial Sequence Synthetic primer
ccgtaatcgggaagctgaag

SEQ ID NO: 24 - DNA, 54 bp, Artificial Sequence
  Description of Artificial Sequence Synthetic primer
caagcagaagacggcatacgagatgatctgcggtctgccatccgacggtagtgt

SEQ ID NO: 25 - DNA, 54 bp, Artificial Sequence
  Description of Artificial Sequence Synthetic primer
caagcagaagacggcatacgagatcgtagtcggtctgccatccgacggtagtgt

SEQ ID NO: 26 - DNA, 54 bp, Artificial Sequence
  Description of Artificial Sequence Synthetic primer
caagcagaagacggcatacgagataataggcggtctgccatccgacggtagtgt

SEQ ID NO: 27 - DNA, 48 bp, Artificial Sequence
  Description of Artificial Sequence Synthetic primer
aatgatacggcgaccaccgagatctacacgcctatcgggaagctgaag

SEQ ID NO: 28 - DNA, 48 bp, Artificial Sequence
  Description of Artificial Sequence Synthetic primer
aatgatacggcgaccaccgagatctacacgcctatccgacggtagtgt

SEQ ID NO: 29 - DNA, 54 bp, Artificial Sequence
  Description of Artificial Sequence Synthetic primer
caagcagaagacggcatacgagatgatctgcggtctgccatcgggaagctgaag

SEQ ID NO: 30 - DNA, 54 bp, Artificial Sequence
  Description of Artificial Sequence Synthetic primer
caagcagaagacggcatacgagatcgtagtcggtctgccatcgggaagctgaag

SEQ ID NO: 31 - DNA, 54 bp, Artificial Sequence
  Description of Artificial Sequence Synthetic primer
caagcagaagacggcatacgagataataggcggtctgccatcgggaagctgaag

SEQ ID NO: 32 - DNA, 24 bp, Artificial Sequence
  Description of Artificial Sequence Synthetic primer
tacacgcctatcgggaagctgaag

SEQ ID NO: 33 - DNA, 24 bp, Artificial Sequence
  Description of Artificial Sequence Synthetic primer
acactaccgtcggatggcagaccg

SEQ ID NO: 34 - DNA, 24 bp, Artificial Sequence
  Description of Artificial Sequence Synthetic primer
tacacgcctatccgacggtagtgt

SEQ ID NO: 35 - DNA, 24 bp, Artificial Sequence
  Description of Artificial Sequence Synthetic primer
cttcagcttcccgatggcagaccg

SEQ ID NO: 36 - DNA, 53 bp, Artificial Sequence
  Description of Artificial Sequence Synthetic primer
caagcagaagacggcatacgagatcgtgatgctaggaacgatgagcctccaac

SEQ ID NO: 37 - DNA, 53 bp, Artificial Sequence
  Description of Artificial Sequence Synthetic primer
caagcagaagacggcatacgagatacatcggctaggaacgatgagcctccaac

SEQ ID NO: 38 - DNA, 53 bp, Artificial Sequence
  Description of Artificial Sequence Synthetic primer
caagcagaagacggcatacgagatgcctaagctaggaacgatgagcctccaac

SEQ ID NO: 39 - DNA, 53 bp, Artificial Sequence
  Description of Artificial Sequence Synthetic primer
caagcagaagacggcatacgagattggtcagctaggaacgatgagcctccaac

SEQ ID NO: 40 - DNA, 53 bp, Artificial Sequence
  Description of Artificial Sequence Synthetic primer
caagcagaagacggcatacgagatcactgtgctaggaacgatgagcctccaac

SEQ ID NO: 41 - DNA, 53 bp, Artificial Sequence
  Description of Artificial Sequence Synthetic primer
caagcagaagacggcatacgagatattggcgctaggaacgatgagcctccaac

SEQ ID NO: 42 - DNA, 53 bp, Artificial Sequence
  Description of Artificial Sequence Synthetic primer
caagcagaagacggcatacgagatgatctggctaggaacgatgagcctccaac

SEQ ID NO: 43 - DNA, 53 bp, Artificial Sequence
  Description of Artificial Sequence Synthetic primer
caagcagaagacggcatacgagattcaagtgctaggaacgatgagcctccaac

SEQ ID NO: 44 - DNA, 53 bp, Artificial Sequence
  Description of Artificial Sequence Synthetic primer
caagcagaagacggcatacgagatctgatcgctaggaacgatgagcctccaac

SEQ ID NO: 45 - DNA, 53 bp, Artificial Sequence
  Description of Artificial Sequence Synthetic primer
caagcagaagacggcatacgagataagctagctaggaacgatgagcctccaac

SEQ ID NO: 46 - DNA, 53 bp, Artificial Sequence
  Description of Artificial Sequence Synthetic primer
caagcagaagacggcatacgagatgtagccgctaggaacgatgagcctccaac

SEQ ID NO: 47 - DNA, 53 bp, Artificial Sequence
  Description of Artificial Sequence Synthetic primer
caagcagaagacggcatacgagattacaaggctaggaacgatgagcctccaac

SEQ ID NO: 48 - DNA, 53 bp, Artificial Sequence
  Description of Artificial Sequence Synthetic primer
caagcagaagacggcatacgagattcatgggctaggaacgatgagcctccaac

SEQ ID NO: 49 - DNA, 53 bp, Artificial Sequence
  Description of Artificial Sequence Synthetic primer
caagcagaagacggcatacgagattgtcttgctaggaacgatgagcctccaac

SEQ ID NO: 50 - DNA, 53 bp, Artificial Sequence
  Description of Artificial Sequence Synthetic primer
caagcagaagacggcatacgagataggaaggctaggaacgatgagcctccaac

SEQ ID NO: 51 - DNA, 53 bp, Artificial Sequence
  Description of Artificial Sequence Synthetic primer
caagcagaagacggcatacgagataaccccgctaggaacgatgagcctccaac

SEQ ID NO: 52 - DNA, 53 bp, Artificial Sequence
  Description of Artificial Sequence Synthetic primer
caagcagaagacggcatacgagatgatgaggctaggaacgatgagcctccaac

SEQ ID NO: 53 - DNA, 53 bp, Artificial Sequence
  Description of Artificial Sequence Synthetic primer
caagcagaagacggcatacgagattgaactgctaggaacgatgagcctccaac

SEQ ID NO: 54 - DNA, 53 bp, Artificial Sequence
  Description of Artificial Sequence Synthetic primer
caagcagaagacggcatacgagattgcgtcgctaggaacgatgagcctccaac

SEQ ID NO: 55 - DNA, 53 bp, Artificial Sequence
  Description of Artificial Sequence Synthetic primer
caagcagaagacggcatacgagatgacagggctaggaacgatgagcctccaac

SEQ ID NO: 56 - DNA, 53 bp, Artificial Sequence
  Description of Artificial Sequence Synthetic primer
caagcagaagacggcatacgagatgggttggctaggaacgatgagcctccaac

SEQ ID NO: 57 - DNA, 53 bp, Artificial Sequence
  Description of Artificial Sequence Synthetic primer
caagcagaagacggcatacgagattccgaggctaggaacgatgagcctccaac

SEQ ID NO: 58 - DNA, 53 bp, Artificial Sequence
  Description of Artificial Sequence Synthetic primer
caagcagaagacggcatacgagattttcgagctaggaacgatgagcctccaac

SEQ ID NO: 59 - DNA, 53 bp, Artificial Sequence
  Description of Artificial Sequence Synthetic primer
caagcagaagacggcatacgagatgcgaatgctaggaacgatgagcctccaac

SEQ ID NO: 60 - DNA, 53 bp, Artificial Sequence
  Description of Artificial Sequence Synthetic primer
caagcagaagacggcatacgagatgcagtagctaggaacgatgagcctccaac

SEQ ID NO: 61 - DNA, 53 bp, Artificial Sequence
  Description of Artificial Sequence Synthetic primer
caagcagaagacggcatacgagattcacgagctaggaacgatgagcctccaac

SEQ ID NO: 62 - DNA, 53 bp, Artificial Sequence
  Description of Artificial Sequence Synthetic primer
caagcagaagacggcatacgagatcgcgtagctaggaacgatgagcctccaac

SEQ ID NO: 63 - DNA, 53 bp, Artificial Sequence
  Description of Artificial Sequence Synthetic primer
caagcagaagacggcatacgagatgcacctgctaggaacgatgagcctccaac

SEQ ID NO: 64 - DNA, 53 bp, Artificial Sequence
  Description of Artificial Sequence Synthetic primer
caagcagaagacggcatacgagatgttcgtgctaggaacgatgagcctccaac

SEQ ID NO: 65 - DNA, 53 bp, Artificial Sequence
  Description of Artificial Sequence Synthetic primer
caagcagaagacggcatacgagatcactaagctaggaacgatgagcctccaac

SEQ ID NO: 66 - DNA, 53 bp, Artificial Sequence
  Description of Artificial Sequence Synthetic primer
caagcagaagacggcatacgagatgtggtggctaggaacgatgagcctccaac

SEQ ID NO: 67 - DNA, 53 bp, Artificial Sequence
  Description of Artificial Sequence Synthetic primer
caagcagaagacggcatacgagatccttgcgctaggaacgatgagcctccaac

SEQ ID NO: 68 - DNA, 53 bp, Artificial Sequence
  Description of Artificial Sequence Synthetic primer
caagcagaagacggcatacgagatgttgcggctaggaacgatgagcctccaac

SEQ ID NO: 69 - DNA, 53 bp, Artificial Sequence
  Description of Artificial Sequence Synthetic primer
caagcagaagacggcatacgagattcagttgctaggaacgatgagcctccaac

SEQ ID NO: 70 - DNA, 53 bp, Artificial Sequence
  Description of Artificial Sequence Synthetic primer
caagcagaagacggcatacgagatcccgatgctaggaacgatgagcctccaac

SEQ ID NO: 71 - DNA, 53 bp, Artificial Sequence
  Description of Artificial Sequence Synthetic primer
caagcagaagacggcatacgagattgcttggctaggaacgatgagcctccaac

SEQ ID NO: 72 - DNA, 53 bp, Artificial Sequence
  Description of Artificial Sequence Synthetic primer
caagcagaagacggcatacgagattgtagcgctaggaacgatgagcctccaac

SEQ ID NO: 73 - DNA, 53 bp, Artificial Sequence
  Description of Artificial Sequence Synthetic primer
caagcagaagacggcatacgagatgcgtgagctaggaacgatgagcctccaac

SEQ ID NO: 74 - DNA, 53 bp, Artificial Sequence
  Description of Artificial Sequence Synthetic primer
caagcagaagacggcatacgagatctctgggctaggaacgatgagcctccaac

SEQ ID NO: 75 - DNA, 53 bp, Artificial Sequence
  Description of Artificial Sequence Synthetic primer
caagcagaagacggcatacgagatccgtcagctaggaacgatgagcctccaac

SEQ ID NO: 76 - DNA, 53 bp, Artificial Sequence
  Description of Artificial Sequence Synthetic primer
caagcagaagacggcatacgagatgttcccgctaggaacgatgagcctccaac

SEQ ID NO: 77 - DNA, 53 bp, Artificial Sequence
  Description of Artificial Sequence Synthetic primer
caagcagaagacggcatacgagatcttttcgctaggaacgatgagcctccaac

SEQ ID NO: 78 - DNA, 53 bp, Artificial Sequence
  Description of Artificial Sequence Synthetic primer
caagcagaagacggcatacgagatggcactgctaggaacgatgagcctccaac

SEQ ID NO: 79 - DNA, 53 bp, Artificial Sequence
  Description of Artificial Sequence Synthetic primer
caagcagaagacggcatacgagatggatgcgctaggaacgatgagcctccaac

SEQ ID NO: 80 - DNA, 53 bp, Artificial Sequence
  Description of Artificial Sequence Synthetic primer
caagcagaagacggcatacgagatcgtagtgctaggaacgatgagcctccaac

SEQ ID NO: 81 - DNA, 53 bp, Artificial Sequence
  Description of Artificial Sequence Synthetic primer
caagcagaagacggcatacgagatgaaatggctaggaacgatgagcctccaac

SEQ ID NO: 82 - DNA, 53 bp, Artificial Sequence
  Description of Artificial Sequence Synthetic primer
caagcagaagacggcatacgagatggagaggctaggaacgatgagcctccaac

SEQ ID NO: 83 - DNA, 53 bp, Artificial Sequence
  Description of Artificial Sequence Synthetic primer
caagcagaagacggcatacgagattaacgtgctaggaacgatgagcctccaac

SEQ ID NO: 84 - DNA, 53 bp, Artificial Sequence
  Description of Artificial Sequence Synthetic primer
caagcagaagacggcatacgagatacacaggctaggaacgatgagcctccaac

SEQ ID NO: 85 - DNA, 53 bp, Artificial Sequence
  Description of Artificial Sequence Synthetic primer
caagcagaagacggcatacgagataaaggtgctaggaacgatgagcctccaac

SEQ ID NO: 86 - DNA, 53 bp, Artificial Sequence
  Description of Artificial Sequence Synthetic primer
caagcagaagacggcatacgagatgcgatagctaggaacgatgagcctccaac

SEQ ID NO: 87 - DNA, 53 bp, Artificial Sequence
  Description of Artificial Sequence Synthetic primer
caagcagaagacggcatacgagatcgtgtcgctaggaacgatgagcctccaac

SEQ ID NO: 88 - DNA, 53 bp, Artificial Sequence
  Description of Artificial Sequence Synthetic primer
caagcagaagacggcatacgagatgtagaagctaggaacgatgagcctccaac

SEQ ID NO: 89 - DNA, 53 bp, Artificial Sequence
  Description of Artificial Sequence Synthetic primer
caagcagaagacggcatacgagatggacgtgctaggaacgatgagcctccaac

SEQ ID NO: 90 - DNA, 53 bp, Artificial Sequence
  Description of Artificial Sequence Synthetic primer
caagcagaagacggcatacgagatagtcgagctaggaacgatgagcctccaac

SEQ ID NO: 91 - DNA, 53 bp, Artificial Sequence
  Description of Artificial Sequence Synthetic primer
caagcagaagacggcatacgagatgtctgagctaggaacgatgagcctccaac

SEQ ID NO: 92 - DNA, 53 bp, Artificial Sequence
  Description of Artificial Sequence Synthetic primer
caagcagaagacggcatacgagatgaaggagctaggaacgatgagcctccaac

SEQ ID NO: 93 - DNA, 53 bp, Artificial Sequence
  Description of Artificial Sequence Synthetic primer
caagcagaagacggcatacgagatatgctggctaggaacgatgagcctccaac

SEQ ID NO: 94 - DNA, 53 bp, Artificial Sequence
  Description of Artificial Sequence Synthetic primer
caagcagaagacggcatacgagattctatcgctaggaacgatgagcctccaac

SEQ ID NO: 95 - DNA, 53 bp, Artificial Sequence
  Description of Artificial Sequence Synthetic primer
caagcagaagacggcatacgagatatctgtgctaggaacgatgagcctccaac

SEQ ID NO: 96 - DNA, 53 bp, Artificial Sequence
  Description of Artificial Sequence Synthetic primer
caagcagaagacggcatacgagatatagaggctaggaacgatgagcctccaac

SEQ ID NO: 97 - DNA, 53 bp, Artificial Sequence
  Description of Artificial Sequence Synthetic primer
caagcagaagacggcatacgagatgctaaagctaggaacgatgagcctccaac

SEQ ID NO: 98 - DNA, 53 bp, Artificial Sequence
  Description of Artificial Sequence Synthetic primer
caagcagaagacggcatacgagataccagggctaggaacgatgagcctccaac

SEQ ID NO: 99 - DNA, 53 bp, Artificial Sequence
  Description of Artificial Sequence Synthetic primer
caagcagaagacggcatacgagatccaactgctaggaacgatgagcctccaac

SEQ ID NO: 100 - DNA, 53 bp, Artificial Sequence
  Description of Artificial Sequence Synthetic primer
caagcagaagacggcatacgagataaggaagctaggaacgatgagcctccaac

SEQ ID NO: 101 - DNA, 53 bp, Artificial Sequence
  Description of Artificial Sequence Synthetic primer
caagcagaagacggcatacgagatcctccagctaggaacgatgagcctccaac

SEQ ID NO: 102 - DNA, 53 bp, Artificial Sequence
  Description of Artificial Sequence Synthetic primer
caagcagaagacggcatacgagatcacgtcgctaggaacgatgagcctccaac

SEQ ID NO: 103 - DNA, 53 bp, Artificial Sequence
  Description of Artificial Sequence Synthetic primer
caagcagaagacggcatacgagatcataacgctaggaacgatgagcctccaac

SEQ ID NO: 104 - DNA, 53 bp, Artificial Sequence
  Description of Artificial Sequence Synthetic primer
caagcagaagacggcatacgagatccatatgctaggaacgatgagcctccaac

SEQ ID NO: 105 - DNA, 53 bp, Artificial Sequence
  Description of Artificial Sequence Synthetic primer
caagcagaagacggcatacgagatgaagtcgctaggaacgatgagcctccaac

SEQ ID NO: 106 - DNA, 53 bp, Artificial Sequence
  Description of Artificial Sequence Synthetic primer
caagcagaagacggcatacgagatcaaagagctaggaacgatgagcctccaac

SEQ ID NO: 107 - DNA, 53 bp, Artificial Sequence
  Description of Artificial Sequence Synthetic primer
caagcagaagacggcatacgagattggcaggctaggaacgatgagcctccaac

SEQ ID NO: 108 - DNA, 53 bp, Artificial Sequence
  Description of Artificial Sequence Synthetic primer
caagcagaagacggcatacgagatgagtccgctaggaacgatgagcctccaac

SEQ ID NO: 109 - DNA, 53 bp, Artificial Sequence
  Description of Artificial Sequence Synthetic primer
caagcagaagacggcatacgagattcgccagctaggaacgatgagcctccaac

SEQ ID NO: 110 - DNA, 53 bp, Artificial Sequence
  Description of Artificial Sequence Synthetic primer
caagcagaagacggcatacgagataagtcggctaggaacgatgagcctccaac

SEQ ID NO: 111 - DNA, 53 bp, Artificial Sequence
  Description of Artificial Sequence Synthetic primer
caagcagaagacggcatacgagataatagggctaggaacgatgagcctccaac

SEQ ID NO: 112 - DNA, 53 bp, Artificial Sequence
  Description of Artificial Sequence Synthetic primer
caagcagaagacggcatacgagatacccgtgctaggaacgatgagcctccaac

SEQ ID NO: 113 - DNA, 53 bp, Artificial Sequence
  Description of Artificial Sequence Synthetic primer
caagcagaagacggcatacgagataacacggctaggaacgatgagcctccaac

SEQ ID NO: 114 - DNA, 53 bp, Artificial Sequence
  Description of Artificial Sequence Synthetic primer
caagcagaagacggcatacgagatgcttgggctaggaacgatgagcctccaac

SEQ ID NO: 115 - DNA, 53 bp, Artificial Sequence
  Description of Artificial Sequence Synthetic primer
caagcagaagacggcatacgagatttaccagctaggaacgatgagcctccaac

SEQ ID NO: 116 - DNA, 53 bp, Artificial Sequence
  Description of Artificial Sequence Synthetic primer
caagcagaagacggcatacgagatccaggtgctaggaacgatgagcctccaac

SEQ ID NO: 117 - DNA, 53 bp, Artificial Sequence
  Description of Artificial Sequence Synthetic primer
caagcagaagacggcatacgagatcgtttggctaggaacgatgagcctccaac

SEQ ID NO: 118 - DNA, 53 bp, Artificial Sequence
  Description of Artificial Sequence Synthetic primer
caagcagaagacggcatacgagatgaccacgctaggaacgatgagcctccaac

SEQ ID NO: 119 - DNA, 53 bp, Artificial Sequence
  Description of Artificial Sequence Synthetic primer
caagcagaagacggcatacgagatacaagagctaggaacgatgagcctccaac

SEQ ID NO: 120 - DNA, 53 bp, Artificial Sequence
  Description of Artificial Sequence Synthetic primer
caagcagaagacggcatacgagataccgcagctaggaacgatgagcctccaac

SEQ ID NO: 121 - DNA, 53 bp, Artificial Sequence
  Description of Artificial Sequence Synthetic primer
caagcagaagacggcatacgagattgggtagctaggaacgatgagcctccaac

SEQ ID NO: 122 - DNA, 53 bp, Artificial Sequence
  Description of Artificial Sequence Synthetic primer
caagcagaagacggcatacgagatattccggctaggaacgatgagcctccaac

SEQ ID NO: 123 - DNA, 53 bp, Artificial Sequence
  Description of Artificial Sequence Synthetic primer
caagcagaagacggcatacgagatgaatgtgctaggaacgatgagcctccaac

SEQ ID NO: 124 - DNA, 53 bp, Artificial Sequence
  Description of Artificial Sequence Synthetic primer
caagcagaagacggcatacgagatgctgatgctaggaacgatgagcctccaac

SEQ ID NO: 125 - DNA, 53 bp, Artificial Sequence
  Description of Artificial Sequence Synthetic primer
caagcagaagacggcatacgagatagtgctgctaggaacgatgagcctccaac

SEQ ID NO: 126 - DNA, 53 bp, Artificial Sequence
  Description of Artificial Sequence Synthetic primer
caagcagaagacggcatacgagatcagggagctaggaacgatgagcctccaac

SEQ ID NO: 127 - DNA, 53 bp, Artificial Sequence
  Description of Artificial Sequence Synthetic primer
caagcagaagacggcatacgagatcatgcggctaggaacgatgagcctccaac

SEQ ID NO: 128 - DNA, 53 bp, Artificial Sequence
  Description of Artificial Sequence Synthetic primer
caagcagaagacggcatacgagattgcctagctaggaacgatgagcctccaac

SEQ ID NO: 129 - DNA, 53 bp, Artificial Sequence
  Description of Artificial Sequence Synthetic primer
caagcagaagacggcatacgagatctatacgctaggaacgatgagcctccaac

SEQ ID NO: 130 - DNA, 53 bp, Artificial Sequence
  Description of Artificial Sequence Synthetic primer
caagcagaagacggcatacgagatccgagtgctaggaacgatgagcctccaac

SEQ ID NO: 131 - DNA, 53 bp, Artificial Sequence
  Description of Artificial Sequence Synthetic primer
caagcagaagacggcatacgagatacctgcgctaggaacgatgagcctccaac

SEQ ID NO: 132 - DNA, 53 bp, Artificial Sequence
  Description of Artificial Sequence Synthetic primer
caagcagaagacggcatacgagatcaggacgctaggaacgatgagcctccaac

SEQ ID NO: 133 - DNA, 53 bp, Artificial Sequence
  Description of Artificial Sequence Synthetic primer
caagcagaagacggcatacgagataagatggctaggaacgatgagcctccaac

SEQ ID NO: 134 - DNA, 53 bp, Artificial Sequence
  Description of Artificial Sequence Synthetic primer
caagcagaagacggcatacgagatggcttcgctaggaacgatgagcctccaac

SEQ ID NO: 135 - DNA, 53 bp, Artificial Sequence
  Description of Artificial Sequence Synthetic primer
caagcagaagacggcatacgagatgtgcttgctaggaacgatgagcctccaac

SEQ ID NO: 136 - DNA, 53 bp, Artificial Sequence
  Description of Artificial Sequence Synthetic primer
caagcagaagacggcatacgagattgcgcagctaggaacgatgagcctccaac

SEQ ID NO: 137 - DNA, 53 bp, Artificial Sequence
  Description of Artificial Sequence Synthetic primer
caagcagaagacggcatacgagatactagcgctaggaacgatgagcctccaac

SEQ ID NO: 138 - DNA, 53 bp, Artificial Sequence
  Description of Artificial Sequence Synthetic primer
caagcagaagacggcatacgagattcgaaggctaggaacgatgagcctccaac

SEQ ID NO: 139 - DNA, 53 bp, Artificial Sequence
  Description of Artificial Sequence Synthetic primer
caagcagaagacggcatacgagatagactagctaggaacgatgagcctccaac

SEQ ID NO: 140 - DNA, 53 bp, Artificial Sequence
  Description of Artificial Sequence Synthetic primer
caagcagaagacggcatacgagatcgggttgctaggaacgatgagcctccaac

SEQ ID NO: 141 - DNA, 53 bp, Artificial Sequence
  Description of Artificial Sequence Synthetic primer
caagcagaagacggcatacgagattgactggctaggaacgatgagcctccaac

SEQ ID NO: 142 - DNA, 53 bp, Artificial Sequence
  Description of Artificial Sequence Synthetic primer
caagcagaagacggcatacgagatttgtgtgctaggaacgatgagcctccaac

SEQ ID NO: 143 - DNA, 53 bp, Artificial Sequence
  Description of Artificial Sequence Synthetic primer
caagcagaagacggcatacgagattcgctggctaggaacgatgagcctccaac

SEQ ID NO: 144 - DNA, 53 bp, Artificial Sequence
  Description of Artificial Sequence Synthetic primer
caagcagaagacggcatacgagatgatacagctaggaacgatgagcctccaac

SEQ ID NO: 145 - DNA, 53 bp, Artificial Sequence
  Description of Artificial Sequence Synthetic primer
caagcagaagacggcatacgagattccttagctaggaacgatgagcctccaac

SEQ ID NO: 146 - DNA, 53 bp, Artificial Sequence
  Description of Artificial Sequence Synthetic primer
caagcagaagacggcatacgagatcgatttgctaggaacgatgagcctccaac

SEQ ID NO: 147 - DNA, 53 bp, Artificial Sequence
  Description of Artificial Sequence Synthetic primer
caagcagaagacggcatacgagatttacgggctaggaacgatgagcctccaac

SEQ ID NO: 148 - DNA, 53 bp, Artificial Sequence
  Description of Artificial Sequence Synthetic primer
caagcagaagacggcatacgagattgtgacgctaggaacgatgagcctccaac

SEQ ID NO: 149 - DNA, 53 bp, Artificial Sequence
  Description of Artificial Sequence Synthetic primer
caagcagaagacggcatacgagatttggaagctaggaacgatgagcctccaac

SEQ ID NO: 150 - DNA, 53 bp, Artificial Sequence
  Description of Artificial Sequence Synthetic primer
caagcagaagacggcatacgagataccatagctaggaacgatgagcctccaac

SEQ ID NO: 151 - DNA, 53 bp, Artificial Sequence
  Description of Artificial Sequence Synthetic primer
caagcagaagacggcatacgagatgtcgaggctaggaacgatgagcctccaac

SEQ ID NO: 152 - DNA, 53 bp, Artificial Sequence
  Description of Artificial Sequence Synthetic primer
caagcagaagacggcatacgagatactgccgctaggaacgatgagcctccaac

SEQ ID NO: 153 - DNA, 53 bp, Artificial Sequence
  Description of Artificial Sequence Synthetic primer
caagcagaagacggcatacgagattcgggtgctaggaacgatgagcctccaac

SEQ ID NO: 154 - DNA, 53 bp, Artificial Sequence
  Description of Artificial Sequence Synthetic primer
caagcagaagacggcatacgagatggcatggctaggaacgatgagcctccaac

SEQ ID NO: 155 - DNA, 53 bp, Artificial Sequence
  Description of Artificial Sequence Synthetic primer
caagcagaagacggcatacgagatgtttctgctaggaacgatgagcctccaac

SEQ ID NO: 156 - DNA, 53 bp, Artificial Sequence
  Description of Artificial Sequence Synthetic primer
caagcagaagacggcatacgagataccaatgctaggaacgatgagcctccaac

SEQ ID NO: 157 - DNA, 53 bp, Artificial Sequence
  Description of Artificial Sequence Synthetic primer
caagcagaagacggcatacgagatatgcatgctaggaacgatgagcctccaac

SEQ ID NO: 158 - DNA, 53 bp, Artificial Sequence
  Description of Artificial Sequence Synthetic primer
caagcagaagacggcatacgagattacggcgctaggaacgatgagcctccaac

SEQ ID NO: 159 - DNA, 53 bp, Artificial Sequence
  Description of Artificial Sequence Synthetic primer
caagcagaagacggcatacgagatagtcccgctaggaacgatgagcctccaac

SEQ ID NO: 160 - DNA, 53 bp, Artificial Sequence
  Description of Artificial Sequence Synthetic primer
caagcagaagacggcatacgagatctgcaggctaggaacgatgagcctccaac

SEQ ID NO: 161 - DNA, 53 bp, Artificial Sequence
  Description of Artificial Sequence Synthetic primer
caagcagaagacggcatacgagatctgttggctaggaacgatgagcctccaac

SEQ ID NO: 162 - DNA, 53 bp, Artificial Sequence
  Description of Artificial Sequence Synthetic primer
caagcagaagacggcatacgagatcggacagctaggaacgatgagcctccaac

SEQ ID NO: 163 - DNA, 53 bp, Artificial Sequence
  Description of Artificial Sequence Synthetic primer
caagcagaagacggcatacgagattaagcggctaggaacgatgagcctccaac

SEQ ID NO: 164 - DNA, 53 bp, Artificial Sequence
  Description of Artificial Sequence Synthetic primer
caagcagaagacggcatacgagatgagagtgctaggaacgatgagcctccaac

SEQ ID NO: 165 - DNA, 53 bp, Artificial Sequence
  Description of Artificial Sequence Synthetic primer
caagcagaagacggcatacgagattacccggctaggaacgatgagcctccaac

SEQ ID NO: 166 - DNA, 53 bp, Artificial Sequence
  Description of Artificial Sequence Synthetic primer
caagcagaagacggcatacgagatttcgtagctaggaacgatgagcctccaac

SEQ ID NO: 167 - DNA, 53 bp, Artificial Sequence
  Description of Artificial Sequence Synthetic primer
caagcagaagacggcatacgagataaagtggctaggaacgatgagcctccaac

SEQ ID NO: 168 - DNA, 53 bp, Artificial Sequence
  Description of Artificial Sequence Synthetic primer
caagcagaagacggcatacgagattttggtgctaggaacgatgagcctccaac

SEQ ID NO: 169 - DNA, 53 bp, Artificial Sequence
  Description of Artificial Sequence Synthetic primer
caagcagaagacggcatacgagatgtccctgctaggaacgatgagcctccaac

SEQ ID NO: 170 - DNA, 53 bp, Artificial Sequence
  Description of Artificial Sequence Synthetic primer
caagcagaagacggcatacgagattagcttgctaggaacgatgagcctccaac

SEQ ID NO: 171 - DNA, 53 bp, Artificial Sequence
  Description of Artificial Sequence Synthetic primer
caagcagaagacggcatacgagatgcactggctaggaacgatgagcctccaac

SEQ ID NO: 172 - DNA, 53 bp, Artificial Sequence
  Description of Artificial Sequence Synthetic primer
caagcagaagacggcatacgagatactatggctaggaacgatgagcctccaac

SEQ ID NO: 173 - DNA, 53 bp, Artificial Sequence
  Description of Artificial Sequence Synthetic primer
caagcagaagacggcatacgagatgaacttgctaggaacgatgagcctccaac

SEQ ID NO: 174 - DNA, 53 bp, Artificial Sequence
  Description of Artificial Sequence Synthetic primer
caagcagaagacggcatacgagattttgaggctaggaacgatgagcctccaac

SEQ ID NO: 175 - DNA, 53 bp, Artificial Sequence
  Description of Artificial Sequence Synthetic primer
caagcagaagacggcatacgagataggcctgctaggaacgatgagcctccaac

SEQ ID NO: 176 - DNA, 53 bp, Artificial Sequence
  Description of Artificial Sequence Synthetic primer
caagcagaagacggcatacgagatacacgcgctaggaacgatgagcctccaac

SEQ ID NO: 177 - DNA, 53 bp, Artificial Sequence
  Description of Artificial Sequence Synthetic primer
caagcagaagacggcatacgagatttgtcggctaggaacgatgagcctccaac

SEQ ID NO: 178 - DNA, 53 bp, Artificial Sequence
  Description of Artificial Sequence Synthetic primer
caagcagaagacggcatacgagattctcacgctaggaacgatgagcctccaac

SEQ ID NO: 179 - DNA, 53 bp, Artificial Sequence
  Description of Artificial Sequence Synthetic primer
caagcagaagacggcatacgagattagaccgctaggaacgatgagcctccaac

SEQ ID NO: 180 - DNA, 53 bp, Artificial Sequence
  Description of Artificial Sequence Synthetic primer
caagcagaagacggcatacgagatcctaaggctaggaacgatgagcctccaac

SEQ ID NO: 181 - DNA, 53 bp, Artificial Sequence
  Description of Artificial Sequence Synthetic primer
caagcagaagacggcatacgagatgtatcggctaggaacgatgagcctccaac

SEQ ID NO: 182 - DNA, 53 bp, Artificial Sequence
  Description of Artificial Sequence Synthetic primer
caagcagaagacggcatacgagattccagagctaggaacgatgagcctccaac

SEQ ID NO: 183 - DNA, 53 bp, Artificial Sequence
  Description of Artificial Sequence Synthetic primer
caagcagaagacggcatacgagataggtgagctaggaacgatgagcctccaac

SEQ ID NO: 184 - DNA, 53 bp, Artificial Sequence
  Description of Artificial Sequence Synthetic primer
caagcagaagacggcatacgagatcccatcgctaggaacgatgagcctccaac

SEQ ID NO: 185 - DNA, 53 bp, Artificial Sequence
  Description of Artificial Sequence Synthetic primer
caagcagaagacggcatacgagatttgcacgctaggaacgatgagcctccaac

SEQ ID NO: 186 - DNA, 53 bp, Artificial Sequence
  Description of Artificial Sequence Synthetic primer
caagcagaagacggcatacgagataactcagctaggaacgatgagcctccaac

SEQ ID NO: 187 - DNA, 53 bp, Artificial Sequence
  Description of Artificial Sequence Synthetic primer
caagcagaagacggcatacgagatcgtatagctaggaacgatgagcctccaac

SEQ ID NO: 188 - DNA, 53 bp, Artificial Sequence
  Description of Artificial Sequence Synthetic primer
caagcagaagacggcatacgagatagcgaagctaggaacgatgagcctccaac

SEQ ID NO: 189 - DNA, 53 bp, Artificial Sequence
  Description of Artificial Sequence Synthetic primer
caagcagaagacggcatacgagatacggctgctaggaacgatgagcctccaac

SEQ ID NO: 190 - DNA, 53 bp, Artificial Sequence
  Description of Artificial Sequence Synthetic primer
caagcagaagacggcatacgagatagtgaggctaggaacgatgagcctccaac

SEQ ID NO: 191 - DNA, 53 bp, Artificial Sequence
  Description of Artificial Sequence Synthetic primer
caagcagaagacggcatacgagattttctcgctaggaacgatgagcctccaac

SEQ ID NO: 192 - DNA, 53 bp, Artificial Sequence
  Description of Artificial Sequence Synthetic primer
caagcagaagacggcatacgagatgctctagctaggaacgatgagcctccaac

SEQ ID NO: 193 - DNA, 53 bp, Artificial Sequence
  Description of Artificial Sequence Synthetic primer
caagcagaagacggcatacgagatacttgagctaggaacgatgagcctccaac

SEQ ID NO: 194 - DNA, 53 bp, Artificial Sequence
  Description of Artificial Sequence Synthetic primer
caagcagaagacggcatacgagatcggttcgctaggaacgatgagcctccaac

SEQ ID NO: 195 - DNA, 53 bp, Artificial Sequence
  Description of Artificial Sequence Synthetic primer
caagcagaagacggcatacgagatcatcatgctaggaacgatgagcctccaac

SEQ ID NO: 196 - DNA, 53 bp, Artificial Sequence
  Description of Artificial Sequence Synthetic primer
caagcagaagacggcatacgagatcaaacggctaggaacgatgagcctccaac

SEQ ID NO: 197 - DNA, 53 bp, Artificial Sequence
  Description of Artificial Sequence Synthetic primer
caagcagaagacggcatacgagatctatgtgctaggaacgatgagcctccaac

SEQ ID NO: 198 - DNA, 53 bp, Artificial Sequence
  Description of Artificial Sequence Synthetic primer
caagcagaagacggcatacgagatagcgttgctaggaacgatgagcctccaac

SEQ ID NO: 199 - DNA, 53 bp, Artificial Sequence
  Description of Artificial Sequence Synthetic primer
caagcagaagacggcatacgagataagactgctaggaacgatgagcctccaac

SEQ ID NO: 200 - DNA, 53 bp, Artificial Sequence
  Description of Artificial Sequence Synthetic primer
caagcagaagacggcatacgagatcgataagctaggaacgatgagcctccaac

SEQ ID NO: 201 - DNA, 53 bp, Artificial Sequence
  Description of Artificial Sequence Synthetic primer
caagcagaagacggcatacgagatcggctagctaggaacgatgagcctccaac

SEQ ID NO: 202 - DNA, 53 bp, Artificial Sequence
  Description of Artificial Sequence Synthetic primer
caagcagaagacggcatacgagattatacggctaggaacgatgagcctccaac

SEQ ID NO: 203 - DNA, 53 bp, Artificial Sequence
  Description of Artificial Sequence Synthetic primer
caagcagaagacggcatacgagatgaaaccgctaggaacgatgagcctccaac

SEQ ID NO: 204 - DNA, 53 bp, Artificial Sequence
  Description of Artificial Sequence Synthetic primer
caagcagaagacggcatacgagatgaaccggctaggaacgatgagcctccaac

SEQ ID NO: 205 - DNA, 53 bp, Artificial Sequence
  Description of Artificial Sequence Synthetic primer
caagcagaagacggcatacgagatttaggcgctaggaacgatgagcctccaac

SEQ ID NO: 206 - DNA, 53 bp, Artificial Sequence
  Description of Artificial Sequence Synthetic primer
caagcagaagacggcatacgagatgcctttgctaggaacgatgagcctccaac

SEQ ID NO: 207 - DNA, 53 bp, Artificial Sequence
  Description of Artificial Sequence Synthetic primer
caagcagaagacggcatacgagatgttggagctaggaacgatgagcctccaac

SEQ ID NO: 208 - DNA, 53 bp, Artificial Sequence
  Description of Artificial Sequence Synthetic primer
caagcagaagacggcatacgagatgtacgcgctaggaacgatgagcctccaac

SEQ ID NO: 209 - DNA, 53 bp, Artificial Sequence
  Description of Artificial Sequence Synthetic primer
caagcagaagacggcatacgagattggaacgctaggaacgatgagcctccaac

SEQ ID NO: 210 - DNA, 53 bp, Artificial Sequence
  Description of Artificial Sequence Synthetic primer
caagcagaagacggcatacgagataacagagctaggaacgatgagcctccaac

SEQ ID NO: 211 - DNA, 53 bp, Artificial Sequence
  Description of Artificial Sequence Synthetic primer
caagcagaagacggcatacgagataagcacgctaggaacgatgagcctccaac

SEQ ID NO: 212 - DNA, 53 bp, Artificial Sequence
  Description of Artificial Sequence Synthetic primer
caagcagaagacggcatacgagatatggtagctaggaacgatgagcctccaac

SEQ ID NO: 213 - DNA, 53 bp, Artificial Sequence
  Description of Artificial Sequence Synthetic primer
caagcagaagacggcatacgagattcgttcgctaggaacgatgagcctccaac

SEQ ID NO: 214 - DNA, 53 bp, Artificial Sequence
  Description of Artificial Sequence Synthetic primer
caagcagaagacggcatacgagatcttctagctaggaacgatgagcctccaac

SEQ ID NO: 215 - DNA, 53 bp, Artificial Sequence
  Description of Artificial Sequence Synthetic primer
caagcagaagacggcatacgagattgggatgctaggaacgatgagcctccaac

SEQ ID NO: 216 - DNA, 53 bp, Artificial Sequence
  Description of Artificial Sequence Synthetic primer
caagcagaagacggcatacgagatatcgccgctaggaacgatgagcctccaac

SEQ ID NO: 217 - DNA, 53 bp, Artificial Sequence
  Description of Artificial Sequence Synthetic primer
caagcagaagacggcatacgagatagttgggctaggaacgatgagcctccaac

SEQ ID NO: 218 - DNA, 53 bp, Artificial Sequence
  Description of Artificial Sequence Synthetic primer
caagcagaagacggcatacgagatagctctgctaggaacgatgagcctccaac

SEQ ID NO: 219 - DNA, 53 bp, Artificial Sequence
  Description of Artificial Sequence Synthetic primer
caagcagaagacggcatacgagatgacggtgctaggaacgatgagcctccaac

SEQ ID NO: 220 - DNA, 53 bp, Artificial Sequence
  Description of Artificial Sequence Synthetic primer
caagcagaagacggcatacgagatctcagcgctaggaacgatgagcctccaac

SEQ ID NO: 221 - DNA, 53 bp, Artificial Sequence
  Description of Artificial Sequence Synthetic primer
caagcagaagacggcatacgagatcttagggctaggaacgatgagcctccaac

SEQ ID NO: 222 - DNA, 53 bp, Artificial Sequence
  Description of Artificial Sequence Synthetic primer
caagcagaagacggcatacgagatcgacaggctaggaacgatgagcctccaac

SEQ ID NO: 223 - DNA, 53 bp, Artificial Sequence
  Description of Artificial Sequence Synthetic primer
caagcagaagacggcatacgagatacatgtgctaggaacgatgagcctccaac

SEQ ID NO: 224 - DNA, 53 bp, Artificial Sequence
  Description of Artificial Sequence Synthetic primer
caagcagaagacggcatacgagatatacgagctaggaacgatgagcctccaac

SEQ ID NO: 225 - DNA, 53 bp, Artificial Sequence
  Description of Artificial Sequence Synthetic primer
caagcagaagacggcatacgagatgagcatgctaggaacgatgagcctccaac

SEQ ID NO: 226 - DNA, 53 bp, Artificial Sequence
  Description of Artificial Sequence Synthetic primer
caagcagaagacggcatacgagatatcctagctaggaacgatgagcctccaac

SEQ ID NO: 227 - DNA, 53 bp, Artificial Sequence
  Description of Artificial Sequence Synthetic primer
caagcagaagacggcatacgagatacgtaagctaggaacgatgagcctccaac

SEQ ID NO: 228 - DNA, 53 bp, Artificial Sequence
  Description of Artificial Sequence Synthetic primer
caagcagaagacggcatacgagatggaaacgctaggaacgatgagcctccaac

SEQ ID NO: 229 - DNA, 53 bp, Artificial Sequence
  Description of Artificial Sequence Synthetic primer
caagcagaagacggcatacgagatagcaacgctaggaacgatgagcctccaac

SEQ ID NO: 230 - DNA, 53 bp, Artificial Sequence
  Description of Artificial Sequence Synthetic primer
caagcagaagacggcatacgagatctagtggctaggaacgatgagcctccaac

SEQ ID NO: 231 - DNA, 53 bp, Artificial Sequence
  Description of Artificial Sequence Synthetic primer
caagcagaagacggcatacgagatccgcttgctaggaacgatgagcctccaac

SEQ ID NO: 232 - DNA, 53 bp, Artificial Sequence
  Description of Artificial Sequence Synthetic primer
caagcagaagacggcatacgagatccagcagctaggaacgatgagcctccaac

SEQ ID NO: 233 - DNA, 53 bp, Artificial Sequence
  Description of Artificial Sequence Synthetic primer
caagcagaagacggcatacgagatatactcgctaggaacgatgagcctccaac

SEQ ID NO: 234 - DNA, 53 bp, Artificial Sequence
  Description of Artificial Sequence Synthetic primer
caagcagaagacggcatacgagatggtgaagctaggaacgatgagcctccaac

SEQ ID NO: 235 - DNA, 53 bp, Artificial Sequence
  Description of Artificial Sequence Synthetic primer
caagcagaagacggcatacgagatctctatgctaggaacgatgagcctccaac

SEQ ID NO: 236 - DNA, 53 bp, Artificial Sequence
  Description of Artificial Sequence Synthetic primer
caagcagaagacggcatacgagatgacatagctaggaacgatgagcctccaac

SEQ ID NO: 237 - DNA, 53 bp, Artificial Sequence
  Description of Artificial Sequence Synthetic primer
caagcagaagacggcatacgagatggatctgctaggaacgatgagcctccaac

SEQ ID NO: 238 - DNA, 53 bp, Artificial Sequence
  Description of Artificial Sequence Synthetic primer
caagcagaagacggcatacgagataacgaggctaggaacgatgagcctccaac

SEQ ID NO: 239 - DNA, 53 bp, Artificial Sequence
  Description of Artificial Sequence Synthetic primer
caagcagaagacggcatacgagatcacctggctaggaacgatgagcctccaac

SEQ ID NO: 240 - DNA, 53 bp, Artificial Sequence
  Description of Artificial Sequence Synthetic primer
caagcagaagacggcatacgagatcatgaagctaggaacgatgagcctccaac

SEQ ID NO: 241 - DNA, 53 bp, Artificial Sequence
  Description of Artificial Sequence Synthetic primer
caagcagaagacggcatacgagatcgtggagctaggaacgatgagcctccaac

SEQ ID NO: 242 - DNA, 53 bp, Artificial Sequence
  Description of Artificial Sequence Synthetic primer
caagcagaagacggcatacgagatagaagggctaggaacgatgagcctccaac

SEQ ID NO: 243 - DNA, 53 bp, Artificial Sequence
  Description of Artificial Sequence Synthetic primer
caagcagaagacggcatacgagatatccaggctaggaacgatgagcctccaac

SEQ ID NO: 244 - DNA, 53 bp, Artificial Sequence
  Description of Artificial Sequence Synthetic primer
caagcagaagacggcatacgagatttcctggctaggaacgatgagcctccaac

SEQ ID NO: 245 - DNA, 53 bp, Artificial Sequence
  Description of Artificial Sequence Synthetic primer
caagcagaagacggcatacgagatcaacaagctaggaacgatgagcctccaac

SEQ ID NO: 246 - DNA, 53 bp, Artificial Sequence
  Description of Artificial Sequence Synthetic primer
caagcagaagacggcatacgagatcctgttgctaggaacgatgagcctccaac

SEQ ID NO: 247 - DNA, 53 bp, Artificial Sequence
  Description of Artificial Sequence Synthetic primer
caagcagaagacggcatacgagatctcgttgctaggaacgatgagcctccaac

SEQ ID NO: 248 - DNA, 53 bp, Artificial Sequence
  Description of Artificial Sequence Synthetic primer
caagcagaagacggcatacgagatctgagagctaggaacgatgagcctccaac

SEQ ID NO: 249 - DNA, 53 bp, Artificial Sequence
  Description of Artificial Sequence Synthetic primer
caagcagaagacggcatacgagatcgctcagctaggaacgatgagcctccaac

SEQ ID NO: 250 - DNA, 53 bp, Artificial Sequence
  Description of Artificial Sequence Synthetic primer
caagcagaagacggcatacgagattaccaagctaggaacgatgagcctccaac

SEQ ID NO: 251 - DNA, 53 bp, Artificial Sequence
  Description of Artificial Sequence Synthetic primer
caagcagaagacggcatacgagatcgcaaggctaggaacgatgagcctccaac

SEQ ID NO: 252 - DNA, 53 bp, Artificial Sequence
  Description of Artificial Sequence Synthetic primer
caagcagaagacggcatacgagattactgagctaggaacgatgagcctccaac

SEQ ID NO: 253 - DNA, 53 bp, Artificial Sequence
  Description of Artificial Sequence Synthetic primer
caagcagaagacggcatacgagattccactgctaggaacgatgagcctccaac

SEQ ID NO: 254 - DNA, 53 bp, Artificial Sequence
  Description of Artificial Sequence Synthetic primer
caagcagaagacggcatacgagatatcgtggctaggaacgatgagcctccaac

SEQ ID NO: 255 - DNA, 53 bp, Artificial Sequence
  Description of Artificial Sequence Synthetic primer
caagcagaagacggcatacgagattaggcagctaggaacgatgagcctccaac

SEQ ID NO: 256 - DNA, 53 bp, Artificial Sequence
  Description of Artificial Sequence Synthetic primer
caagcagaagacggcatacgagatcaagaggctaggaacgatgagcctccaac

SEQ ID NO: 257 - DNA, 53 bp, Artificial Sequence
  Description of Artificial Sequence Synthetic primer
caagcagaagacggcatacgagatacgacagctaggaacgatgagcctccaac

SEQ ID NO: 258 - DNA, 53 bp, Artificial Sequence
  Description of Artificial Sequence Synthetic primer
caagcagaagacggcatacgagatggttccgctaggaacgatgagcctccaac

SEQ ID NO: 259 - DNA, 53 bp, Artificial Sequence
  Description of Artificial Sequence Synthetic primer
caagcagaagacggcatacgagattcttaggctaggaacgatgagcctccaac

SEQ ID NO: 260 - DNA, 53 bp, Artificial Sequence
  Description of Artificial Sequence Synthetic primer
caagcagaagacggcatacgagatagaggagctaggaacgatgagcctccaac

SEQ ID NO: 261 - DNA, 53 bp, Artificial Sequence
  Description of Artificial Sequence Synthetic primer
caagcagaagacggcatacgagataaagcagctaggaacgatgagcctccaac

SEQ ID NO: 262 - DNA, 53 bp, Artificial Sequence
  Description of Artificial Sequence Synthetic primer
caagcagaagacggcatacgagatagtcatgctaggaacgatgagcctccaac

SEQ ID NO: 263 - DNA, 53 bp, Artificial Sequence
  Description of Artificial Sequence Synthetic primer
caagcagaagacggcatacgagattgcagtgctaggaacgatgagcctccaac

SEQ ID NO: 264 - DNA, 53 bp, Artificial Sequence
  Description of Artificial Sequence Synthetic primer
caagcagaagacggcatacgagattgttctgctaggaacgatgagcctccaac

SEQ ID NO: 265 - DNA, 53 bp, Artificial Sequence
  Description of Artificial Sequence Synthetic primer
caagcagaagacggcatacgagattatcgcgctaggaacgatgagcctccaac

SEQ ID NO: 266 - DNA, 53 bp, Artificial Sequence
  Description of Artificial Sequence Synthetic primer
caagcagaagacggcatacgagatgcatcagctaggaacgatgagcctccaac

SEQ ID NO: 267 - DNA, 53 bp, Artificial Sequence
  Description of Artificial Sequence Synthetic primer
caagcagaagacggcatacgagatctccgtgctaggaacgatgagcctccaac

SEQ ID NO: 268 - DNA, 53 bp, Artificial Sequence
  Description of Artificial Sequence Synthetic primer
caagcagaagacggcatacgagattaagacgctaggaacgatgagcctccaac

SEQ ID NO: 269 - DNA, 53 bp, Artificial Sequence
  Description of Artificial Sequence Synthetic primer
caagcagaagacggcatacgagatgaggctgctaggaacgatgagcctccaac

SEQ ID NO: 270 - DNA, 53 bp, Artificial Sequence
  Description of Artificial Sequence Synthetic primer
caagcagaagacggcatacgagattgattcgctaggaacgatgagcctccaac

SEQ ID NO: 271 - DNA, 53 bp, Artificial Sequence
  Description of Artificial Sequence Synthetic primer
caagcagaagacggcatacgagatgtccaagctaggaacgatgagcctccaac

SEQ ID NO: 272 - DNA, 53 bp, Artificial Sequence
  Description of Artificial Sequence Synthetic primer
caagcagaagacggcatacgagatactctcgctaggaacgatgagcctccaac

SEQ ID NO: 273 - DNA, 53 bp, Artificial Sequence
  Description of Artificial Sequence Synthetic primer
caagcagaagacggcatacgagattcaacggctaggaacgatgagcctccaac

SEQ ID NO: 274 - DNA, 53 bp, Artificial Sequence
  Description of Artificial Sequence Synthetic primer
caagcagaagacggcatacgagatgggtacgctaggaacgatgagcctccaac

SEQ ID NO: 275 - DNA, 53 bp, Artificial Sequence
  Description of Artificial Sequence Synthetic primer
caagcagaagacggcatacgagatacgattgctaggaacgatgagcctccaac

SEQ ID NO: 276 - DNA, 53 bp, Artificial Sequence
  Description of Artificial Sequence Synthetic primer
caagcagaagacggcatacgagataacttggctaggaacgatgagcctccaac

SEQ ID NO: 277 - DNA, 53 bp, Artificial Sequence
  Description of Artificial Sequence Synthetic primer
caagcagaagacggcatacgagataacgtagctaggaacgatgagcctccaac

SEQ ID NO: 278 - DNA, 53 bp, Artificial Sequence
  Description of Artificial Sequence Synthetic primer
caagcagaagacggcatacgagatacccacgctaggaacgatgagcctccaac

SEQ ID NO: 279 - DNA, 53 bp, Artificial Sequence
  Description of Artificial Sequence Synthetic primer
caagcagaagacggcatacgagatcggtctgctaggaacgatgagcctccaac

SEQ ID NO: 280 - DNA, 53 bp, Artificial Sequence
  Description of Artificial Sequence Synthetic primer
caagcagaagacggcatacgagatttctaggctaggaacgatgagcctccaac

SEQ ID NO: 281 - DNA, 53 bp, Artificial Sequence
  Description of Artificial Sequence Synthetic primer
caagcagaagacggcatacgagatggtgtggctaggaacgatgagcctccaac

SEQ ID NO: 282 - DNA, 53 bp, Artificial Sequence
  Description of Artificial Sequence Synthetic primer
caagcagaagacggcatacgagataccaccgctaggaacgatgagcctccaac

SEQ ID NO: 283 - DNA, 53 bp, Artificial Sequence
  Description of Artificial Sequence Synthetic primer
caagcagaagacggcatacgagatgactgcgctaggaacgatgagcctccaac

SEQ ID NO: 284 - DNA, 53 bp, Artificial Sequence
  Description of Artificial Sequence Synthetic primer
caagcagaagacggcatacgagatctacttgctaggaacgatgagcctccaac

SEQ ID NO: 285 - DNA, 53 bp, Artificial Sequence
  Description of Artificial Sequence Synthetic primer
caagcagaagacggcatacgagattagcgggctaggaacgatgagcctccaac

SEQ ID NO: 286 - DNA, 53 bp, Artificial Sequence
  Description of Artificial Sequence Synthetic primer
caagcagaagacggcatacgagattgtgcggctaggaacgatgagcctccaac

SEQ ID NO: 287 - DNA, 53 bp, Artificial Sequence
  Description of Artificial Sequence Synthetic primer
caagcagaagacggcatacgagatctggctgctaggaacgatgagcctccaac

SEQ ID NO: 288 - DNA, 53 bp, Artificial Sequence
  Description of Artificial Sequence Synthetic primer
caagcagaagacggcatacgagatcgagacgctaggaacgatgagcctccaac

SEQ ID NO: 289 - DNA, 53 bp, Artificial Sequence
  Description of Artificial Sequence Synthetic primer
caagcagaagacggcatacgagatgggattgctaggaacgatgagcctccaac

SEQ ID NO: 290 - DNA, 53 bp, Artificial Sequence
  Description of Artificial Sequence Synthetic primer
caagcagaagacggcatacgagattactccgctaggaacgatgagcctccaac

SEQ ID NO: 291 - DNA, 53 bp, Artificial Sequence
  Description of Artificial Sequence Synthetic primer
caagcagaagacggcatacgagatgtggcagctaggaacgatgagcctccaac

SEQ ID NO: 292 - DNA, 53 bp, Artificial Sequence
  Description of Artificial Sequence Synthetic primer
caagcagaagacggcatacgagatgtcgtcgctaggaacgatgagcctccaac

SEQ ID NO: 293 - DNA, 53 bp, Artificial Sequence
  Description of Artificial Sequence Synthetic primer
caagcagaagacggcatacgagatgcattcgctaggaacgatgagcctccaac

SEQ ID NO: 294 - DNA, 53 bp, Artificial Sequence
  Description of Artificial Sequence Synthetic primer
caagcagaagacggcatacgagatgagcgagctaggaacgatgagcctccaac

SEQ ID NO: 295 - DNA, 53 bp, Artificial Sequence
  Description of Artificial Sequence Synthetic primer
caagcagaagacggcatacgagatagacacgctaggaacgatgagcctccaac

SEQ ID NO: 296 - DNA, 53 bp, Artificial Sequence
  Description of Artificial Sequence Synthetic primer
caagcagaagacggcatacgagattctggggctaggaacgatgagcctccaac

SEQ ID NO: 297 - DNA, 53 bp, Artificial Sequence
  Description of Artificial Sequence Synthetic primer
caagcagaagacggcatacgagatgccagtgctaggaacgatgagcctccaac

SEQ ID NO: 298 - DNA, 53 bp, Artificial Sequence
  Description of Artificial Sequence Synthetic primer
caagcagaagacggcatacgagatccagtcgctaggaacgatgagcctccaac

SEQ ID NO: 299 - DNA, 53 bp, Artificial Sequence
  Description of Artificial Sequence Synthetic primer
caagcagaagacggcatacgagatttggccgctaggaacgatgagcctccaac

SEQ ID NO: 300 - DNA, 53 bp, Artificial Sequence
  Description of Artificial Sequence Synthetic primer
caagcagaagacggcatacgagatgtaacagctaggaacgatgagcctccaac

SEQ ID NO: 301 - DNA, 53 bp, Artificial Sequence
  Description of Artificial Sequence Synthetic primer
caagcagaagacggcatacgagatattaccgctaggaacgatgagcctccaac

SEQ ID NO: 302 - DNA, 53 bp, Artificial Sequence
  Description of Artificial Sequence Synthetic primer
caagcagaagacggcatacgagattctcctgctaggaacgatgagcctccaac

SEQ ID NO: 303 - DNA, 53 bp, Artificial Sequence
  Description of Artificial Sequence Synthetic primer
caagcagaagacggcatacgagatagatcagctaggaacgatgagcctccaac

SEQ ID NO: 304 - DNA, 53 bp, Artificial Sequence
  Description of Artificial Sequence Synthetic primer
caagcagaagacggcatacgagattcctatgctaggaacgatgagcctccaac

SEQ ID NO: 305 - DNA, 53 bp, Artificial Sequence
  Description of Artificial Sequence Synthetic primer
caagcagaagacggcatacgagatactcgggctaggaacgatgagcctccaac

SEQ ID NO: 306 - DNA, 53 bp, Artificial Sequence
  Description of Artificial Sequence Synthetic primer
caagcagaagacggcatacgagattgccatgctaggaacgatgagcctccaac

SEQ ID NO: 307 - DNA, 53 bp, Artificial Sequence
  Description of Artificial Sequence Synthetic primer
caagcagaagacggcatacgagatcatctcgctaggaacgatgagcctccaac

SEQ ID NO: 308 - DNA, 53 bp, Artificial Sequence
  Description of Artificial Sequence Synthetic primer
caagcagaagacggcatacgagatctttgagctaggaacgatgagcctccaac

SEQ ID NO: 309 - DNA, 53 bp, Artificial Sequence
  Description of Artificial Sequence Synthetic primer
caagcagaagacggcatacgagattcgcatgctaggaacgatgagcctccaac

SEQ ID NO: 310 - DNA, 53 bp, Artificial Sequence
  Description of Artificial Sequence Synthetic primer
caagcagaagacggcatacgagatcacaccgctaggaacgatgagcctccaac

SEQ ID NO: 311 - DNA, 53 bp, Artificial Sequence
  Description of Artificial Sequence Synthetic primer
caagcagaagacggcatacgagatacagaagctaggaacgatgagcctccaac

SEQ ID NO: 312 - DNA, 53 bp, Artificial Sequence
  Description of Artificial Sequence Synthetic primer
caagcagaagacggcatacgagatatgtcagctaggaacgatgagcctccaac

SEQ ID NO: 313 - DNA, 53 bp, Artificial Sequence
  Description of Artificial Sequence Synthetic primer
caagcagaagacggcatacgagatctgtccgctaggaacgatgagcctccaac

SEQ ID NO: 314 - DNA, 53 bp, Artificial Sequence
  Description of Artificial Sequence Synthetic primer
caagcagaagacggcatacgagatcgatccgctaggaacgatgagcctccaac

SEQ ID NO: 315 - DNA, 53 bp, Artificial Sequence
  Description of Artificial Sequence Synthetic primer
caagcagaagacggcatacgagattggagggctaggaacgatgagcctccaac

SEQ ID NO: 316 - DNA, 53 bp, Artificial Sequence
  Description of Artificial Sequence Synthetic primer
caagcagaagacggcatacgagatcgccaagctaggaacgatgagcctccaac

SEQ ID NO: 317 - DNA, 53 bp, Artificial Sequence
  Description of Artificial Sequence Synthetic primer
caagcagaagacggcatacgagatgaataggctaggaacgatgagcctccaac

SEQ ID NO: 318 - DNA, 53 bp, Artificial Sequence
  Description of Artificial Sequence Synthetic primer
caagcagaagacggcatacgagatcaacgggctaggaacgatgagcctccaac

SEQ ID NO: 319 - DNA, 53 bp, Artificial Sequence
  Description of Artificial Sequence Synthetic primer
caagcagaagacggcatacgagatgcggaagctaggaacgatgagcctccaac

SEQ ID NO: 320 - DNA, 53 bp, Artificial Sequence
  Description of Artificial Sequence Synthetic primer
caagcagaagacggcatacgagattctgcagctaggaacgatgagcctccaac

SEQ ID NO: 321 - DNA, 53 bp, Artificial Sequence
  Description of Artificial Sequence Synthetic primer
caagcagaagacggcatacgagatgatggcgctaggaacgatgagcctccaac

SEQ ID NO: 322 - DNA, 53 bp, Artificial Sequence
  Description of Artificial Sequence Synthetic primer
caagcagaagacggcatacgagatccgatggctaggaacgatgagcctccaac

SEQ ID NO: 323 - DNA, 53 bp, Artificial Sequence
  Description of Artificial Sequence Synthetic primer
caagcagaagacggcatacgagatgatttcgctaggaacgatgagcctccaac

SEQ ID NO: 324 - DNA, 53 bp, Artificial Sequence
  Description of Artificial Sequence Synthetic primer
caagcagaagacggcatacgagatccaaacgctaggaacgatgagcctccaac

SEQ ID NO: 325 - DNA, 53 bp, Artificial Sequence
  Description of Artificial Sequence Synthetic primer
caagcagaagacggcatacgagataggatcgctaggaacgatgagcctccaac

SEQ ID NO: 326 - DNA, 53 bp, Artificial Sequence
  Description of Artificial Sequence Synthetic primer
caagcagaagacggcatacgagatcattcagctaggaacgatgagcctccaac

SEQ ID NO: 327 - DNA, 53 bp, Artificial Sequence
  Description of Artificial Sequence Synthetic primer
caagcagaagacggcatacgagatagattggctaggaacgatgagcctccaac

SEQ ID NO: 328 - DNA, 53 bp, Artificial Sequence
  Description of Artificial Sequence Synthetic primer
caagcagaagacggcatacgagatcgaagcgctaggaacgatgagcctccaac

SEQ ID NO: 329 - DNA, 53 bp, Artificial Sequence
  Description of Artificial Sequence Synthetic primer
caagcagaagacggcatacgagatggaacggctaggaacgatgagcctccaac

SEQ ID NO: 330 - DNA, 53 bp, Artificial Sequence
  Description of Artificial Sequence Synthetic primer
caagcagaagacggcatacgagatcgaccagctaggaacgatgagcctccaac

SEQ ID NO: 331 - DNA, 53 bp, Artificial Sequence
  Description of Artificial Sequence Synthetic primer
caagcagaagacggcatacgagatagcttagctaggaacgatgagcctccaac

SEQ ID NO: 332 - DNA, 53 bp, Artificial Sequence
  Description of Artificial Sequence Synthetic primer
caagcagaagacggcatacgagatttcacggctaggaacgatgagcctccaac

SEQ ID NO: 333 - DNA, 53 bp, Artificial Sequence
  Description of Artificial Sequence Synthetic primer
caagcagaagacggcatacgagatcattaggctaggaacgatgagcctccaac

SEQ ID NO: 334 - DNA, 53 bp, Artificial Sequence
  Description of Artificial Sequence Synthetic primer
caagcagaagacggcatacgagattaggaggctaggaacgatgagcctccaac

SEQ ID NO: 335 - DNA, 53 bp, Artificial Sequence
  Description of Artificial Sequence Synthetic primer
caagcagaagacggcatacgagatctaccggctaggaacgatgagcctccaac

SEQ ID NO: 336 - DNA, 53 bp, Artificial Sequence
  Description of Artificial Sequence Synthetic primer
caagcagaagacggcatacgagatatatccgctaggaacgatgagcctccaac

SEQ ID NO: 337 - DNA, 53 bp, Artificial Sequence
  Description of Artificial Sequence Synthetic primer
caagcagaagacggcatacgagataatggagctaggaacgatgagcctccaac

SEQ ID NO: 338 - DNA, 53 bp, Artificial Sequence
  Description of Artificial Sequence Synthetic primer
caagcagaagacggcatacgagatttcgacgctaggaacgatgagcctccaac

SEQ ID NO: 339 - DNA, 53 bp, Artificial Sequence
  Description of Artificial Sequence Synthetic primer
caagcagaagacggcatacgagataaccgggctaggaacgatgagcctccaac

SEQ ID NO: 340 - DNA, 53 bp, Artificial Sequence
  Description of Artificial Sequence Synthetic primer
caagcagaagacggcatacgagataaggccgctaggaacgatgagcctccaac

SEQ ID NO: 341 - DNA, 53 bp, Artificial Sequence
  Description of Artificial Sequence Synthetic primer
caagcagaagacggcatacgagatcgtcctgctaggaacgatgagcctccaac

SEQ ID NO: 342 - DNA, 53 bp, Artificial Sequence
  Description of Artificial Sequence Synthetic primer
caagcagaagacggcatacgagataagagcgctaggaacgatgagcctccaac

SEQ ID NO: 343 - DNA, 53 bp, Artificial Sequence
  Description of Artificial Sequence Synthetic primer
caagcagaagacggcatacgagatgtgaaagctaggaacgatgagcctccaac

SEQ ID NO: 344 - DNA, 53 bp, Artificial Sequence
  Description of Artificial Sequence Synthetic primer
caagcagaagacggcatacgagatacgaacgctaggaacgatgagcctccaac

SEQ ID NO: 345 - DNA, 53 bp, Artificial Sequence
  Description of Artificial Sequence Synthetic primer
caagcagaagacggcatacgagatccgaaagctaggaacgatgagcctccaac

SEQ ID NO: 346 - DNA, 53 bp, Artificial Sequence
  Description of Artificial Sequence Synthetic primer
caagcagaagacggcatacgagatcaaggcgctaggaacgatgagcctccaac

SEQ ID NO: 347 - DNA, 53 bp, Artificial Sequence
  Description of Artificial Sequence Synthetic primer
caagcagaagacggcatacgagatatgtgcgctaggaacgatgagcctccaac

SEQ ID NO: 348 - DNA, 53 bp, Artificial Sequence
  Description of Artificial Sequence Synthetic primer
caagcagaagacggcatacgagatcccttggctaggaacgatgagcctccaac

SEQ ID NO: 349 - DNA, 53 bp, Artificial Sequence
  Description of Artificial Sequence Synthetic primer
caagcagaagacggcatacgagatgagaaggctaggaacgatgagcctccaac

SEQ ID NO: 350 - DNA, 53 bp, Artificial Sequence
  Description of Artificial Sequence Synthetic primer
caagcagaagacggcatacgagatgtagttgctaggaacgatgagcctccaac

SEQ ID NO: 351 - DNA, 53 bp, Artificial Sequence
  Description of Artificial Sequence Synthetic primer
caagcagaagacggcatacgagattggtgcgctaggaacgatgagcctccaac

SEQ ID NO: 352 - DNA, 53 bp, Artificial Sequence
  Description of Artificial Sequence Synthetic primer
caagcagaagacggcatacgagatgactatgctaggaacgatgagcctccaac

SEQ ID NO: 353 - DNA, 53 bp, Artificial Sequence
  Description of Artificial Sequence Synthetic primer
caagcagaagacggcatacgagatctcgcagctaggaacgatgagcctccaac

SEQ ID NO: 354 - DNA, 53 bp, Artificial Sequence
  Description of Artificial Sequence Synthetic primer
caagcagaagacggcatacgagattcttgtgctaggaacgatgagcctccaac

SEQ ID NO: 355 - DNA, 53 bp, Artificial Sequence
  Description of Artificial Sequence Synthetic primer
caagcagaagacggcatacgagatgcacaagctaggaacgatgagcctccaac

SEQ ID NO: 356 - DNA, 53 bp, Artificial Sequence
  Description of Artificial Sequence Synthetic primer
caagcagaagacggcatacgagatcctttagctaggaacgatgagcctccaac

SEQ ID NO: 357 - DNA, 53 bp, Artificial Sequence
  Description of Artificial Sequence Synthetic primer
caagcagaagacggcatacgagatacgttggctaggaacgatgagcctccaac

SEQ ID NO: 358 - DNA, 53 bp, Artificial Sequence
  Description of Artificial Sequence Synthetic primer
caagcagaagacggcatacgagataagcgtgctaggaacgatgagcctccaac

SEQ ID NO: 359 - DNA, 53 bp, Artificial Sequence
  Description of Artificial Sequence Synthetic primer
caagcagaagacggcatacgagataggcaagctaggaacgatgagcctccaac

SEQ ID NO: 360 - DNA, 53 bp, Artificial Sequence
  Description of Artificial Sequence Synthetic primer
caagcagaagacggcatacgagattgagccgctaggaacgatgagcctccaac

SEQ ID NO: 361 - DNA, 53 bp, Artificial Sequence
  Description of Artificial Sequence Synthetic primer
caagcagaagacggcatacgagattgagaagctaggaacgatgagcctccaac

SEQ ID NO: 362 - DNA, 53 bp, Artificial Sequence
  Description of Artificial Sequence Synthetic primer
caagcagaagacggcatacgagatgtacaggctaggaacgatgagcctccaac

SEQ ID NO: 363 - DNA, 53 bp, Artificial Sequence
  Description of Artificial Sequence Synthetic primer
caagcagaagacggcatacgagatacggaggctaggaacgatgagcctccaac

SEQ ID NO: 364 - DNA, 53 bp, Artificial Sequence
  Description of Artificial Sequence Synthetic primer
caagcagaagacggcatacgagatacatacgctaggaacgatgagcctccaac

SEQ ID NO: 365 - DNA, 53 bp, Artificial Sequence
  Description of Artificial Sequence Synthetic primer
caagcagaagacggcatacgagatcagccagctaggaacgatgagcctccaac

SEQ ID NO: 366 - DNA, 53 bp, Artificial Sequence
  Description of Artificial Sequence Synthetic primer
caagcagaagacggcatacgagattgtcaagctaggaacgatgagcctccaac

SEQ ID NO: 367 - DNA, 53 bp, Artificial Sequence
  Description of Artificial Sequence Synthetic primer
caagcagaagacggcatacgagattattgggctaggaacgatgagcctccaac

SEQ ID NO: 368 - DNA, 53 bp, Artificial Sequence
  Description of Artificial Sequence Synthetic primer
caagcagaagacggcatacgagatagaccggctaggaacgatgagcctccaac

SEQ ID NO: 369 - DNA, 53 bp, Artificial Sequence
  Description of Artificial Sequence Synthetic primer
caagcagaagacggcatacgagatgccaacgctaggaacgatgagcctccaac

SEQ ID NO: 370 - DNA, 53 bp, Artificial Sequence
  Description of Artificial Sequence Synthetic primer
caagcagaagacggcatacgagatatgtaggctaggaacgatgagcctccaac

SEQ ID NO: 371 - DNA, 53 bp, Artificial Sequence
  Description of Artificial Sequence Synthetic primer
caagcagaagacggcatacgagatcgctacgctaggaacgatgagcctccaac

SEQ ID NO: 372 - DNA, 53 bp, Artificial Sequence
  Description of Artificial Sequence Synthetic primer
caagcagaagacggcatacgagatcactcggctaggaacgatgagcctccaac

SEQ ID NO: 373 - DNA, 53 bp, Artificial Sequence
  Description of Artificial Sequence Synthetic primer
caagcagaagacggcatacgagatgctattgctaggaacgatgagcctccaac

SEQ ID NO: 374 - DNA, 53 bp, Artificial Sequence
  Description of Artificial Sequence Synthetic primer
caagcagaagacggcatacgagatctccacgctaggaacgatgagcctccaac

SEQ ID NO: 375 - DNA, 53 bp, Artificial Sequence
  Description of Artificial Sequence Synthetic primer
caagcagaagacggcatacgagatgttagcgctaggaacgatgagcctccaac

SEQ ID NO: 376 - DNA, 53 bp, Artificial Sequence
  Description of Artificial Sequence Synthetic primer
caagcagaagacggcatacgagataaacctgctaggaacgatgagcctccaac

SEQ ID NO: 377 - DNA, 53 bp, Artificial Sequence
  Description of Artificial Sequence Synthetic primer
caagcagaagacggcatacgagatctagatgctaggaacgatgagcctccaac

SEQ ID NO: 378 - DNA, 53 bp, Artificial Sequence
  Description of Artificial Sequence Synthetic primer
caagcagaagacggcatacgagataccgtcgctaggaacgatgagcctccaac

SEQ ID NO: 379 - DNA, 53 bp, Artificial Sequence
  Description of Artificial Sequence Synthetic primer
caagcagaagacggcatacgagattcacccgctaggaacgatgagcctccaac

SEQ ID NO: 380 - DNA, 53 bp, Artificial Sequence
  Description of Artificial Sequence Synthetic primer
caagcagaagacggcatacgagatgaagatgctaggaacgatgagcctccaac

SEQ ID NO: 381 - DNA, 53 bp, Artificial Sequence
  Description of Artificial Sequence Synthetic primer
caagcagaagacggcatacgagattggctcgctaggaacgatgagcctccaac

SEQ ID NO: 382 - DNA, 53 bp, Artificial Sequence
  Description of Artificial Sequence Synthetic primer
caagcagaagacggcatacgagatggttatgctaggaacgatgagcctccaac

SEQ ID NO: 383 - DNA, 53 bp, Artificial Sequence
  Description of Artificial Sequence Synthetic primer
caagcagaagacggcatacgagatacacttgctaggaacgatgagcctccaac

SEQ ID NO: 384 - DNA, 53 bp, Artificial Sequence
  Description of Artificial Sequence Synthetic primer
caagcagaagacggcatacgagatggaagagctaggaacgatgagcctccaac

SEQ ID NO: 385 - DNA, 53 bp, Artificial Sequence
  Description of Artificial Sequence Synthetic primer
caagcagaagacggcatacgagattacgtggctaggaacgatgagcctccaac

SEQ ID NO: 386 - DNA, 53 bp, Artificial Sequence
  Description of Artificial Sequence Synthetic primer
caagcagaagacggcatacgagatcttgacgctaggaacgatgagcctccaac

SEQ ID NO: 387 - DNA, 53 bp, Artificial Sequence
  Description of Artificial Sequence Synthetic primer
caagcagaagacggcatacgagatatgcccgctaggaacgatgagcctccaac

SEQ ID NO: 388 - DNA, 53 bp, Artificial Sequence
  Description of Artificial Sequence Synthetic primer
caagcagaagacggcatacgagatattcacgctaggaacgatgagcctccaac

SEQ ID NO: 389 - DNA, 53 bp, Artificial Sequence
  Description of Artificial Sequence Synthetic primer
caagcagaagacggcatacgagatgcttacgctaggaacgatgagcctccaac

SEQ ID NO: 390 - DNA, 53 bp, Artificial Sequence
  Description of Artificial Sequence Synthetic primer
caagcagaagacggcatacgagatttctgcgctaggaacgatgagcctccaac

SEQ ID NO: 391 - DNA, 53 bp, Artificial Sequence
  Description of Artificial Sequence Synthetic primer
caagcagaagacggcatacgagattgtatggctaggaacgatgagcctccaac

SEQ ID NO: 392 - DNA, 53 bp, Artificial Sequence
  Description of Artificial Sequence Synthetic primer
caagcagaagacggcatacgagattgacgcgctaggaacgatgagcctccaac

SEQ ID NO: 393 - DNA, 53 bp, Artificial Sequence
  Description of Artificial Sequence Synthetic primer
caagcagaagacggcatacgagatgtgttagctaggaacgatgagcctccaac

SEQ ID NO: 394 - DNA, 53 bp, Artificial Sequence
  Description of Artificial Sequence Synthetic primer
caagcagaagacggcatacgagatcatcgagctaggaacgatgagcctccaac

SEQ ID NO: 395 - DNA, 53 bp, Artificial Sequence
  Description of Artificial Sequence Synthetic primer
caagcagaagacggcatacgagattgaggtgctaggaacgatgagcctccaac

SEQ ID NO: 396 - DNA, 53 bp, Artificial Sequence
  Description of Artificial Sequence Synthetic primer
caagcagaagacggcatacgagatggtccagctaggaacgatgagcctccaac

SEQ ID NO: 397 - DNA, 53 bp, Artificial Sequence
  Description of Artificial Sequence Synthetic primer
caagcagaagacggcatacgagattatgctgctaggaacgatgagcctccaac

SEQ ID NO: 398 - DNA, 53 bp, Artificial Sequence
  Description of Artificial Sequence Synthetic primer
caagcagaagacggcatacgagatttcatcgctaggaacgatgagcctccaac

SEQ ID NO: 399 - DNA, 53 bp, Artificial Sequence
  Description of Artificial Sequence Synthetic primer
caagcagaagacggcatacgagatcctctggctaggaacgatgagcctccaac

SEQ ID NO: 400 - DNA, 53 bp, Artificial Sequence
  Description of Artificial Sequence Synthetic primer
caagcagaagacggcatacgagatccaagggctaggaacgatgagcctccaac

SEQ ID NO: 401 - DNA, 53 bp, Artificial Sequence
  Description of Artificial Sequence Synthetic primer
caagcagaagacggcatacgagattaatgcgctaggaacgatgagcctccaac

SEQ ID NO: 402 - DNA, 53 bp, Artificial Sequence
  Description of Artificial Sequence Synthetic primer
caagcagaagacggcatacgagatagggcagctaggaacgatgagcctccaac

SEQ ID NO: 403 - DNA, 53 bp, Artificial Sequence
  Description of Artificial Sequence Synthetic primer
caagcagaagacggcatacgagattagagagctaggaacgatgagcctccaac

SEQ ID NO: 404 - DNA, 53 bp, Artificial Sequence
  Description of Artificial Sequence Synthetic primer
caagcagaagacggcatacgagatagcacagctaggaacgatgagcctccaac

SEQ ID NO: 405 - DNA, 53 bp, Artificial Sequence
  Description of Artificial Sequence Synthetic primer
caagcagaagacggcatacgagatagagtcgctaggaacgatgagcctccaac

SEQ ID NO: 406 - DNA, 53 bp, Artificial Sequence
  Description of Artificial Sequence Synthetic primer
caagcagaagacggcatacgagatactcaagctaggaacgatgagcctccaac

SEQ ID NO: 407 - DNA, 53 bp, Artificial Sequence
  Description of Artificial Sequence Synthetic primer
caagcagaagacggcatacgagatgtgacggctaggaacgatgagcctccaac

SEQ ID NO: 408 - DNA, 53 bp, Artificial Sequence
  Description of Artificial Sequence Synthetic primer
caagcagaagacggcatacgagatggtagggctaggaacgatgagcctccaac

SEQ ID NO: 409 - DNA, 53 bp, Artificial Sequence
  Description of Artificial Sequence Synthetic primer
caagcagaagacggcatacgagatctgaatgctaggaacgatgagcctccaac

SEQ ID NO: 410 - DNA, 53 bp, Artificial Sequence
  Description of Artificial Sequence Synthetic primer
caagcagaagacggcatacgagatgtctacgctaggaacgatgagcctccaac

SEQ ID NO: 411 - DNA, 53 bp, Artificial Sequence
  Description of Artificial Sequence Synthetic primer
caagcagaagacggcatacgagattcggacgctaggaacgatgagcctccaac

SEQ ID NO: 412 - DNA, 53 bp, Artificial Sequence
  Description of Artificial Sequence Synthetic primer
caagcagaagacggcatacgagataactacgctaggaacgatgagcctccaac

SEQ ID NO: 413 - DNA, 53 bp, Artificial Sequence
  Description of Artificial Sequence Synthetic primer
caagcagaagacggcatacgagataaggttgctaggaacgatgagcctccaac

SEQ ID NO: 414 - DNA, 53 bp, Artificial Sequence
  Description of Artificial Sequence Synthetic primer
caagcagaagacggcatacgagatagggacgctaggaacgatgagcctccaac

SEQ ID NO: 415 - DNA, 53 bp, Artificial Sequence
  Description of Artificial Sequence Synthetic primer
caagcagaagacggcatacgagatacgcgagctaggaacgatgagcctccaac

SEQ ID NO: 416 - DNA, 53 bp, Artificial Sequence
  Description of Artificial Sequence Synthetic primer
caagcagaagacggcatacgagatttgctagctaggaacgatgagcctccaac

SEQ ID NO: 417 - DNA, 53 bp, Artificial Sequence
  Description of Artificial Sequence Synthetic primer
caagcagaagacggcatacgagatataacggctaggaacgatgagcctccaac

SEQ ID NO: 418 - DNA, 53 bp, Artificial Sequence
  Description of Artificial Sequence Synthetic primer
caagcagaagacggcatacgagatccggtagctaggaacgatgagcctccaac

SEQ ID NO: 419 - DNA, 53 bp, Artificial Sequence
  Description of Artificial Sequence Synthetic primer
caagcagaagacggcatacgagatagctaggctaggaacgatgagcctccaac

SEQ ID NO: 420 - DNA, 44 bp, Artificial Sequence
  Description of Artificial Sequence Synthetic oligonucleotide
ggctcatcgttcctattcagctgcagatgttatcgaggtccgac

SEQ ID NO: 421 - DNA, 18 bp, Artificial Sequence
  Description of Artificial Sequence Synthetic oligonucleotide
gtcatatcggtcactgtt

SEQ ID NO: 422 - DNA, 26 bp, Artificial Sequence
  Description of Artificial Sequence Synthetic oligonucleotide
gttggaggctcatcgttcctattcag

SEQ ID NO: 423 - DNA, 22 bp, Artificial Sequence
  Description of Artificial Sequence Synthetic oligonucleotide
gcagatgttatcgaggtccgac

SEQ ID NO: 424 - DNA, 20 bp, Artificial Sequence
  Description of Artificial Sequence Synthetic oligonucleotide
gatcaggatacacactaccc

SEQ ID NO: 425 - DNA, 18 bp, Artificial Sequence
  Description of Artificial Sequence Synthetic primer
gtcatatcggtcactgtt

SEQ ID NO: 426 - DNA, 48 bp, Artificial Sequence
  Description of Artificial Sequence Synthetic oligonucleotide
gttggaggctcatcgttcctattcaggcagatgttatcgaggtccgac

SEQ ID NO: 427 - DNA, 20 bp, Artificial Sequence
  Description of Artificial Sequence Synthetic primer
gatcaggatacacactaccc

SEQ ID NO: 428 - DNA, 15 bp, Artificial Sequence
  Description of Artificial Sequence Synthetic primer
aggaccggatcaact

SEQ ID NO: 429 - DNA, 30 bp, Artificial Sequence
  Description of Artificial Sequence Synthetic oligonucleotide
cttcagcttcccgatatccgacggtagtgt

SEQ ID NO: 430 - DNA, 15 bp, Artificial Sequence
  Description of Artificial Sequence Synthetic primer
cattgcgtgaaccga